SEQUENCE LISTING (237 sequences)

SEQ ID NO: 1 - protein, 15 aa, Artificial Sequence
  Description of Artificial Sequence Synthetic peptide
YKRWIILGLNKIVRM

SEQ ID NO: 2 - protein, 16 aa, Artificial Sequence
  Description of Artificial Sequence Synthetic peptide
KQIINMWQVVGKAMYA

SEQ ID NO: 3 - protein, 39 aa, Artificial Sequence
  Description of Artificial Sequence Synthetic peptide
KQIINMWQEVGKAMYATRPNNNTRRRLSIGPGRAFYARR

SEQ ID NO: 4 - protein, 39 aa, Artificial Sequence
  Description of Artificial Sequence Synthetic peptide
KQIINMWQVVGKAMYATRPNNNTRRRLSIGPGRAFYARR

SEQ ID NO: 5 - protein, 39 aa, Artificial Sequence
  Description of Artificial Sequence Synthetic peptide
KQIINMWQEVGKAMYATRPNNNTRERLSIGPGRAFYARR

SEQ ID NO: 6 - protein, 39 aa, Artificial Sequence
  Description of Artificial Sequence Synthetic peptide
KQIINMWQVVGKAMYATRPNNNTRERLSIGPGRAFYARR

SEQ ID NO: 7 - protein, 17 aa, Artificial Sequence
  Description of Artificial Sequence Synthetic peptide
YKRWIILGLNKIVRMYS

SEQ ID NO: 8 - protein, 39 aa, Artificial Sequence
  Description of Artificial Sequence Synthetic peptide
KQIINMWQVVGKAMYATRPNNNTRKSIRIGPGQTFYATG

SEQ ID NO: 9 - protein, 39 aa, Artificial Sequence
  Description of Artificial Sequence Synthetic peptide
KQIINMWQVVGKAMYATRPNNNTRKSIRIGPGQTFYARG

SEQ ID NO: 10 - protein, 39 aa, Artificial Sequence
  Description of Artificial Sequence Synthetic peptide
KQIINMWQVVGKAMYATRPNNNTRKSIRIGPGQTFYAAG

SEQ ID NO: 11 - protein, 39 aa, Artificial Sequence
  Description of Artificial Sequence Synthetic peptide
KQIINMWQVVGKAMYAIRPNNNTRKSVRIGPGQTFYATG

SEQ ID NO: 12 - protein, 39 aa, Artificial Sequence
  Description of Artificial Sequence Synthetic peptide
KQIINMWQVVGKAMYATRPNNNTRKSIRIGPGQTFFATG

SEQ ID NO: 13 - protein, 39 aa, Artificial Sequence
  Description of Artificial Sequence Synthetic peptide
KQIINMWQVVGKAMYATRPNNNTRKSIRIGPGQTFYATN

SEQ ID NO: 14 - protein, 39 aa, Artificial Sequence
  Description of Artificial Sequence Synthetic peptide
KQIINMWQVVGKAMYATRPNNNTRESIRIGPGQTFYATG

SEQ ID NO: 15 - protein, 39 aa, Artificial Sequence
  Description of Artificial Sequence Synthetic peptide
KQIINMWQVVGKAMYATRPNNNTRRSIRIGPGQAFYATG

SEQ ID NO: 16 - protein, 39 aa, Artificial Sequence
  Description of Artificial Sequence Synthetic peptide
KQIINMWQVVGKAMYATRPNNNTRKGIRIGPGQTFYATG

SEQ ID NO: 17 - protein, 39 aa, Artificial Sequence
  Description of Artificial Sequence Synthetic peptide
KQIINMWQVVGKAMYATRPSNNTRKSIRIGPGQAFYATG

SEQ ID NO: 18 - protein, 39 aa, Artificial Sequence
  Description of Artificial Sequence Synthetic peptide
KQIINMWQVVGKAMYATRPSNNTRKSIRIGPGQTFYATN

SEQ ID NO: 19 - protein, 39 aa, Artificial Sequence
  Description of Artificial Sequence Synthetic peptide
KQIINMWQVVGKAMYATRPSNNTRESIRIGPGQTFYATG

SEQ ID NO: 20 - protein, 39 aa, Artificial Sequence
  Description of Artificial Sequence Synthetic peptide
KQIINMWQVVGKAMYATRPNNNTRKSMRIGPGQTFYATG

SEQ ID NO: 21 - protein, 39 aa, Artificial Sequence
  Description of Artificial Sequence Synthetic peptide
KQIINMWQVVGKAMYATRPGNNTRKSMRIGPGQTFYATG

SEQ ID NO: 22 - protein, 39 aa, Artificial Sequence
  Description of Artificial Sequence Synthetic peptide
KQIINMWQVVGKAMYATRPGNNTRKSIRIGPGQTLYATG

SEQ ID NO: 23 - protein, 39 aa, Artificial Sequence
  Description of Artificial Sequence Synthetic peptide
KQIINMWQVVGKAMYAVRPNNNTRKSVRIGPGQTSYATG

SEQ ID NO: 24 - protein, 39 aa, Artificial Sequence
  Description of Artificial Sequence Synthetic peptide
KQIINMWQVVGKAMYATRPGNNTRRSIRIGPGQTFYATG

SEQ ID NO: 25 - protein, 39 aa, Artificial Sequence
  Description of Artificial Sequence Synthetic peptide
KQIINMWQVVGKAMYAIRPGNNTRKSVRIGPGQTFYATG

SEQ ID NO: 26 - protein, 39 aa, Artificial Sequence
  Description of Artificial Sequence Synthetic peptide
KQIINMWQVVGKAMYATRPNNNTRKSIRIGPGQAFYATN

SEQ ID NO: 27 - protein, 39 aa, Artificial Sequence
  Description of Artificial Sequence Synthetic peptide
KQIINMWQVVGKAMYATRPNNNTRQSIRIGPGQAFYATK

SEQ ID NO: 28 - protein, 39 aa, Artificial Sequence
  Description of Artificial Sequence Synthetic peptide
KQIINMWQVVGKAMYATRPGNNTRKSIRIGPGQAFFATG

SEQ ID NO: 29 - protein, 39 aa, Artificial Sequence
  Description of Artificial Sequence Synthetic peptide
KQIINMWQVVGKAMYATRPGNNTRKSVRIGPGQAFYATN

SEQ ID NO: 30 - protein, 39 aa, Artificial Sequence
  Description of Artificial Sequence Synthetic peptide
KQIINMWQVVGKAMYATRPNNNTRKGIHIGPGQAFYAAG

SEQ ID NO: 31 - protein, 39 aa, Artificial Sequence
  Description of Artificial Sequence Synthetic peptide
KQIINMWQVVGKAMYATRPNNNTRKGIGIGPGQTFFATE

SEQ ID NO: 32 - protein, 39 aa, Artificial Sequence
  Description of Artificial Sequence Synthetic peptide
KQIINMWQVVGKAMYATRPGNNTRESIGIGPGQAFYATG

SEQ ID NO: 33 - protein, 37 aa, Artificial Sequence
  Description of Artificial Sequence Synthetic peptide
KQIINMWQVVGKAMYARPNNNTRRNIHIGLGRRFYAT

SEQ ID NO: 34 - protein, 38 aa, Artificial Sequence
  Description of Artificial Sequence Synthetic peptide
KQIINMWQVVGKAMYARPNNNTRRSVRIGPGGAMFRTG

SEQ ID NO: 35 - protein, 38 aa, Artificial Sequence
  Description of Artificial Sequence Synthetic peptide
KQIINMWQVVGKAMYARPNNNTRRSIPIGPGRAFYTTG

SEQ ID NO: 36 - protein, 37 aa, Artificial Sequence
  Description of Artificial Sequence Synthetic peptide
KQIINMWQVVGKAMYARPDNNTVRKIPIGPGSSFYTT

SEQ ID NO: 37 - protein, 38 aa, Artificial Sequence
  Description of Artificial Sequence Synthetic peptide
KQIINMWQVVGKAMYARPIKIERKRIPLGLGKAFYTTK

SEQ ID NO: 38 - protein, 38 aa, Artificial Sequence
  Description of Artificial Sequence Synthetic peptide
KQIINMWQVVGKAMYARPSNNTRKGIHLGPGRAIYATE

SEQ ID NO: 39 - protein, 38 aa, Artificial Sequence
  Description of Artificial Sequence Synthetic peptide
KQIINMWQVVGKAMYARPSNNTRKGIHMGPGKAIYTTD

SEQ ID NO: 40 - protein, 38 aa, Artificial Sequence
  Description of Artificial Sequence Synthetic peptide
KQIINMWQVVGKAMYARPGNTTRRGIPIGPGRAFFTTG

SEQ ID NO: 41 - protein, 38 aa, Artificial Sequence
  Description of Artificial Sequence Synthetic peptide
KQIINMWQVVGKAMYARPNNNTRKSIRIGPGRAVYATD

SEQ ID NO: 42 - protein, 38 aa, Artificial Sequence
  Description of Artificial Sequence Synthetic peptide
KQIINMWQVVGKAMYARPGNNTRRRISIGPGRAFVATK

SEQ ID NO: 43 - protein, 38 aa, Artificial Sequence
  Description of Artificial Sequence Synthetic peptide
KQIINMWQVVGKAMYARPNNNTRRSIHLGMGRALYATG

SEQ ID NO: 44 - protein, 38 aa, Artificial Sequence
  Description of Artificial Sequence Synthetic peptide
KQIINMWQVVGKAMYARPSNNTRRSIHMGLGRAFYTTG

SEQ ID NO: 45 - protein, 38 aa, Artificial Sequence
  Description of Artificial Sequence Synthetic peptide
KQIINMWQVVGKAMYARPNNNTRKGINIGPGRAFYATG

SEQ ID NO: 46 - protein, 38 aa, Artificial Sequence
  Description of Artificial Sequence Synthetic peptide
KQIINMWQVVGKAMYARPNNNTRKGIHIGPGRTFFATG

SEQ ID NO: 47 - protein, 41 aa, Artificial Sequence
  Description of Artificial Sequence Synthetic peptide
KQIINMWQVVGKAMYARPNNNTRKRIRIGHIGPGRAFYATG

SEQ ID NO: 48 - protein, 37 aa, Artificial Sequence
  Description of Artificial Sequence Synthetic peptide
KQIINMWQVVGKAMYARPSINKRRHIHIGPGRAFYAT

SEQ ID NO: 49 - protein, 38 aa, Artificial Sequence
  Description of Artificial Sequence Synthetic peptide
KQIINMWQVVGKAMYARLYNYRRKGIHIGPGRAIYATG

SEQ ID NO: 50 - protein, 38 aa, Artificial Sequence
  Description of Artificial Sequence Synthetic peptide
KQIINMWQVVGKAMYARPNRHTGKSIRMGLGRAWHTTR

SEQ ID NO: 51 - protein, 38 aa, Artificial Sequence
  Description of Artificial Sequence Synthetic peptide
KQIINMWQVVGKAMYARPNNNTRKSINIGPGRAFYTTG

SEQ ID NO: 52 - protein, 38 aa, Artificial Sequence
  Description of Artificial Sequence Synthetic peptide
KQIINMWQVVGKAMYARPNNNTRKSIQIGPGRAFYTTG

SEQ ID NO: 53 - protein, 38 aa, Artificial Sequence
  Description of Artificial Sequence Synthetic peptide
KQIINMWQVVGKAMYARPNNNTRKSIHIGPGRAFYTTG

SEQ ID NO: 54 - protein, 38 aa, Artificial Sequence
  Description of Artificial Sequence Synthetic peptide
KQIINMWQVVGKAMYARPNNNTRKSIHIAPGRAFYTTG

SEQ ID NO: 55 - protein, 38 aa, Artificial Sequence
  Description of Artificial Sequence Synthetic peptide
KQIINMWQVVGKAMYARPNNNTRKSIHIGPGRAFYATE

SEQ ID NO: 56 - protein, 38 aa, Artificial Sequence
  Description of Artificial Sequence Synthetic peptide
KQIINMWQVVGKAMYARPNNNTRRRISMGPGRVLYTTG

SEQ ID NO: 57 - protein, 38 aa, Artificial Sequence
  Description of Artificial Sequence Synthetic peptide
KQIINMWQVVGKAMYARPNNNTRKRISLGPGRVYYTTG

SEQ ID NO: 58 - protein, 38 aa, Artificial Sequence
  Description of Artificial Sequence Synthetic peptide
KQIINMWQVVGKAMYARPNNNTRKRMTLGPGKVFYTTG

SEQ ID NO: 59 - protein, 38 aa, Artificial Sequence
  Description of Artificial Sequence Synthetic peptide
KQIINMWQVVGKAMYARPDNTIKQRIIHIGPGRPFYTT

SEQ ID NO: 60 - protein, 40 aa, Artificial Sequence
  Description of Artificial Sequence Synthetic peptide
KQIINMWQVVGKAMYARPNYNETKRIRIHRGYGRSFVTVR

SEQ ID NO: 61 - protein, 38 aa, Artificial Sequence
  Description of Artificial Sequence Synthetic peptide
KQIINMWQVVGKAMYARPGNNTRGSIHLHPGRKFYYSR

SEQ ID NO: 62 - protein, 38 aa, Artificial Sequence
  Description of Artificial Sequence Synthetic peptide
KQIINMWQVVGKAMYARPNNNTRKSINMGPGRAFYTTG

SEQ ID NO: 63 - protein, 24 aa, Artificial Sequence
  Description of Artificial Sequence Synthetic peptide
TRPNNNTRKSIHIGPGRAFYATED

SEQ ID NO: 64 - protein, 25 aa, Artificial Sequence
  Description of Artificial Sequence Synthetic peptide
TRPNNNTRKSIHIGPGRAFYATEDE

SEQ ID NO: 65 - protein, 25 aa, Artificial Sequence
  Description of Artificial Sequence Synthetic peptide
TRPNNNRRKSIHIGPGRAFYATEDE

SEQ ID NO: 66 - protein, 24 aa, Artificial Sequence
  Description of Artificial Sequence Synthetic peptide
TRPNNNTRKSIHIGPGRAFYATED

SEQ ID NO: 67 - protein, 25 aa, Artificial Sequence
  Description of Artificial Sequence Synthetic peptide
TRPNNNTRKSIHIGPGRAFYATEDE

SEQ ID NO: 68 - protein, 25 aa, Artificial Sequence
  Description of Artificial Sequence Synthetic peptide
TRPNNNRRKSIHIGPGRAFYATEDE

SEQ ID NO: 69 - protein, 25 aa, Artificial Sequence
  Description of Artificial Sequence Synthetic peptide
TRPNNNRRKSIHIGPGRAFYATEEE

SEQ ID NO: 70 - protein, 25 aa, Artificial Sequence
  Description of Artificial Sequence Synthetic peptide
TRPNNNRRKSIHIGPGRAFYATEEE

SEQ ID NO: 71 - protein, 23 aa, Artificial Sequence
  Description of Artificial Sequence Synthetic peptide
RPNNNTRKSIHIGPGRAFYTTGD

SEQ ID NO: 72 - protein, 24 aa, Artificial Sequence
  Description of Artificial Sequence Synthetic peptide
RPNNNTRKSIHIGPGRAFYTTGDE

SEQ ID NO: 73 - protein, 24 aa, Artificial Sequence
  Description of Artificial Sequence Synthetic peptide
RPNNNRRKSIHIGPGRAFYTTGDE

SEQ ID NO: 74 - protein, 24 aa, Artificial Sequence
  Description of Artificial Sequence Synthetic peptide
RPNNNRRKSIHIGPGRAFYTTGEE

SEQ ID NO: 75 - protein, 23 aa, Artificial Sequence
  Description of Artificial Sequence Synthetic peptide
RPNNNTRKSIHIGPGRAFYTTGD

SEQ ID NO: 76 - protein, 24 aa, Artificial Sequence
  Description of Artificial Sequence Synthetic peptide
RPNNNTRKSIHIGPGRAFYTTGDE

SEQ ID NO: 77 - protein, 24 aa, Artificial Sequence
  Description of Artificial Sequence Synthetic peptide
RPNNNRRKSIHIGPGRAFYTTGDE

SEQ ID NO: 78 - protein, 24 aa, Artificial Sequence
  Description of Artificial Sequence Synthetic peptide
RPNNNRRKSIHIGPGRAFYTTGEE

SEQ ID NO: 79 - protein, 23 aa, Artificial Sequence
  Description of Artificial Sequence Synthetic peptide
RPNNNTRKSINIGPGRAFYTTGD

SEQ ID NO: 80 - protein, 24 aa, Artificial Sequence
  Description of Artificial Sequence Synthetic peptide
RPNNNTRKSINIGPGRAFYTTGDE

SEQ ID NO: 81 - protein, 24 aa, Artificial Sequence
  Description of Artificial Sequence Synthetic peptide
RPNNRTRKSINIGPGRAFYTTGDE

SEQ ID NO: 82 - protein, 24 aa, Artificial Sequence
  Description of Artificial Sequence Synthetic peptide
RPNNRTRKSINIGPGRAFYTTGEE

SEQ ID NO: 83 - protein, 23 aa, Artificial Sequence
  Description of Artificial Sequence Synthetic peptide
RPNNNTRKSINIGPGRAFYTTGD

SEQ ID NO: 84 - protein, 24 aa, Artificial Sequence
  Description of Artificial Sequence Synthetic peptide
RPNNNTRKSINIGPGRAFYTTGDE

SEQ ID NO: 85 - protein, 24 aa, Artificial Sequence
  Description of Artificial Sequence Synthetic peptide
RPNNRTRKSINIGPGRAFYTTGDE

SEQ ID NO: 86 - protein, 24 aa, Artificial Sequence
  Description of Artificial Sequence Synthetic peptide
RPNNRTRKSINIGPGRAFYTTGEE

SEQ ID NO: 87 - protein, 22 aa, Artificial Sequence
  Description of Artificial Sequence Synthetic peptide
RPSNNTRKGIHMGPGKAIYTTD

SEQ ID NO: 88 - protein, 23 aa, Artificial Sequence
  Description of Artificial Sequence Synthetic peptide
RPSNNTRKGIHMGPGKAIYTTDD

SEQ ID NO: 89 - protein, 24 aa, Artificial Sequence
  Description of Artificial Sequence Synthetic peptide
RPSNNTRKGIHMGPGKAIYTTDDE

SEQ ID NO: 90 - protein, 23 aa, Artificial Sequence
  Description of Artificial Sequence Synthetic peptide
RPSNNRRKGIHMGPGKAIYTTDE

SEQ ID NO: 91 - protein, 25 aa, Artificial Sequence
  Description of Artificial Sequence Synthetic peptide
RPSNNNRRKGIHMGPGKAIYTTDEE

SEQ ID NO: 92 - protein, 23 aa, Artificial Sequence
  Description of Artificial Sequence Synthetic peptide
RPSNNTRKGIHMGPGKAIYTTDD

SEQ ID NO: 93 - protein, 24 aa, Artificial Sequence
  Description of Artificial Sequence Synthetic peptide
RPSNNTRKGIHMGPGKAIYTTDDE

SEQ ID NO: 94 - protein, 23 aa, Artificial Sequence
  Description of Artificial Sequence Synthetic peptide
RPSNNRRKGIHMGPGKAIYTTDE

SEQ ID NO: 95 - protein, 25 aa, Artificial Sequence
  Description of Artificial Sequence Synthetic peptide
RPSNNNRRKGIHMGPGKAIYTTDEE

SEQ ID NO: 96 - protein, 23 aa, Artificial Sequence
  Description of Artificial Sequence Synthetic peptide
RPNNNTRKRMTLGPGKVFYTTGD

SEQ ID NO: 97 - protein, 24 aa, Artificial Sequence
  Description of Artificial Sequence Synthetic peptide
RPNNNTRKRMTLGPGKVFYTTGDE

SEQ ID NO: 98 - protein, 24 aa, Artificial Sequence
  Description of Artificial Sequence Synthetic peptide
RPNNNRRKRMTLGPGKVFYTTGDE

SEQ ID NO: 99 - protein, 24 aa, Artificial Sequence
  Description of Artificial Sequence Synthetic peptide
RPNNNRRKRMTLGPGKVFYTTGEE

SEQ ID NO: 100 - protein, 23 aa, Artificial Sequence
  Description of Artificial Sequence Synthetic peptide
RPNNNTRKRMTLGPGKVFYTTGD

SEQ ID NO: 101 - protein, 24 aa, Artificial Sequence
  Description of Artificial Sequence Synthetic peptide
RPNNNTRKRMTLGPGKVFYTTGDE

SEQ ID NO: 102 - protein, 24 aa, Artificial Sequence
  Description of Artificial Sequence Synthetic peptide
RPNNNRRKRMTLGPGKVFYTTGDE

SEQ ID NO: 103 - protein, 24 aa, Artificial Sequence
  Description of Artificial Sequence Synthetic peptide
RPNNNRRKRMTLGPGKVFYTTGEE

SEQ ID NO: 104 - protein, 23 aa, Artificial Sequence
  Description of Artificial Sequence Synthetic peptide
RPNNNTRKGIHIGPGRTFFATGD

SEQ ID NO: 105 - protein, 24 aa, Artificial Sequence
  Description of Artificial Sequence Synthetic peptide
RPNNNTRKGIHIGPGRTFFATGDE

SEQ ID NO: 106 - protein, 24 aa, Artificial Sequence
  Description of Artificial Sequence Synthetic peptide
RPNNNRRKGIHIGPGRTFFATGDE

SEQ ID NO: 107 - protein, 24 aa, Artificial Sequence
  Description of Artificial Sequence Synthetic peptide
RPNNNRRKGIHIGPGRTFFATGEE

SEQ ID NO: 108 - protein, 23 aa, Artificial Sequence
  Description of Artificial Sequence Synthetic peptide
RPNNNTRKGIHIGPGRTFFATGD

SEQ ID NO: 109 - protein, 24 aa, Artificial Sequence
  Description of Artificial Sequence Synthetic peptide
RPNNNTRKGIHIGPGRTFFATGDE

SEQ ID NO: 110 - protein, 24 aa, Artificial Sequence
  Description of Artificial Sequence Synthetic peptide
RPNNNRRKGIHIGPGRTFFATGDE

SEQ ID NO: 111 - protein, 24 aa, Artificial Sequence
  Description of Artificial Sequence Synthetic peptide
RPNNNRRKGIHIGPGRTFFATGEE

SEQ ID NO: 112 - protein, 23 aa, Artificial Sequence
  Description of Artificial Sequence Synthetic peptide
RPNNNTRKSIQIGPGRAFYTTGD

SEQ ID NO: 113 - protein, 24 aa, Artificial Sequence
  Description of Artificial Sequence Synthetic peptide
RPNNNTRKSIQIGPGRAFYTTGDE

SEQ ID NO: 114 - protein, 24 aa, Artificial Sequence
  Description of Artificial Sequence Synthetic peptide
RPNNNRRKSIQIGPGRAFYTTGDE

SEQ ID NO: 115 - protein, 25 aa, Artificial Sequence
  Description of Artificial Sequence Synthetic peptide
RPNNNTRRKSIQIGPGRAFYTTGEE

SEQ ID NO: 116 - protein, 23 aa, Artificial Sequence
  Description of Artificial Sequence Synthetic peptide
RPNNNTRKSIQIGPGRAFYTTGD

SEQ ID NO: 117 - protein, 24 aa, Artificial Sequence
  Description of Artificial Sequence Synthetic peptide
RPNNNTRKSIQIGPGRAFYTTGDE

SEQ ID NO: 118 - protein, 24 aa, Artificial Sequence
  Description of Artificial Sequence Synthetic peptide
RPNNNRRKSIQIGPGRAFYTTGDE

SEQ ID NO: 119 - protein, 25 aa, Artificial Sequence
  Description of Artificial Sequence Synthetic peptide
RPNNNTRRKSIQIGPGRAFYTTGEE

SEQ ID NO: 120 - protein, 39 aa, Artificial Sequence
  Description of Artificial Sequence Synthetic peptide
KQIINMWQEVGKAMYATRPNNNTRKSITKGPGRVIYATG

SEQ ID NO: 121 - protein, 39 aa, Artificial Sequence
  Description of Artificial Sequence Synthetic peptide
KQIINMWQGVGKAMYATRPNNNTRKSITKGPGRVIYATG

SEQ ID NO: 122 - protein, 39 aa, Artificial Sequence
  Description of Artificial Sequence Synthetic peptide
KQIINMWQVVGKAMYATRPNNNTRKSITKGPGRVIYATG

SEQ ID NO: 123 - protein, 40 aa, Artificial Sequence
  Description of Artificial Sequence Synthetic peptide
KQIIINMWQEVGEAMYATRPNNNTRKSITKGPGRVIYATG

SEQ ID NO: 124 - protein, 4 aa, Artificial Sequence
  Description of Artificial Sequence Synthetic peptide
RPNN

SEQ ID NO: 125 - protein, 4 aa, Artificial Sequence
  Description of Artificial Sequence Synthetic peptide
SITK

SEQ ID NO: 126 - protein, 4 aa, Artificial Sequence
  Description of Artificial Sequence Synthetic peptide
GPGR

SEQ ID NO: 127 - protein, 4 aa, Artificial Sequence
  Description of Artificial Sequence Synthetic peptide
GLGR

SEQ ID NO: 128 - protein, 4 aa, Artificial Sequence
  Description of Artificial Sequence Synthetic peptide
GPGG

SEQ ID NO: 129 - protein, 4 aa, Artificial Sequence
  Description of Artificial Sequence Synthetic peptide
GLGK

SEQ ID NO: 130 - protein, 4 aa, Artificial Sequence
  Description of Artificial Sequence Synthetic peptide
GLGL

SEQ ID NO: 131 - protein, 4 aa, Artificial Sequence
  Description of Artificial Sequence Synthetic peptide
GLGR

SEQ ID NO: 132 - protein, 21 aa, Artificial Sequence
  Description of Artificial Sequence Synthetic peptide
RPNNNTRRNIHIGLGRRFYAT

SEQ ID NO: 133 - protein, 22 aa, Artificial Sequence
  Description of Artificial Sequence Synthetic peptide
RPNNNTRRSVRIGPGGAMFRTG

SEQ ID NO: 134 - protein, 22 aa, Artificial Sequence
  Description of Artificial Sequence Synthetic peptide
RPIKIERKRIPLGLGKAFYTTK

SEQ ID NO: 135 - protein, 23 aa, Artificial Sequence
  Description of Artificial Sequence Synthetic peptide
RPSVNNTRRSIHMGLGRAFYTTG

SEQ ID NO: 136 - protein, 23 aa, Artificial Sequence
  Description of Artificial Sequence Synthetic peptide
RPNRHTGKSIRMGLGLRAWHTTR

SEQ ID NO: 137 - protein, 12 aa, Artificial Sequence
  Description of Artificial Sequence Synthetic peptide
RRNIHIGLGRRF

SEQ ID NO: 138 - protein, 12 aa, Artificial Sequence
  Description of Artificial Sequence Synthetic peptide
RRSVRIGPGGAM

SEQ ID NO: 139 - protein, 12 aa, Artificial Sequence
  Description of Artificial Sequence Synthetic peptide
RKSIRIGPGRAV

SEQ ID NO: 140 - protein, 12 aa, Artificial Sequence
  Description of Artificial Sequence Synthetic peptide
RRRISIGPGRAF

SEQ ID NO: 141 - protein, 12 aa, Artificial Sequence
  Description of Artificial Sequence Synthetic peptide
RKSIHIGPGRAF

SEQ ID NO: 142 - protein, 12 aa, Artificial Sequence
  Description of Artificial Sequence Synthetic peptide
RKSIHIAPGRAF

SEQ ID NO: 143 - protein, 22 aa, Artificial Sequence
  Description of Artificial Sequence Synthetic peptide
RPNNNTRKGIHIGPGRTFFATG

SEQ ID NO: 144 - protein, 22 aa, Artificial Sequence
  Description of Artificial Sequence Synthetic peptide
RPNNNTRKSINIGPGRAFYTTG

SEQ ID NO: 145 - protein, 22 aa, Artificial Sequence
  Description of Artificial Sequence Synthetic peptide
RPNNNTRKSIQIGPGRAFYTTG

SEQ ID NO: 146 - protein, 22 aa, Artificial Sequence
  Description of Artificial Sequence Synthetic peptide
RPNNNTRKSIHIGPGRAFYTTG

SEQ ID NO: 147 - protein, 22 aa, Artificial Sequence
  Description of Artificial Sequence Synthetic peptide
RPNNNTRKSIHIGPGRAFYATE

SEQ ID NO: 148 - protein, 22 aa, Artificial Sequence
  Description of Artificial Sequence Synthetic peptide
RPNNNTRKRMTLGPGKVFYTTG

SEQ ID NO: 149 - protein, 22 aa, Artificial Sequence
  Description of Artificial Sequence Synthetic peptide
RPGNNTRGSIHLHPGRKFYYSR

SEQ ID NO: 150 - protein, 13 aa, Artificial Sequence
  Description of Artificial Sequence Synthetic peptide
IHIGPGRAFYTTG

SEQ ID NO: 151 - protein, 45 aa, Artificial Sequence
  Description of Artificial Sequence Synthetic peptide
SVEINCTRPNNNTRKSIHIGPGRAFYTTGEIIGDIRQAHCNISRA

SEQ ID NO: 152 - protein, 45 aa, Artificial Sequence
  Description of Artificial Sequence Synthetic peptide
SVEINCTRPNNNTRKSIHIGPGRAFYATERIIGDIRQAHCNISRT

SEQ ID NO: 153 - protein, 47 aa, Artificial Sequence
  Description of Artificial Sequence Synthetic peptide
SVEINCTRPNNNTRKSIHIGPGRAFYATETTRIIGDIRQAHCNISRT

SEQ ID NO: 154 - protein, 45 aa, Artificial Sequence
  Description of Artificial Sequence Synthetic peptide
SVEINCTRPGNNTRGSIHLHPGRKFYYSRGIIGDIREAHCAINIP

SEQ ID NO: 155 - protein, 45 aa, Artificial Sequence
  Description of Artificial Sequence Synthetic peptide
SVEINCTRPNNNTRRSVRIGPGGAMFRTGDIIGDIRQAHCNLSRT

SEQ ID NO: 156 - protein, 45 aa, Artificial Sequence
  Description of Artificial Sequence Synthetic peptide
SIEINCTRPNNNTRKSIQIGPGRAFYTTGEIIGDIRQAHCNLSRA

SEQ ID NO: 157 - protein, 45 aa, Artificial Sequence
  Description of Artificial Sequence Synthetic peptide
SVEINCTRPNNNTRKRMTLGPGKVFYTTGEIIGDIRKAHCNISRA

SEQ ID NO: 158 - protein, 44 aa, Artificial Sequence
  Description of Artificial Sequence Synthetic peptide
SVAINCTRRNNNTRRNIHIGLGRRFYATEIIGDTKKADCNISRA

SEQ ID NO: 159 - protein, 39 aa, Artificial Sequence
  Description of Artificial Sequence Synthetic peptide
SVEINCTRPIKIERKRIPLGLGKAFYTTKQVGDIKQAHC

SEQ ID NO: 160 - protein, 45 aa, Artificial Sequence
  Description of Artificial Sequence Synthetic peptide
PVEINCTRPNNNTRRSIHIAPGRAFYTTGQIIGDIRRAHCNISRT

SEQ ID NO: 161 - protein, 45 aa, Artificial Sequence
  Description of Artificial Sequence Synthetic peptide
TVVINCTRPNRHTGKSIRMGLGRAWHTTREIIGDIRKAYCTLNGT

SEQ ID NO: 162 - protein, 45 aa, Artificial Sequence
  Description of Artificial Sequence Synthetic peptide
SVNINCTRPNNNTRKGIHIGPGRTFFATGDIIGDIRQAHCNLSRT

SEQ ID NO: 163 - protein, 36 aa, Artificial Sequence
  Description of Artificial Sequence Synthetic peptide
CTRPNNNTRKSIHIGPGRAFYTTGEIIGDIRIQAHC

SEQ ID NO: 164 - protein, 15 aa, Artificial Sequence
  Description of Artificial Sequence Synthetic peptide
  MOD_RES (1)..(2): Variable amino acid
  MOD_RES (14)..(15): Variable amino acid
  see specification as filed for detailed description of substitutions and preferred embodiments
XXSIHIGPGRAFYXX

SEQ ID NO: 165 - protein, 15 aa, Artificial Sequence
  Description of Artificial Sequence Synthetic peptide
  MOD_RES (1)..(2): Variable amino acid
  MOD_RES (14)..(15): Variable amino acid
  see specification as filed for detailed description of substitutions and preferred embodiments
XXSINIGPGRAFYXX

SEQ ID NO: 166 - protein, 15 aa, Artificial Sequence
  Description of Artificial Sequence Synthetic peptide
  MOD_RES (1)..(2): Variable amino acid
  MOD_RES (14)..(15): Variable amino acid
  see specification as filed for detailed description of substitutions and preferred embodiments
XXGIHMGPGKAIYXX

SEQ ID NO: 167 - protein, 14 aa, Artificial Sequence
  Description of Artificial Sequence Synthetic peptide
  MOD_RES (1)..(2): Variable amino acid
  MOD_RES (13)..(14): Variable amino acid
  see specification as filed for detailed description of substitutions and preferred embodiments
XXMTLGPGKVFYXX

SEQ ID NO: 168 - protein, 15 aa, Artificial Sequence
  Description of Artificial Sequence Synthetic peptide
  MOD_RES (1)..(2): Variable amino acid
  MOD_RES (14)..(15): Variable amino acid
  see specification as filed for detailed description of substitutions and preferred embodiments
XXGIHIGPGRTFFXX

SEQ ID NO: 169 - protein, 15 aa, Artificial Sequence
  Description of Artificial Sequence Synthetic peptide
  MOD_RES (1)..(2): Variable amino acid
  MOD_RES (14)..(15): Variable amino acid
  see specification as filed for detailed description of substitutions and preferred embodiments
XXSIQIGPGRAFYXX

SEQ ID NO: 170 - protein, 39 aa, Artificial Sequence
  Description of Artificial Sequence Synthetic peptide
KQIINMWQEVGKAMYATRPHNNTRKSIHMGPGKAFYTTG

SEQ ID NO: 171 - protein, 35 aa, Artificial Sequence
  Description of Artificial Sequence Synthetic peptide
CTRPHNNTRKSIHMGPGKAFYTTGGIIGDIRQAHC

SEQ ID NO: 172 - protein, 28 aa, Artificial Sequence
  Description of Artificial Sequence Synthetic peptide
CRIKQIINMWQEVGKAMYAPPISKQIRC

SEQ ID NO: 173 - protein, 39 aa, Artificial Sequence
  Description of Artificial Sequence Synthetic peptide
KQIINMWQEVGKAMYATRPNYNKRKRIHIGPGRAFYTTK

SEQ ID NO: 174 - protein, 39 aa, Artificial Sequence
  Description of Artificial Sequence Synthetic peptide
KQIINMWQEVGKAMYATRPGNNTRKSIPIGPGRAFIATS

SEQ ID NO: 175 - protein, 39 aa, Artificial Sequence; identical to SEQ ID NO: 170
  Description of Artificial Sequence Synthetic peptide

SEQ ID NO: 176 - protein, 10 aa, Artificial Sequence
  Description of Artificial Sequence Synthetic peptide
RPNNNTRKSI

SEQ ID NO: 177 - protein, 35 aa, Artificial Sequence
  Description of Artificial Sequence Synthetic peptide
CTRPNNNTRKGIHIGPGGAFYATGDIIGDIRQAHC

SEQ ID NO: 178 - protein, 35 aa, Artificial Sequence
  Description of Artificial Sequence Synthetic peptide
  MOD_RES (32): Variable amino acid
CTRPNNNTRKSVTLGPGRVWYTTGQIVGDIRXAHC

SEQ ID NO: 179 - protein, 35 aa, Artificial Sequence
  Description of Artificial Sequence Synthetic peptide
  MOD_RES (22): Variable amino acid
CTRPSNNTRKSVHMGWRRTFFXTEKIIGDVRKAHC

SEQ ID NO: 180 - protein, 35 aa, Artificial Sequence
  Description of Artificial Sequence Synthetic peptide
  MOD_RES (10): Variable amino acid
  MOD_RES (26): Variable amino acid
CSRPNNNTRXSVRIGPGQTFYATGDXIGDIRQAHC

SEQ ID NO: 181 - protein, 35 aa, Artificial Sequence
  Description of Artificial Sequence Synthetic peptide
  MOD_RES (35): Variable amino acid
CTRPNNNTSKSIHMGAGRAWHATRKIIGDIKKAHX

SEQ ID NO: 182 - protein, 35 aa, Artificial Sequence
  Description of Artificial Sequence Synthetic peptide
  MOD_RES (4): Variable amino acid
  MOD_RES (25): Variable amino acid
  MOD_RES (27): Variable amino acid
CTRXHNSKRTRIRIGPGRSYYTTKXIXGPIGQAHC

SEQ ID NO: 183 - protein, 35 aa, Artificial Sequence
  Description of Artificial Sequence Synthetic peptide
  MOD_RES (23): Variable amino acid
CTRPSNNTRKGIYIGPGRKVYTXEKITGDMKKAYC

SEQ ID NO: 184 - protein, 35 aa, Artificial Sequence
  Description of Artificial Sequence Synthetic peptide
  MOD_RES (27): Variable amino acid
CTRPSNHTQRRIAIGPGRSFYATDRIXGDLKQAHC

SEQ ID NO: 185 - protein, 35 aa, Artificial Sequence
  Description of Artificial Sequence Synthetic peptide
  MOD_RES (26): Variable amino acid
CIRPNNYSAKAIRIGPGRAVIATKRXIGNIRQAHC

SEQ ID NO: 186 - protein, 35 aa, Artificial Sequence
  Description of Artificial Sequence Synthetic peptide
CTRPNNNTRKSIHIGPGRAFYATGEVIGDIRQAHC

SEQ ID NO: 187 - protein, 35 aa, Artificial Sequence
  Description of Artificial Sequence Synthetic peptide
  MOD_RES (20): Variable amino acid
  MOD_RES (22): Variable amino acid
CTRPNNNTRKSIHIGPGRAXYXTGEIIGDIRQAHC

SEQ ID NO: 188 - protein, 35 aa, Artificial Sequence
  Description of Artificial Sequence Synthetic peptide
CTRPNNNTRKSIHIGPGRAFYTTGEIIGDIRQAHC

SEQ ID NO: 189 - protein, 35 aa, Artificial Sequence
  Description of Artificial Sequence Synthetic peptide
  MOD_RES (14): Variable amino acid
  MOD_RES (19): Variable amino acid
CTRPNNNTRKSITXGPGRXFYATGDIIGDIRQAHC

SEQ ID NO: 190 - protein, 35 aa, Artificial Sequence
  Description of Artificial Sequence Synthetic peptide
CTRPNNNTRRSIHIGPGRAFYATGDIIGDIRQAHC

SEQ ID NO: 191 - protein, 35 aa, Artificial Sequence
  Description of Artificial Sequence Synthetic peptide
CTRPNNNTRKSISIGPGRAFYATGDIIGDIRQAHC

SEQ ID NO: 192 - protein, 35 aa, Artificial Sequence
  Description of Artificial Sequence Synthetic peptide
CTRPNNNTRKSIHMGPGAAFYARGEVIGDIRQAHC

SEQ ID NO: 193 - protein, 35 aa, Artificial Sequence
  Description of Artificial Sequence Synthetic peptide
CTRPGNNTRKSIHIGPGRAFYATGDIIGDIRQAHC

SEQ ID NO: 194 - protein, 35 aa, Artificial Sequence
  Description of Artificial Sequence Synthetic peptide
CTRPNNNTRKSIHMGPGKVFYTTGEIIGDIRQAHC

SEQ ID NO: 195 - protein, 35 aa, Artificial Sequence
  Description of Artificial Sequence Synthetic peptide
CERPNNNTIKSIHLGPGRAWHATGQIIGDIRKAFC

SEQ ID NO: 196 - protein, 34 aa, Artificial Sequence
  Description of Artificial Sequence Synthetic peptide
CTRPNNNTRKSIHIGAGKALYTGEIIGDIRQAHC

SEQ ID NO: 197 - protein, 22 aa, Artificial Sequence
  Description of Artificial Sequence Synthetic peptide
RPNNNTRRSIHLGMGRALYATG

SEQ ID NO: 198 - protein, 22 aa, Artificial Sequence
  Description of Artificial Sequence Synthetic peptide
RPSNNTRRSIHMGLGRAFYTTG

SEQ ID NO: 199 - protein, 22 aa, Artificial Sequence
  Description of Artificial Sequence Synthetic peptide
RPNRHTGKSIRMGLGRAWHTTR

SEQ ID NO: 200 - protein, 22 aa, Artificial Sequence
  Description of Artificial Sequence Synthetic peptide
RPNNNTRKRISLGPGRVYYTTG

SEQ ID NO: 201 - protein, 24 aa, Artificial Sequence
  Description of Artificial Sequence Synthetic peptide
RPNYNETKRIRIHRGYGRSFVTVR

SEQ ID NO: 202 - protein, 21 aa, Artificial Sequence
  Description of Artificial Sequence Synthetic peptide
RPDNNTVRKIPIGPGSSFYTT

SEQ ID NO: 203 - protein, 22 aa, Artificial Sequence
  Description of Artificial Sequence Synthetic peptide
RPSNNTRKGIHLGPGRAIYATE

SEQ ID NO: 204 - protein, 22 aa, Artificial Sequence
  Description of Artificial Sequence Synthetic peptide
RPNNNTRRRISMGPGRVLYTTG

SEQ ID NO: 205 - protein, 22 aa, Artificial Sequence
  Description of Artificial Sequence Synthetic peptide
RPGNNTRGSIHLHPGRKFYYSR

SEQ ID NO: 206 - protein, 22 aa, Artificial Sequence
  Description of Artificial Sequence Synthetic peptide
RPDNTIKQRIIHIGPGRPFYTT

SEQ ID NO: 207 - protein, 22 aa, Artificial Sequence
  Description of Artificial Sequence Synthetic peptide
RPNNNTRKSINMGPGRAFYTTG

SEQ ID NO: 208 - protein, 22 aa, Artificial Sequence
  Description of Artificial Sequence Synthetic peptide
RPNNNTRRSIPIGPGRAFYTTG

SEQ ID NO: 209 - protein, 22 aa, Artificial Sequence
  Description of Artificial Sequence Synthetic peptide
RPGNTTRRGIPIGPGRAFFTTG

SEQ ID NO: 210 - protein, 22 aa, Artificial Sequence
  Description of Artificial Sequence Synthetic peptide
RPNNNTRKSIRIGPGRAVYATD

SEQ ID NO: 211 - protein, 22 aa, Artificial Sequence
  Description of Artificial Sequence Synthetic peptide
RPNNNTRKGINIGPGRAFYATG

SEQ ID NO: 212 - protein, 25 aa, Artificial Sequence
  Description of Artificial Sequence Synthetic peptide
RPNNNTRKRIRIGHIGPGRAFYATG

SEQ ID NO: 213 - protein, 21 aa, Artificial Sequence
  Description of Artificial Sequence Synthetic peptide
RPSINKRRHIHIGPGRAFYAT

SEQ ID NO: 214 - protein, 22 aa, Artificial Sequence
  Description of Artificial Sequence Synthetic peptide
RLYNYRRKGIHIGPGRAIYATG

SEQ ID NO: 215 - protein, 22 aa, Artificial Sequence
  Description of Artificial Sequence Synthetic peptide
RPNNNTRKSIHIAPGRAFYTTG

SEQ ID NO: 216 - protein, 22 aa, Artificial Sequence
  Description of Artificial Sequence Synthetic peptide
RPSNNTRKGIHMGPGKAIYTTD

SEQ ID NO: 217 - protein, 22 aa, Artificial Sequence
  Description of Artificial Sequence Synthetic peptide
RPNNNTRKSIHIGPGRAFYTTG

SEQ ID NO: 218 - protein, 22 aa, Artificial Sequence
  Description of Artificial Sequence Synthetic peptide
RPNNNTRKSINIGPGRAFYTTG

SEQ ID NO: 219 - protein, 22 aa, Artificial Sequence
  Description of Artificial Sequence Synthetic peptide
RPNNNTRKRMTLGPGKVFYTTG

SEQ ID NO: 220 - protein, 22 aa, Artificial Sequence
  Description of Artificial Sequence Synthetic peptide
RPNNNTRKGIHIGPGRTFFATG

SEQ ID NO: 221 - protein, 22 aa, Artificial Sequence
  Description of Artificial Sequence Synthetic peptide
RPNNNTRKSIQIGPGRAFYTTG

SEQ ID NO: 222 - protein, 22 aa, Artificial Sequence
  Description of Artificial Sequence Synthetic peptide
RPNNNTRKSIHIGPGRAFYATE

SEQ ID NO: 223 - protein, 12 aa, Artificial Sequence
  Description of Artificial Sequence Synthetic peptide
KRIHIGPGRAFY

SEQ ID NO: 224 - protein, 36 aa, Artificial Sequence; identical to SEQ ID NO: 163
  Description of Artificial Sequence Synthetic peptide

SEQ ID NO: 225 - protein, 34 aa, Artificial Sequence
  Description of Artificial Sequence Synthetic peptide
CTRPNNNTRKSIHIGPGRAFYTTGEIGDIRQAHC

SEQ ID NO: 226 - protein, 23 aa, Artificial Sequence
  Description of Artificial Sequence Synthetic peptide
TRPNNNTRKSIHIGPGRAFYTTG

SEQ ID NO: 227 - protein, 23 aa, Artificial Sequence
  Description of Artificial Sequence Synthetic peptide
TRPNNNTRKSIHIGPGRAFYATE

SEQ ID NO: 228 - protein, 23 aa, Artificial Sequence
  Description of Artificial Sequence Synthetic peptide
TRPNNNTRKGIHIGPGRTFFATG

SEQ ID NO: 229 - protein, 23 aa, Artificial Sequence
  Description of Artificial Sequence Synthetic peptide
TRPNNNTRKSIQIGPGRAFYTTG

SEQ ID NO: 230 - protein, 24 aa, Artificial Sequence
  Description of Artificial Sequence Synthetic peptide
TRPNRHTGKSIRMGLGLRAWHTTR

SEQ ID NO: 231 - protein, 23 aa, Artificial Sequence
  Description of Artificial Sequence Synthetic peptide
TRPNYNKRKRIHIGPGRAFYTTK

SEQ ID NO: 232 - protein, 35 aa, Artificial Sequence; identical to SEQ ID NO: 188
  Description of Artificial Sequence Synthetic peptide

SEQ ID NO: 233 - protein, 35 aa, Artificial Sequence
  Description of Artificial Sequence Synthetic peptide
CTRPNNNTRRRLSIGPGRAFYARRNIIGDIRQAHC

SEQ ID NO: 234 - protein, 34 aa, Artificial Sequence
  Description of Artificial Sequence Synthetic peptide
CTRPNNNTRRRLSIGPGRAFYARRNIIGDIRQAH

SEQ ID NO: 235 - protein, 21 aa, Artificial Sequence
  Description of Artificial Sequence Synthetic peptide
KRKRIHIGPGRAFYTTKNIIG

SEQ ID NO: 236 - protein, 36 aa, Artificial Sequence; identical to SEQ ID NO: 163
  Description of Artificial Sequence Synthetic peptide

SEQ ID NO: 237 - protein, 35 aa, Artificial Sequence; identical to SEQ ID NO: 188
  Description of Artificial Sequence Synthetic peptide

(19) United States
(12) Patent Application Publication
(10) Pub. No.: US 2008/0181906 A1
(43) Pub. Date: Jul. 31, 2008

(54) Polyvalent Immunogen

(76) Inventors: Barton F. Haynes, Durham, NC (US); Leonard D. Spicer, Durham, NC (US)

Correspondence Address: NIXON & VANDERHYE, PC, 901 NORTH GLEBE ROAD, 11TH FLOOR, ARLINGTON, VA, 22203, US

(21) Appl. No.: 10/557,467

(22) PCT Filed: May 19, 2004
(86) PCT No.: PCT/US04/15735

§ 371 Date: Jan. 3, 2007

Related U.S. Application Data
(60) Provisional application No. 60/471,327, filed on May 19, 2003.

(51) Int. Cl. A61K 31/70; A61K 38/00; A61P 43/00; C07H 21/04; C07K 14/00; C12N 15/00

(52) U.S. Cl. 424/185.1; 435/320.1; 514/44; 530/403; 536/23.5

(57) ABSTRACT

The present invention relates, generally, to a polyvalent immunogen and, more particularly, to a method of inducing neutralizing antibodies against HIV and to a polyvalent immunogen suitable for use in such a method.

This application claims priority from Provisional Application No. 60/471,327, filed May 19, 2003, the content of which is incorporated by reference.

### TECHNICAL FIELD

The present invention relates, generally, to a polyvalent immunogen and, more particularly, to a method of inducing neutralizing antibodies against HIV and to a polyvalent immunogen suitable for use in such a method.

### BACKGROUND

Immunogenic peptides have been developed that elicit B and T cell responses to various strains of human immunodeficiency virus (HIV) (Palker et al, J. Immunol. 142:3612-3619 (1989), Haynes et al, Trans. Am. Assoc. Physician 106:31-41 (1993), Haynes et al, J. Immunol. 151:1646-1653 (1993), Haynes et al, AID Res. Human Retroviruses 11:211-221 (1995)) (see also WO 97/14436). These peptides consist of two components, each derived from noncontiguous regions of the HIV gp120 envelope protein. One envelope component consists of 16 amino acid residues from the fourth constant (C4) domain of HIV gp120, and includes a T-helper epitope (Cease et al, Proc. Natl. Acad. Sci. USA 84:4249-4253 (1987)). Linked to the carboxyl terminus of this gp120 C4 region peptide is a 23 amino acid segment from the third variable (V3) domain of gp120, that includes a B cell neutralizing antibody epitope for cell line-adapted HIV strains (Palker et al, J. Immunol. 142:3612-3619 (1989), (Palker et al, Proc. Natl. Acad. Sci. USA 85:1932-1936 (1988), Ruscbe et al, Proc. Natl. Acad. Sci. USA 85:3198-3202)), a T-helper epitope (Palker et al, J. Immunol. 142:3612-3619 (1989)), and two cytotoxic T lymphopoietic (CTL) epitopes (Clerici et al, J. Immunol. 146:2214-2219 (1991), Safrit et al, 6^{th} NCVDG Meeting, Oct. 30 to Nov. 4, 1993)). In mice and rhesus monkeys, these C4-V3 hybrid peptides have induced antibodies that bind to native gp120 and neutralize the particular cell line-adapted strain of HIV from which the V3 segment was derived, as well as induce T helper cell proliferative responses and MHC Class I-restricted CTL responses that kill HIV or HIV protein expressing target cells (Palker et al, J. Immunol. 142:3612-3619 (1989), Haynes et al, AID Res. Human Retroviruses 11:211-221 (1995)). Recently, it was shown that this gp120 peptide design can induce antibodies that neutralize primary HIV isolates and simian-human immunodeficiency viruses (SHIV) expressing primary HIV isolate envelopes (Liao et al, J. Virol. 74:254-263 (2000)). Moreover, in a challenge trial of this immunogen in rhesus monkeys, it was shown that C4-V3 peptides from the gp120 of the pathogenic SHIV 89.6P, induced neutralizing antibodies that prevented the fall in CD4 counts after challenge with SHIV 89.6P (Letvin et al, J. Virol. 75:4165-4175 (2001)). Therefore, anti-V3 antibodies can protect primates against primary isolate SHIV-induced disease.

A prototype polyvalent HIV experimental immunogen comprised of the conserved C4 region of gp120 and the V3 regions of HIV isolates MN, CANO(A), EV91 and RF has been constructed and has been found to be highly immunogenic in human clinical trials (Bartlett et al, AIDS 12:1291-1300 (1998), Graham et al, Abstract, AIDS Vaccine (2001)). Thus, understanding secondary and higher order structures of the components of this polyvalent immunogen, as well as defining strategies to optimize gp120 immunogen antigenicity, is important to HIV vaccine design efforts. In addition, recent data suggest that the HIV V3 region may be involved in regulating gp120 interactions with HIV co-receptors, CXC chemokine receptor 4 (CXCR4) and chemokine receptor type 5 (CCR5) (Berger, AIDS Suppl. A:53-56 (1997)).

In previous studies, nuclear magnetic resonance (NMR) has been used to characterize conformations of the multivalent immunogen C4-V3 peptides in solution (de Lorimier et al, Biochemistry 33:2055-2062 (1994), Vu et al, Biochemistry 35:5158-5165 (1996), Vu et al, J. Virol. 73:746-750 (1999)). It as been found that the V3 segments of each of the four C4-V3 peptides displayed evidence of preferred solution conformations, with some. features shared, and other features differing among the four peptides. The C4 segment, which is of identical sequence in all the peptides, showed in each case a tendency to adopt nascent helical conformations (de Lorimier et al, Biochemistry 33:2055-2062 (1994), Vu et al, Biochemistry 35:5158-5165 (1996), Vu et al, J. Virol. 73:746-750 (1999)).

The C4 sequence as a peptide does not elicit antibodies that bind native gp120 (Palker et al, J. Immunol. 142:3612-3619 (1989), Haynes et al, J. Immunol. 151:1646-1653 (1993), Ho et al, J. Virol. 61:2024-2028 (1987), Robey et al, J. Biol. Chem. 270:23918-23921 (1995)). This led to the speculation that the nascent helical conformations exhibited by the C4 segment might reflect a conformation not native to HIV gp120. Amino-acid sequence homology between the gp120 C4 region and a human IgA CHI domain has been noted (Maddon et al, Cell 47:333-348 (1986)). By comparison to the structure of mouse IgA (Segal et al, Proc. Natl. Acad. Sci. USA 71:4298-4302 (1974)), the C4-homologous region of IgA has a β strand secondary structure (de Lorimier et al, Biochemistry 33:2055-2062 (1994)). Therefore, while the C4 gp120 peptide in solution adopts nascent helical conformations, the native structure of this gp120 C4 region may be quite different (ie, in the context of gp 120 have a β strand secondary structure).

The present invention results, at least in part, from the results of a study with a three-fold purpose. First, C4-V3HIVRF peptides with amino acid substitutions designed to minimize C4 α-helical peptide conformation and promote β strand C4 secondary structures were constructed in order to induce anti-native gp120 antibodies with the modified C4 peptide. Second, tests were made to determine if any of these mutated C4-V3RF peptides would enhance gp120 V3 region peptide immunogenicity, and therefore augment anti-HIVRF gp120 V3 loop antibody responses. Finally, the solution conformers of each peptide studied immunologically were also solved using NMR to correlate peptide conformers with peptide immunogenicity.

### SUMMARY OF THE INVENTION

The present invention relates to a method of inducing neutralizing antibodies against HIV and to peptides, and DNA sequences encoding same, that are suitable for use in such a method.

In one embodiment, the invention relates to a composition comprising a multiplicity of immunogenic peptides comprising a first and a second component, the first component comprising a T-helper epitope, the second component comprising residues of the V3 domain of gp 120 and including a B cell neutralizing antibody epitope. The first component can be a human immunodeficiency virus (HIV) T helper epitope. The first component can comprise residues of the C4 domain of HIV gp120, for example, at least 16 contiguous residues of the C4 domain of HIV gp120 (e.g., residues 421 to 436 of the C4 domain of HIV gp120). The first component can comprise residues of HIV p24 gag (e.g., GTH1 (residues 262-278 of HIV gag)). The first component can be a non-HIV T helper epitope. The second component can comprise at least 23 contiguous residues of the V3 domain of HIV gp120 (e.g., residues 297 to 322 of the V3 domain of HIV gp120)). The first component can comprise at least 16 contiguous residues of the C4 domain of HIV gp120 and the second component can comprise at least 23 contiguous residues of the V3 domain of HIV gp120 (e.g., residues 421 to 436 of the C4 domain of HIV gp120 and residues 297 to 322 of the V3 domain of HIV gp120). The second component can be linked C terminal to the first component. The first component can be linked to the second component via a linker. The composition can comprise at least 5 immunogenic peptides (e.g., C4-V3 36.29, C4-V3 34.29, C4-V3 62.19, C4-V3 74.17 and C4-V3 162.7 from Table 7). The composition can comprise at least 10, or at least 25 immunogenic peptides. The composition can further comprise a carrier and/or an adjuvant. The first component can comprise the sequence YKRWIILGLNKIVRM. The second components can be selected so as to be representative of higher order structural motifs present in a population, which motifs mediate V3 functions in the course of envelope mediated HIV interaction with host cells. The composition can comprise about 25-30 immunogenic peptides the second components of which are selected so as to be representative of infected individuals within a subtype. At least one of the first components can comprise the sequence KQIINMWQVVGKAMYA. This aspect of the invention further relates to a method of inducing the production of neutralizing antibodies in a patient comprising administering to the patient an amount of the above composition sufficient to effect the production. In another aspect of this embodiment, the invention relates to a formulation comprising at least one nucleic acid sequence encoding the above composition and to a method of inducing the production of neutralizing antibodies in a patient comprising administering to the patient an amount of the formulation sufficient to effect the production.

In another embodiment, the invention relates to a composition comprising at least one peptide from Table 6 or Table 7 and a carrier. This composition can further comprise an adjuvant. In another embodiment, the invention relates to a nucleic acid encoding a peptide in Table 6 or Table 7.

In a further embodiment, the invention relates to an isolated polypeptide comprising a V3 sequence shown in Table 10, 11 or 12 (e.g., polypeptide 62.19) and to an isolated nucleic acid sequence encoding at least one such polypeptide. The invention further relates to a vector comprising such a nucleic acid. Additionally the invention relates to a method of inducing the production of neutralizing antibodies in a mammal comprising administering to the mammal an amount of such a polypeptide or nucleic acid sequence sufficient to effect the induction (e.g., the V3 sequence can be administered in a DNA prime with the V3 sequence in a gp140 or gp160 boost (or a gp120, gp140 or gp160 replication vector boost) (a replicating vector comprising the V3 sequence in envelope can be administered as prime and boost). The invention further relates to a composition comprising such a polypeptide or nucleic acid sequence and a carrier.

Other embodiment, objects and advantages of the present invention will be clear from the description that follows.

### BRIEF DESCRIPTION OF THE DRAWINGS

FIG. 1: Summary of antibody binding titers to immunizing peptide after 2 or 3 boosts of 3 mice in each group with immunizing peptide. There was a slight enhancement of levels of antibody induced by the E9G variant after 2 but not 3 boosts, while the E9V variant significantly boosted antibody levels compared to the C4-V3RF(A) peptide after 2 and 3 boosts. Antibody to the K12E variant induced by the K12E peptide was significantly lower than C4-V3RF(A:) induced antibody levels after both 2 and 3 boosts.

FIGS. 2A, 2B, 2C, 2D: NMR spectra of the four C4-V3RF variant peptides.

FIGS. 3A, 3B, 3C, 3D: C4_{E9V}-V389.6 peptides bound better to human PB lymphocytes and monocytes than did the C4-V3 89.6 peptides. Similar data were obtained with the C4-V3 89.6P and C4-E9V-89.6P peptides. Sequence of the C4-V389.6 peptide form HIV89.6 isolate was: KQIINMWQEVGKAMYA-TRPNNNTRRRLSIGPGRAFYARR; the sequence of the C4_{E9V}-V389.6 peptide was: KQIINMWQVVGKAMYA-TRPNNNTRRRLSIGPGRAFYARR; the sequence of the C4-V389.6P peptide was: KQIINMWQEVGKAMYA-TRPNNNTRERLSIGPGRAFYARR; the sequence of the C4E9V-V389.6P peptide was: KQIINMWQVVGKAMYA-TRPNNNTRERLSIGPGRAFYARR.

FIG. 4: Neutralization of BAL in PBMC.

FIG. 5: Neutralization of HIV primary isolates by sera from guinea pig (GP) 469 immunized with the C4-V3 peptide 62.19. The isolates tested are listed on the right side. The grey and white areas indicate no neutralization. The red boxes indicate >50% neutralization. The titers are 1:10, 1:30, 1:90 and 1:270 going across in each column.

FIG. 6: C4-V3 sequences tested.

FIG. 7: Strategy for design of HIV gp120 immunogens with higher order structures.

### DETAILED DESCRIPTION OF THE INVENTION

The present invention relates, at least in part, to a composition comprising a multiplicity of immunogenic hybrid peptides, each comprising two components. One component includes a T-helper epitope and can comprise residues from the C4 domain of HIV gp120. The second component comprises residues from the V3 domain of gp120 and includes a B cell neutralizing antibody epitope.

Advantageously, the first component comprises about 16 contiguous residues from the C4 domain (about residues 421 to 436) and the second component comprises about 23-25 contiguous residues from the V3 domain (about residues 297 to 0.322). The components can, however, be longer, and can comprise, for example, the entirety of the cysteine to cysteine V3 loop region, or be shorter. Preferably, the V3 component is linked C terminal to the C4 component peptide. The hybrid peptides can include additional sequences (e.g., linkers (e.g., cysteine, serine or lysine linkers) between the C4 and V3 components). The composition can, for example, comprise 5 to 10 hybrid peptides, 10 to 15 hybrid peptides or 25 to 30 hybrid peptides. The number of hybrid peptides used will depend, at least in part, on the target population.

Preferred first components comprising residues from the C4 domain are shown in the Tables that follow (see particularly Tables 6 and 7). Other T helper determinants from HIV or from non-HIV proteins can also be used.

For example, a further T helper epitope suitable for use in the invention is from HIV gag (e.g., residues 262-278). One such sequence, designated GTH1, is YKRWIILGLNKIVRMYS (from HIV p24 gag). Variants of this sequence can also be used. Alternatively, or in addition, a carbohydrate such as the outer membrane protein of pneumococcus, or another carbohydrate or protein with immunogenic, T helper activity can be used.

The V3 components of the hybrid peptides present in the instant composition are selected so as to be representative of higher order structural motifs present in a population, which motifs mediate V3 functions in the course of envelope mediated HIV interaction with host cells. The Los Alamos National Laboratories Human Retroviruses and AIDS Database (Human Retroviruses and ADS, 2000, Published by the Theoretical Biology and Biophysics G T-10, Mail Stop K710, LANL, Los Alamos. N. Mex.) presently contains over 14,000 HIV V3 envelope sequences, showing the extraordinary diversity the virus has obtained since originating in man in Africa approximately 50 years ago. For example, among 432 HIV-1 V3 sequences derived from individuals infected with subtype C (designated “Clade C”) in Africa currently available in the HIV database, 176 distinct variants of a 23 amino acid stretch at the tip of the V3 loop have been found. Similarly, among 6870 B subtype (designated “Clade B”) V3 sequences from the US, 1514 unique forms have been found.

A method has been developed to organize short antigenic domains by protein similarity scores using maximum-linkage clustering. This method enables the visualization of the clustering patterns as a dendrogram, and the splitting patterns in the dendrogram can be used to define clusters of related sequences (Korber et al, J. Virol. 68:6730-6744 (1994)). The method allows the use of several different amino acid similarity scoring schemes available in the literature, preferred is the amino acid substitution matrix developed by Henikoff and Henikoff (see Advances in Protein Chemistry 54:73-97 (2000) and Proteins: Structure, Function and Genetics 17:49-61 (1993)), designed to give substitutions that are-well tolerated in conserved protein structural elements a high score, and a low score to those that are not. Typically excluded from consideration very rare, highly divergent peptides, and favored are peptides found in many individuals within the population. In a selected set of sequences, most of the unique forms are within one or two amino acids from a least one other of the peptides chosen. This method has been applied to clustering the large number of variants of the antigenic tip of the V3 domain within Clade B and Clade C into groups (about 25) that are likely to be cross-reactive within the group. Based on these clustering patterns, variants (e.g., about 25-30) are selected that are representative or “central” to each group, for testing for antigenicity. The HIV Clade B and Clade C gp120 envelope V3 sequences have been analyzed, as described above, for groups of V3 sequences predicted to have structural similarities. Twenty five Clade C and 30 Clade B groups have been defined, and chosen out of each group is a common, or the most common, sequence as a representative of that group. The selected V3 sequences have been included in a C4-V3 design thereby providing a 25 peptide Clade C immunogen, and a 30 peptide Clade B immunogen (see Tables 6 and 7).

TABLE 6
C4-V3 design of Clade C V3 sequences

| | |
|---|---|
| C4-V3-C1 | KQIINMWQVVGKAMYA-trpnnntrksirigpGqtfyatg |
| C4-V3-C2 | KQIINMWQVVGKAMYA-trpnnntrksingpGqtfyaRg |
| C4-V3-C3 | KQIINMWQVVGKAMYA-trpnnntrksirigpGqtfyaAg |
| C4-V3-C4 | KQIINMWQVVGKAMYA-IrpnnntrksVrigpGqtfyatg |
| C4-V3-C5 | KQIINMWQVVGKAMYA-trpnnntrksirigpGqtfFatg |
| C4-V3-C6 | KQIINMWQVVGKAMYA-trpnnntrksirigpGqtfyatN |
| C4-V3-C7 | KQLINMWQVVGKAMYA-trpnnntrEsirigpGqtfyatg |
| C4-V3-C8 | KQIINMWQVVGKAMYA-trpnnntrRsirigpGqAfyatg |
| C4-V3-C9 | KQIINMWQVVGKAMYA-trpnnntrkGirigpGqtfyatg |
| C4-V3-C10 | KQIINMWQVVGKAMYA-trpSnntrksirigpGqAfyatg |
| C4-V3-C11 | KQIINMWQVVGKAMYA-trpSnntrksirigpGqtfyatN |
| C4-V3-C12 | KQIINMWQVVGKAMYA-trpSnntrEsirigpGqtfyatg |
| C4-V3-C13 | KQIINMWQVVGKAMYA-trpnnntrksMrigpGqtfyatg |
| C4-V3-C14 | KQIINMWQVVGKAMYA-trpGnntrksMrigpGqtfyatg |
| C4-V3-C15 | KQIINMWQVVGKAMYA-trpGnntrksirigpGqtLyatg |
| C4-V3-C16 | KQIINMWQVVGKAMYA-VrpnnntrksVrigpGqtSyatg |
| C4-V3-C17 | KQIINMWQVVGKAMYA-trpGnntrRsirgpGqtfyatg |
| C4-V3-C18 | KQIINMWQVVGKAMYA-IrpGnntrksVrigpGqtfyatg |
| C4-V3-C19 | KQIINMWQVVGKAMYA-trpnnntrksirigpGqAfyatN |
| C4-V3-C20 | KQIINMWQVVGKAMYA-trpnnntrQsirigpGqAfyatK |
| C4-V3-C21 | KQIINMWQVVGKAMYA-trpGnntrksirigpGqAfFatg |
| C4-V3-C22 | KQIINMWQVVGKAMYA-trpGnntrksVrigpGqAfyatN |
| C4-V3-C23 | KQIINMWQVVGKAMYA-trpnnntrkGiHigpGqAfyaAg |
| C4-V3-C24 | KQIINMWQVVGKAMYA-trpnnntrkGiGigpGqtfFatE |
| C4-V3-C25 | KQIINMWQVVGKAMYA-trpGnntrEsiGigpGqAfyatg |

TABLE 7
C4-V3 peptides Clade B

| | |
|---|---|
| C4-V3-396.2 | KQIINMWQVVGKAMYA-RPNNNTRRNIHIGLGRRFYAT-* |
| C4-V3-170.6 | KQIINMWQVVGKAMYA-RPNNNTRRSVRIGPGGAMFRTG* |
| C4-V3-82.15 | KQIINMWQVVGKAMYA-RPNNNTRRSIPIGPGRAFYTTG* |
| C4-V3-144.8 | KQIINMWQVVGKAMYA-RPDNNTVRKIPIGPGSSFYTT-* |
| C4-V3-23.38 | KQIINMWQVVGKAMYA-RPIKIERKRIPLGLGKAFYTTK* |
| C4-V3-365.2 | KQIINMWQVVGKAMYA-RPSNNTRKGIHLGPGRAIYATE* |
| C4-V3-513.2 | KQIINMWQVVGKAMYA-RPSNNTRKGIHMGPGKAIYTTD* |
| C4-V3-1448.1 | KQLINMWQVVGKAMYA-RPGNTTRRGIPIGPGRAFFTTG* |
| C4-V3-69.18 | KQIINMWQVVGKAMYA-RPNNNTRKSIRIGPGRAVYATD* |
| C4-V3-146.8 | KQIINMWQVVGKAMYA-RPGNNTRRRISIGPGRAFVATK* |
| C4-V3-113.1 | KQIINMWQVVGKAMYA-RPNNNTRRSIHLGMGRALYATG-* |
| C4-V3-51.23 | KQIINMWQVVGKAMYA-RPSNNTRRSIHMGLGRAFYTTG-* |
| C4-V3-72.18 | KQIINMWQVVGYAMYA-RPNNNTRKGINIGPGRAFYATG-* |
| C4-V3-36.29 | KQIINMWQVVGKAMYA-RPNNNTRKGIHIGPGRTFFATG-* |
| C4-V3-70.18 | KQIINMWQVVGKAMYA-RPNNNTRKRIRIGHIGPGRAFYATG* |
| C4-V3-89.14 | KQIINMWQVVGKAMYA-RPSINKRRHIHIGPGRAFYAT-* |
| C4-V3-163.7 | KQIINMWQVVGKAMYA-RLYNYRRKGIHIGPGRAIYATG* |
| C4-V3-57.20 | KQIINMWQVVGKAMYA-RPNRHTGKSIRMGLGRAWHTTR* |
| C4-V3-11.85 | KQIINMWQVVGKAMYA-RPNNNTRKSINIGPGRAFYTTG---* |
| C4-V3-34.29 | KQIINMWQVVGKAMYA-RPNNNTRKSIQIGPGRAFYTTG---* |
| C4-V3-1.481 | KQIINMWQVVGKAMYA-RPNNNTRKSIHIGPGRAFYTTG---* |
| C4-V3-85.15 | KQIINMWQVVGKAMYA-RPNNNTRKSIHIAPGRAFYTTG---* |
| C4-V3-62.19 | KQIINMWQVVGKAMYA-RPNNNTRKSIHIGPGRAFYATE------* |
| C4-V3-125.9 | KQIINMWQVVGKAMYA-RPNNNTRRRISMGPGRVLYTTG* |
| C4-V3-35.29 | KQIINMWQVVGKAMYA-RPNNNTRKRISLGPGRVYYTTG* |
| C4-V3-74.17 | KQIINMWQVVGKAMYA-RPNNNTRKRMTLGPGKVFYTTG* |
| C4-V3-46.26 | KQIINMWQVVGKAMYA-RPDNTIKQRIIHIGPGRPFYTT-* |
| C4-V3-122.9 | KQIINMWQVVGKAMYA-RPNYNETKRIRIHRGYGRSFVTVR* |
| C4-V3-162.7 | KQIINMWQVVGKAMYA-RPGNNTRGSIHLHPGRKFYYSR* |
| C4-V3-3.323 | KQIINMWQVVGKAMYA-RPNNNTRKSINMGPGRAFYTTG |

While the above is offered by way of example, it will be appreciated that the same analyses can by performed for HIV Clades A, D, E, F, G, H, M, N, O, etc, to design V3 immunogens that react with HIV primary isolates from these Clades.

In addition to the sequences described in Tables 6 and 7, a substitution has been made in the C4 sequence at position 9 from E to V to enhance the binding of the C4 region to human immune cell membranes, and to increase immunogenicity (see Example that follows). Substituting V for E at position 9 of C4 results in the C4-E9V-V3RF(A) peptide inducing 2-3 logs higher anti-gp 120 V3 region antibody levels compared with the original C4-V3RFA(A) peptide. The effect of the E9V substitution is not species specific. While not wishing to be bound by theory, the data may indicate that the ability of the E9V variant peptide to enhance B cell antibody production is not MHC specific but rather it relates in some manner to non-MHC specific factors, such as the ability of the peptides to bind to the lipid bilayer of immune cells. The data presented in FIG. 3 demonstrate the ability of C4_{E9V}-V389.6 peptides to bind to human PB lymhocytes and monocytes. The ability of the C4 and C4E9V “T helper” determinants to facilitate immunogenicity of the V3 region may be due to the ability of helical amphipathic structures to interact with lipid bilayers in a non-MHC related manner and promote peptide internalization. The invention encompasses the use of C4 sequences in addition to those described above.

In addition to the composition described above, the invention encompasses each of the hybrid peptides disclosed as well as each of the components (C4 and V3), alone or in covalent or non-covalent association with other sequences, as well as nucleic acid sequences encoding any and all such peptides. The invention provides an HIV immunogen that can induce broadly reactive neutralizing antibodies against HIV of multiple quasispecies, and across clades. With reference to Example 3, the “dual D” HIV isolate, neutralized by serum from GP 469 immunized with peptide 62.19 to a titer of 1:30, is a Clade A/G recombinant HIV isolate. This demonstrates that this peptide (62.19), for example, can induce antibodies against a non-B HIV isolate. The 62.19 and other V3 sequences in FIG. 6 and Tables 10, 11 and 12 can be expressed either alone or, for example. as a C4-V3 sequence, as in FIG. 6. It will be appreciated that the same analysis described in Example 3 can by performed for any of HIV Clades A, D, E, F, G, H, M, N, O, etc, to identify V3 immunogens that react with HIV primary isolates from one or more of these Clades.

The peptide immunogens of the invention can be chemically synthesized and purified using methods which are well known to the ordinarily skilled artisan. (See, for example, the Example that follows.) The composition can comprise the peptides linked end to end or can comprise a mixture of individual peptides. The peptide immunogens can also be synthesized by well-known recombinant DNA techniques. Recombinant synthesis may be preferred when the peptides are covalently linked. Nucleic acids encoding the peptides of the invention can be used as components of, for example, a DNA vaccine wherein the peptide encoding sequence(s) is/are administered as naked DNA or, for example, a minigene encoding the peptides can be present in a viral vector. The encoding sequence(s) can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki Forest Virus vector, or a Tobacco Mosaic Virus vector. The encoding sequence(s), can also be expressed as a DNA plasmid with, for example, an active promoter such as a CMV promoter. Other live vectors can also be used to express the sequences of the invention. Expression of the immunogenic peptides of the invention can be induced in a patient's own cells, by introduction into those cells of nucleic acids that encode the peptides, preferably using codons and promoters that optimize expression in human cells. Examples of methods of making and using DNA vaccines are disclosed in U.S. Pat. Nos. 5,580,859, 5,589,466, and 5,703,055.

The composition of the invention comprises an immunologically effective amount of the peptide immunogens of this invention, or nucleic acid sequence(s) encoding same, in a pharmaceutically acceptable delivery system. The compositions can be used for prevention and/or treatment of immunodeficiency virus infection. The compositions of the invention can be formulated using adjuvants, emulsifiers, pharmaceutically-acceptable carriers or other ingredients routinely provided in vaccine compositions. Optimum formulations can be readily designed by one of ordinary skill in the art and can include formulations for immediate release and/or for sustained release, and for induction of systemic immunity and/or induction of localized mucosal immunity (e.g, the formulation can be designed for intranasal administration). The present compositions can be administered by any convenient route including subcutaneous, intranasal, oral, intramuscular, or other parenteral or enteral route. The immunogens can be administered as a single dose or multiple doses. Optimum immunization schedules can be readily determined by the ordinarily skilled artisan and can vary with the patient, the composition and the effect sought. By way of example, it is noted that approximately 50 μg-100 μg of each hybrid peptide can be administered, for example, intramuscularly (e.g. 3×).

The invention contemplates the direct use of both the peptides of the invention and/or nucleic acids encoding same and/or the peptides expressed as minigenes in the vectors indicated above. For example, a minigene encoding the peptides can be used as a prime and/or boost. Importantly, it has been recently shown that recombinant gp120 is not efficacious as a vaccine for HIV in phase III trials (Elias, P., Durham Morning Herald, Feb. 25, 2003; VaxGen News Conference, Feb. 24, 2003). Thus, it would be advantageous to express, for example, the 62.19 V3 loop and/or other V3 loops in Table 11 or 12 in the context of gp120 molecules or gp160 or gp140 molecules, either as expressed soluble recombinant proteins, or expressed in the context of one of the vectors described above. This strategy takes advantage of the ability to express native V3 conformations within a whole gp120 or gp140 or gp160 HIV envelope protein.

One of the preferred gp120, gp140 or gp160 envelopes that, for example, 62.19 V3 loops can be expressed with is that of consensus or ancestral HIV envelope artificial sequences (Gaaschen et al, Science 296:2354-2360 (2002)). Although artificial and computer designed, one such sequence (the consensus of consensus envelope) gp120 (con 6) has been shown to bind soluble CD4 and anti-gp120 mabs A32, 1b12, 2G12. After binding mab A32 or soluble CD4, the con 6 gp120 binds the CCR5 binding site mab 176—indicating a “native” gp120 conformation.

Thus, the entire V3 loops from the Los Alamos Database from the sequences of one or more of the peptides in Table 11 or 12 can be expressed in the consensus (con 6) or other consensus or ancestral gp120, gp140, or gp160 envelope protein, or expressed in a native gp120, gp140, or gp160, such as HIV BAL or HIV JRFL, and used as an immunogen as a recombinant envelope protein, or used as an immunogen expressed in one of the vectors above.

The V3 peptides or recombinant proteins can be used as primes or boosts with the V3 peptides or recombinant gp120s, gp140s or gp160s expressed in the above vectors used as primes or boosts.

A preferred immunogen is the consensus 6 gp120 expressing the full-length 62.19 V3 loop, expressed as a DNA plasmid as a primary immunization, followed by adenovirus expressing the Con 6 envelope expressing the 62.19 V3 sequence from the Los Alamos Database as a booster immunization.

In addition to the polypeptides described above and in the Examples that follow, the invention further relates to modified forms thereof (and nucleic acid sequences encoding same) wherein negatively charged amino acids are added/substituted on the right hand side of the loop and positively charged amino acids are added/substituted on the left hand side. Such additions/substitutions can serve to stabilize the “hairpin” as a result of hydrogen bonding between the oppositely charged amino acids. Conversely, negatively charged amino acids can be added/substituted on the left hand side of the loop and positively charged on the right. Examples of such sequences are as follows (in the “B” and “C” type polypeptides shown below, it will be appreciated that the D and the E can be reversed as well at the ends of the peptides—further, for example, DDD can be present at the night hand side of the loop, as can EEE):

TABLE 5

| | |
|---|---|
| For 62.19 | |
| | * |
| 62.19A | C4-TRPNNNTRKSIHIGPGRAFYATED |
| 62.19B | C4-TRPNNNTRKSIHIGPGRAFYATEDE |
| 62.19C | C4-TRPNNNRRKSIHIGPGRAFYATEDE |
| 62.19D | TRPNNNTRKSIHIGPGRAFYATED |
| 62.19E | TRPNNNTRKSIHIGPGRAFYATEDE |
| 62.19F | TRPNNNRRKSIHIGPGRAFYATEDE |
| 62.19G | C4-TRPNNNRRKSIHIGPGRAFYATEEE |
| 62.19H | TRPNNNRRKSIHIGPGRAFYATEEE |
| For 1.481 | |
| 1.481A | C4-RPNNNTRKSIHIGPGRAFYTTGD |
| 1.481B | C4-RPNNNTRKSIHIGPGRAFYTTGDE |
| 1.481C | C4-RPNNNRRKSIHIGPGRAFYTTGDE |
| 1.481D | C4-RPNNNRRKSIHIGPGRAFYTTGEE |
| 1.481E | RPNNNTRKSIHIGPGRAFYTTGD |
| 1.481F | RPNNNTRKSIHIGPGRAFYTTGDE |
| 1.481G | RPNNNRRKSIHIGPGRAFYTTGDE |
| 1.481H | RPNNNRRKSIHIGPGRAFYTTGEE |
| For 11.85 | |
| 11.85A | C4-RPNNNTRKSINIGPGRAFYTTGD |
| 11.85B | C4-RPNNNTRKSINIGPGRAFYTTGDE |
| 11.85C | C4-RPNNRTRKSINIGPGRAFYTTGDE |
| 11.85D | C4-RPNNRTRKSINIGPGRAFYTTGEE |
| 11.85E | RPNNNTRKSINIGPGRAFYTTGD |
| 11.85F | RPNNNTRKSINIGPGRAFYTTGDE |
| 11.85G | RPNNRTRKSINIGPGRAFYTTGDE |
| 11.85H | RPNNRTRKSINIGPGRAFYTTGEE |

For 513.2 (RPSNNTRKGIHMGPGKAIYTTD)

| | |
|---|---|
| 513.2A | C4-RPSNNTRKGIHMGPGKAIYTTDD |
| 513.2B | C4-RPSNNTRKGIHMGPGKAIYTTDDE |
| 513.2C | C4-RPSNNRRKGIHMGPGKAIYTTDE |
| 513.2D | C4-RPSNNNRRKGIHMGPGKAIYTTDEE |
| 513.2E | RPSNNTRKGIHMGPGKAIYTTDD |
| 513.2F | RPSNNTRKGIHMGPGKAIYTTDDE |
| 513.2G | RPSNNRRKGIHMGPGKAIYTTDE |
| 513.2H | RPSNNNRRKGIHMGPGKAIYTTDEE |
| For 74.17 | |
| 74.17A | C4-RPNNNTRKRMTLGPGKVFYTTGD |
| 74.17B | C4-RPNNNTRKRMTLGPGKVFYTTGDE |
| 74.17C | C4-RPNNNRRKRMTLGPGKVFYTTGDE |
| 74.17D | C4-RPNNNRRKRMTLGPGKVFYTTGEE |
| 74.17E | RPNNNTRKRMTLGPGKVFYTTGD |
| 74.17F | RPNNNTRKRMTLGPGKVFYTTGDE |
| 74.17G | RPNNNRRKRMTLGPGKVFYTTGDE |
| 74.17H | RPNNNRRKRMTLGPGKVFYTTGEE |
| For 36.29 | |
| 36.29A | C4-RPNNNTRKGIHIGPGRTFFATGD |
| 36.29B | C4-RPNNNTRKGIHIGPGRTFFATG DE |
| 36.29C | C4-RPNNNRRKGIHIGPGRTFFATG DE |
| 36.29B | C4-RPNNNRRKGIHIGPGRTFFATG EE |
| 36.29E | RPNNNTRKGIHIGPGRTFFATG D |
| 36.29F | RPNNNTRKGIHIGPGRTFFATG DE |
| 36.29G | RPNNNRRKGIHIGPGRTFFATG DE |
| 36.29H | RPNNNRRKGIHIGPGRTFFATG EE |
| For 34.29 | |
| 34.29A | C4-RPNNNTRKSIQIGPGRAFYTTG D |
| 34.29B | C4-RPNNNTRKSIQIGPGRAFYTTG DE |
| 34.29C | C4-RPNNNRRKSIQIGPGRAFYTTG DE |
| 34.29D | C4-RPNNNTRRKSIQIGPGRAFYTTG EE |
| 34.29E | RPNNNTRKSIQIGPGRAFYTTG D |
| 34.29F | RPNNNTRKSIQIGPGRAFYTTG DE |
| 34.29G | RPNNNRRKSIQIGPGRAFYTTG DE |
| 34.29H | RPNNNTRRKSIQIGPGRAFYTTG EE |

In the context of the modified forms of 62.19 depicted above, the invention further includes peptides wherein in the R in . . . GPGR . . . (designated with an asterisk) can be substituted with any one of Q, G, K, S, A, L or H.

It will be appreciated from a reading of this disclosure that the foregoing, like others described herein, can be expressed, for example, in gp120, gp140 and that the vectors described above are equally applicable here.

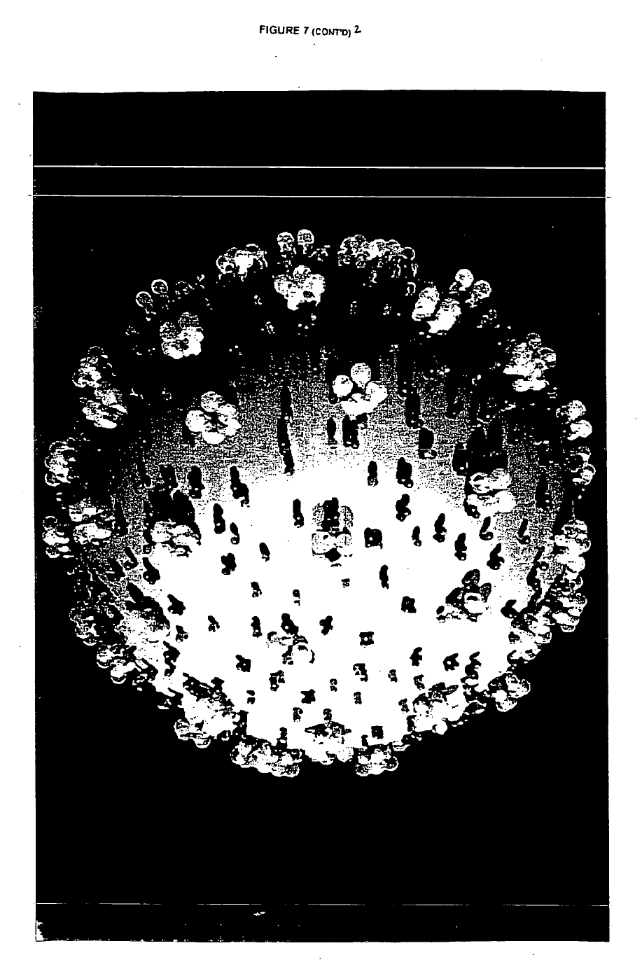

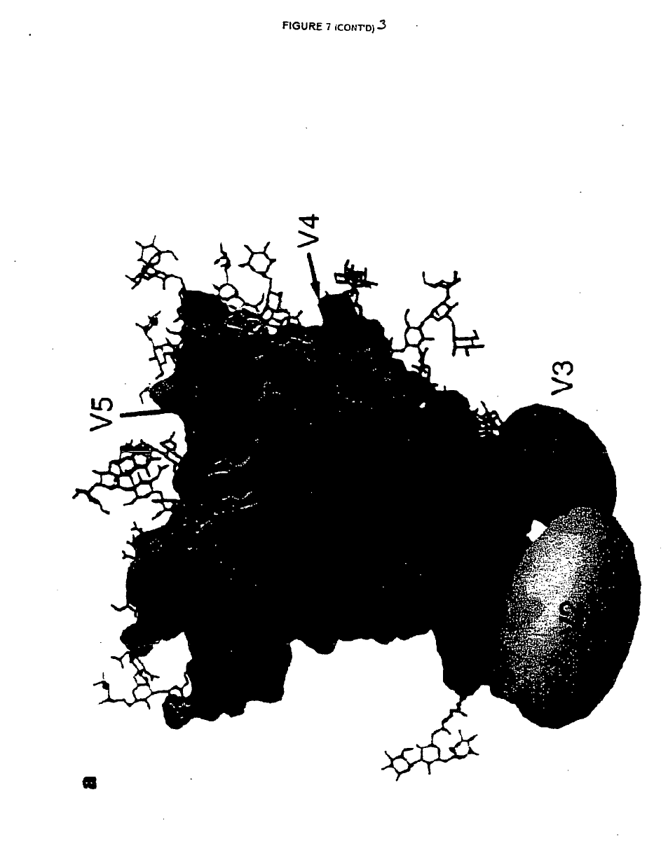

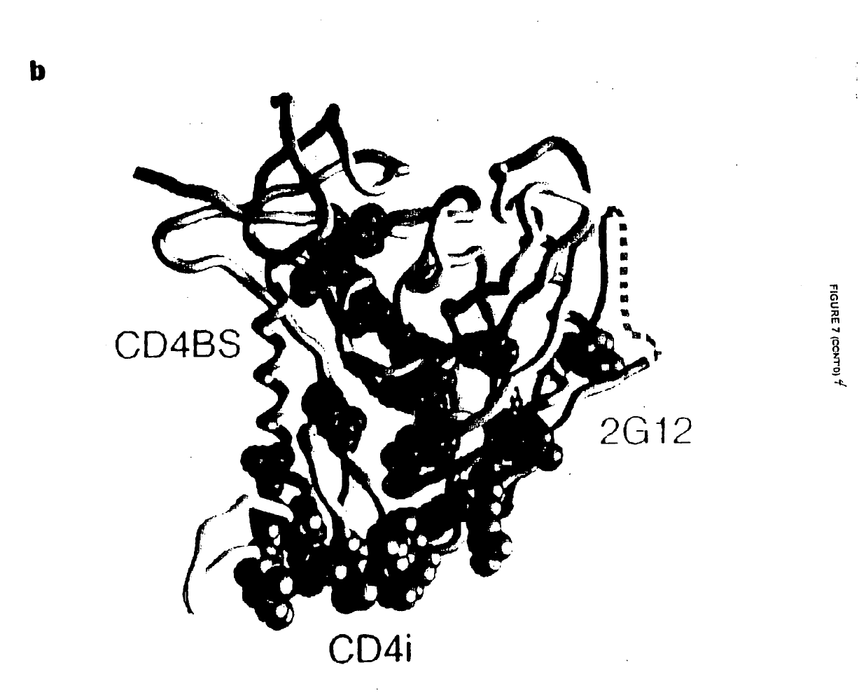

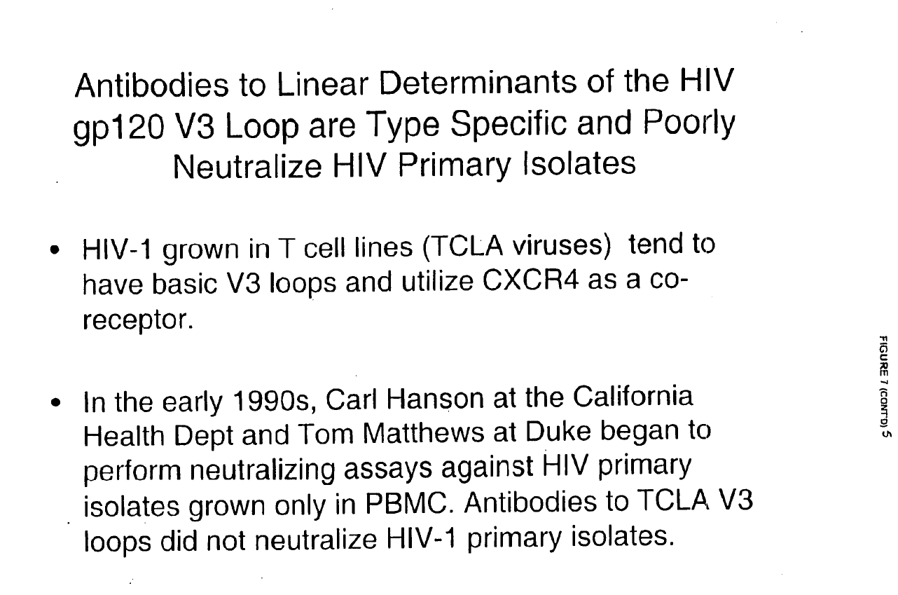

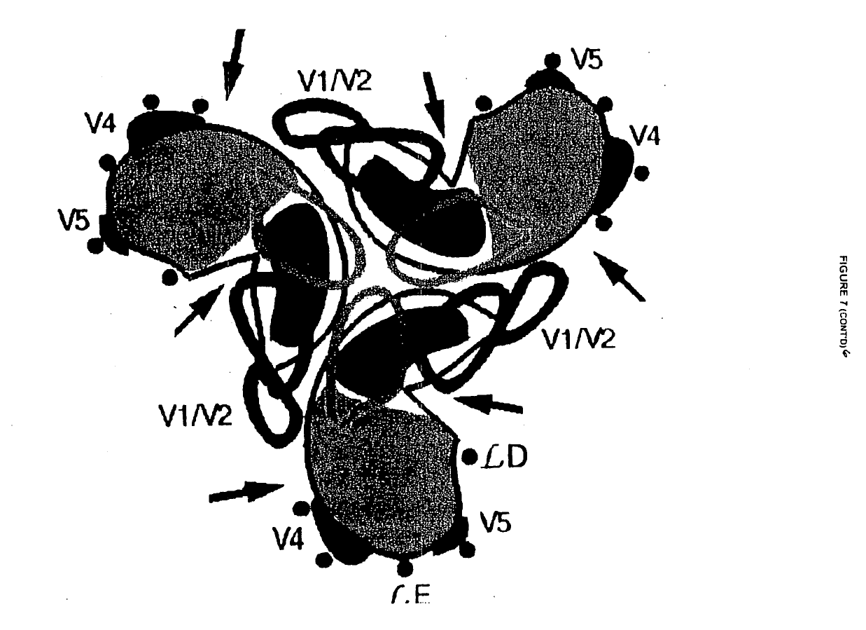

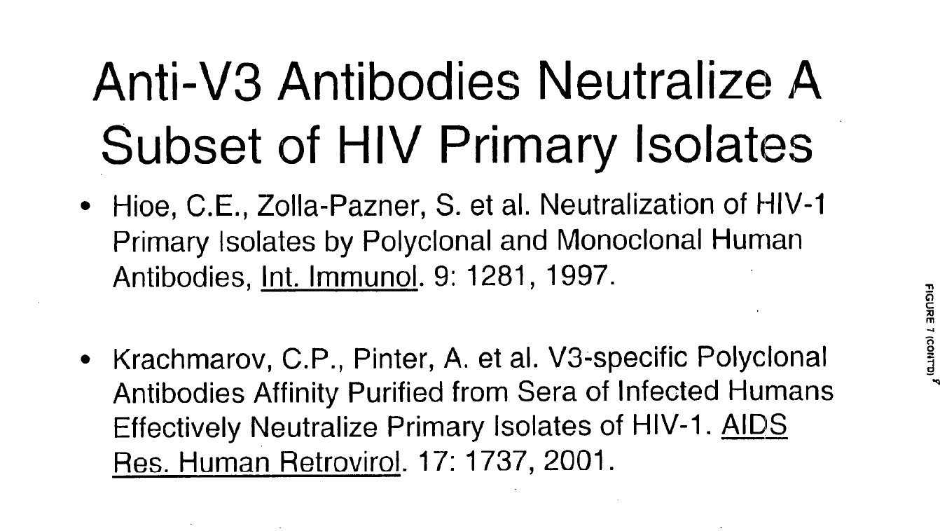

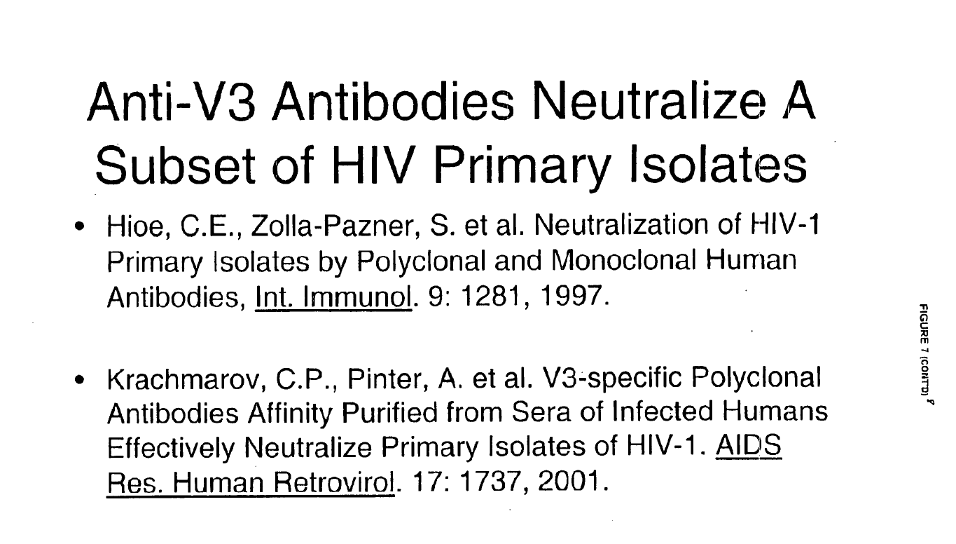

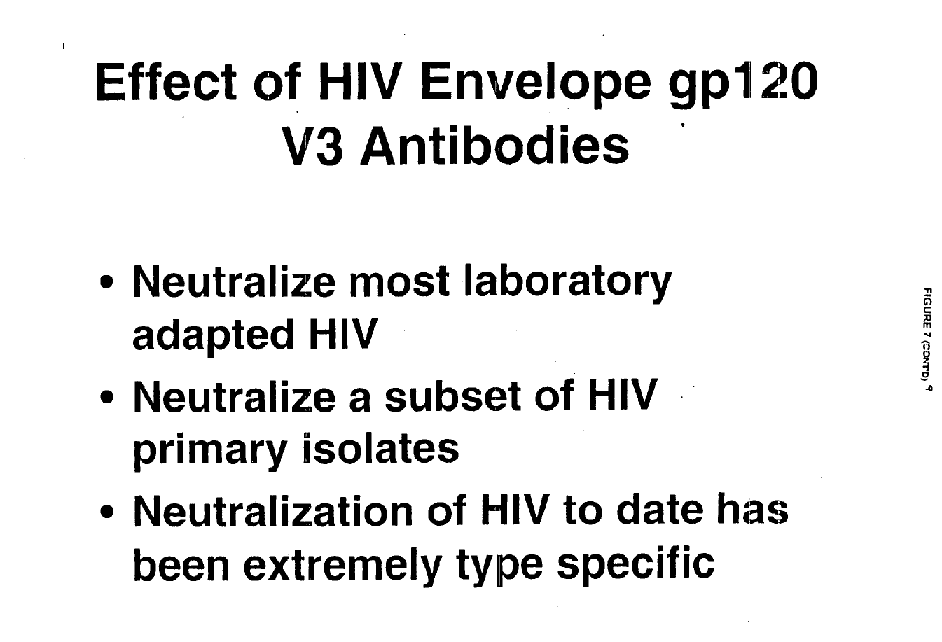

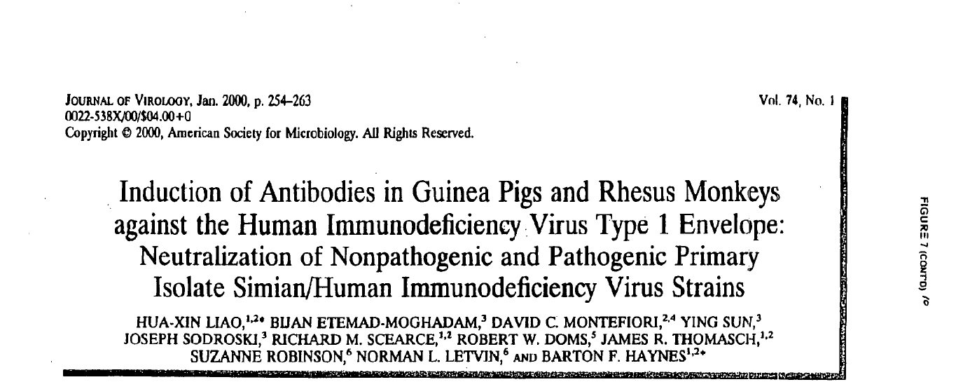

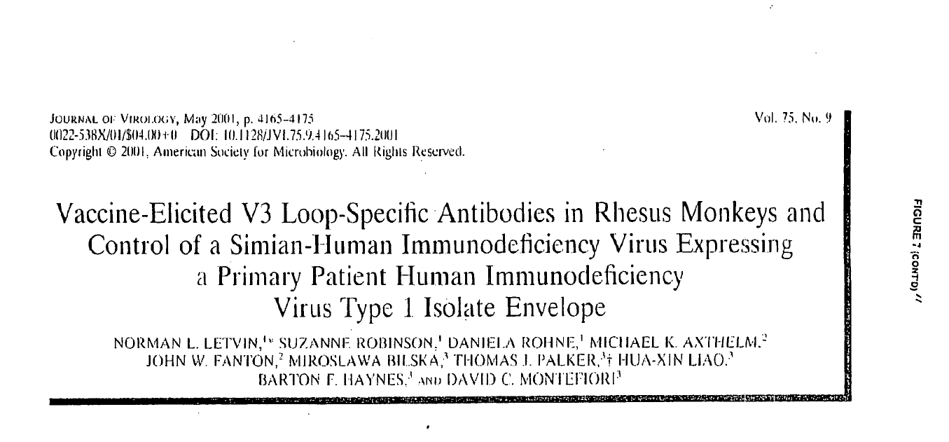

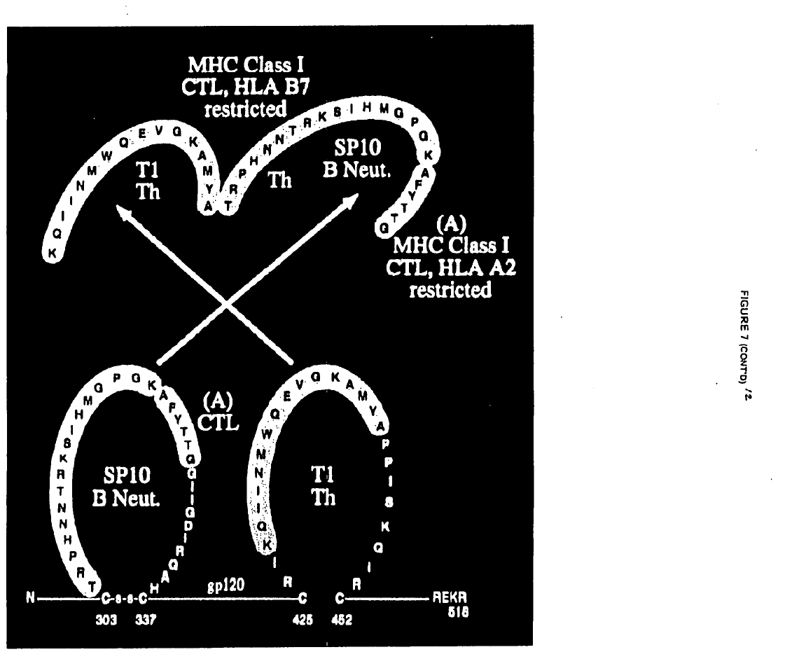

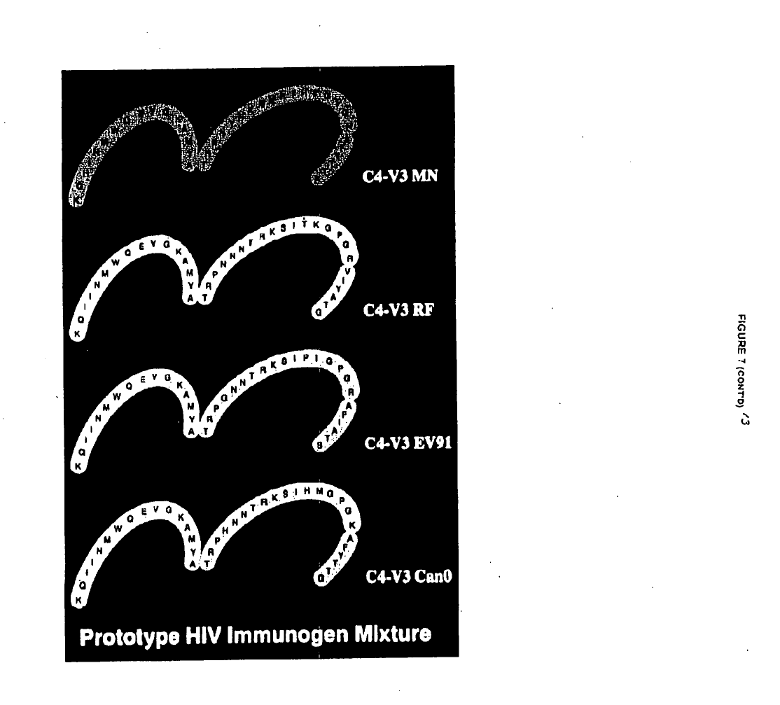

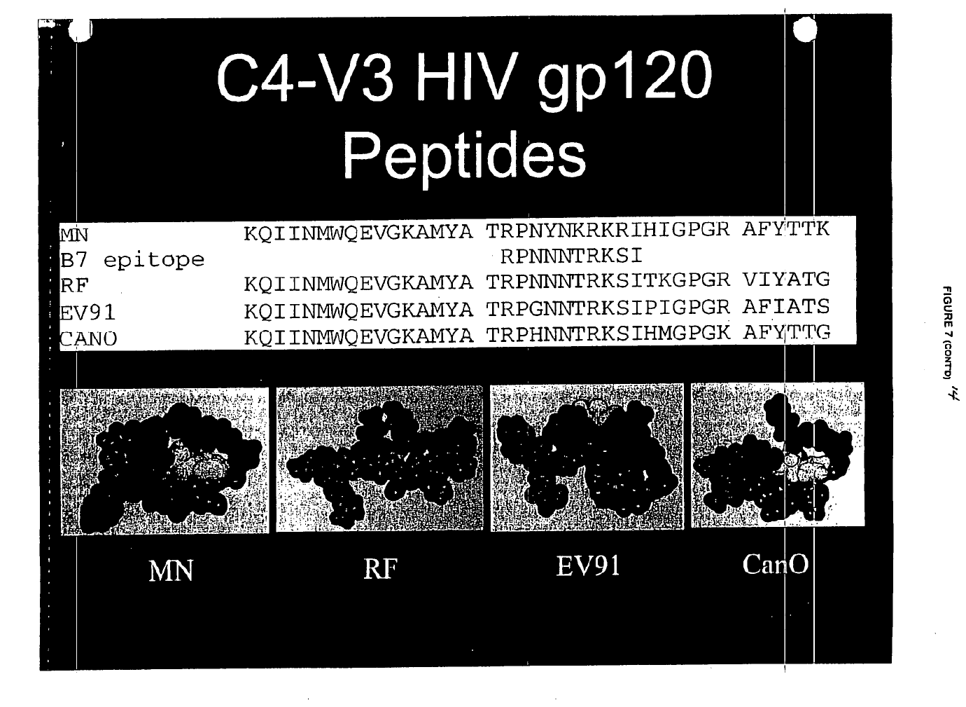

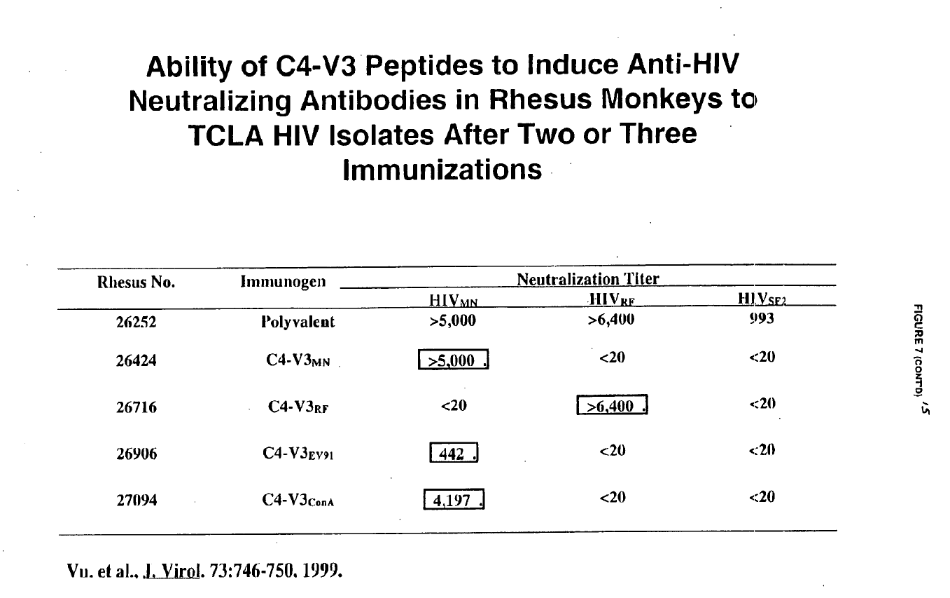

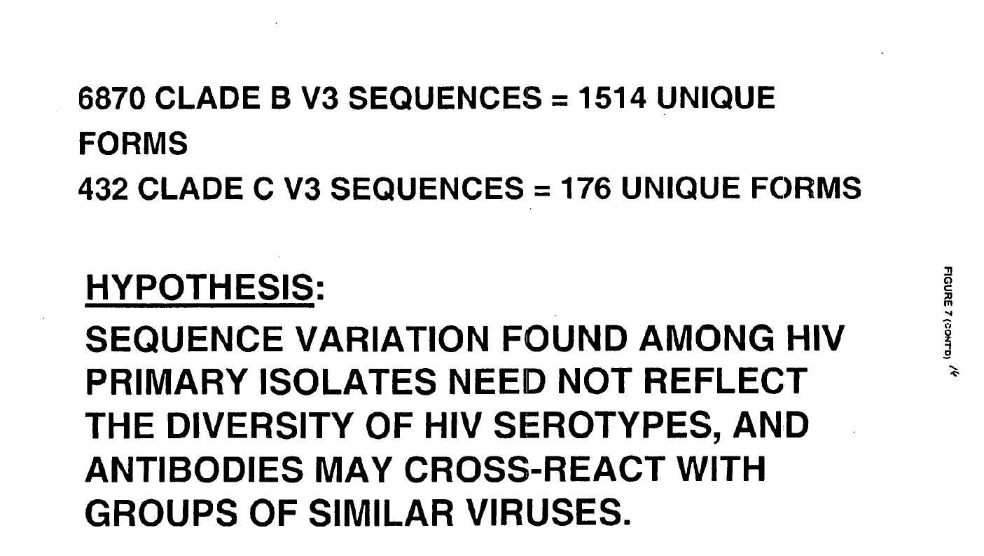

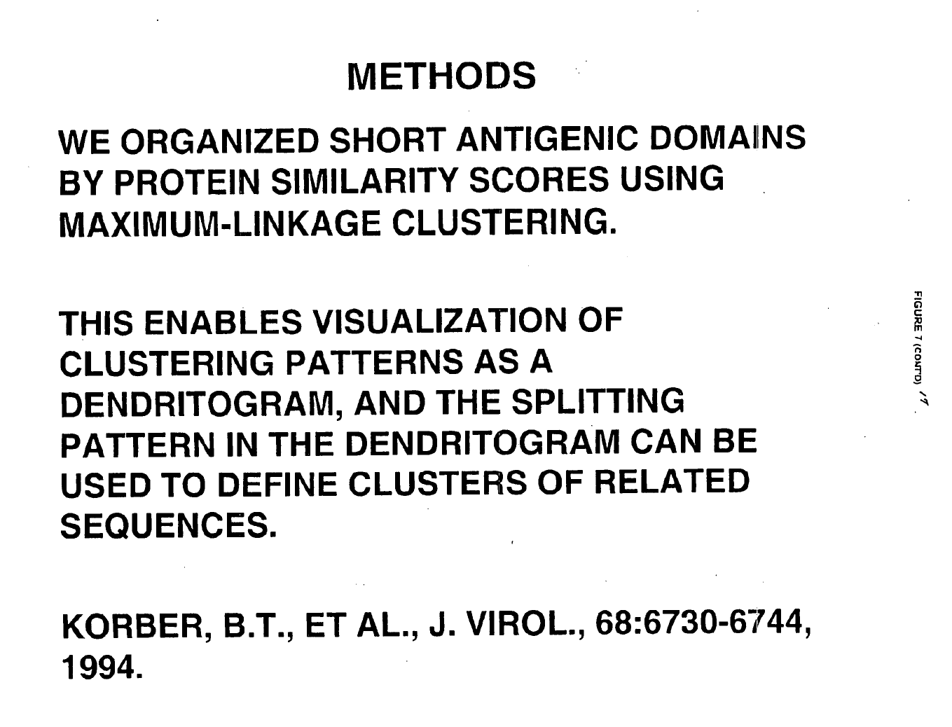

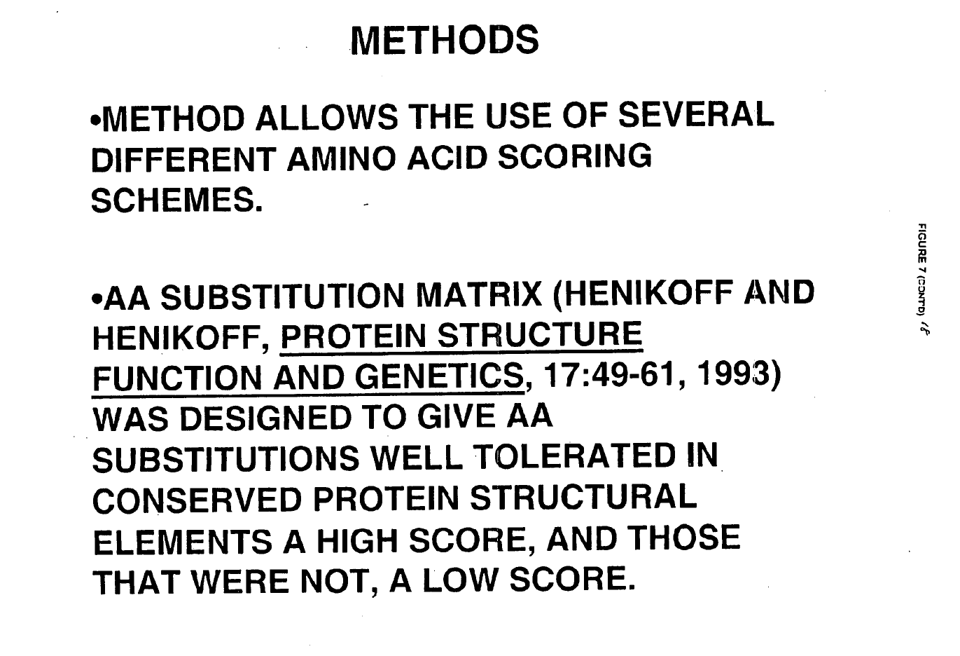

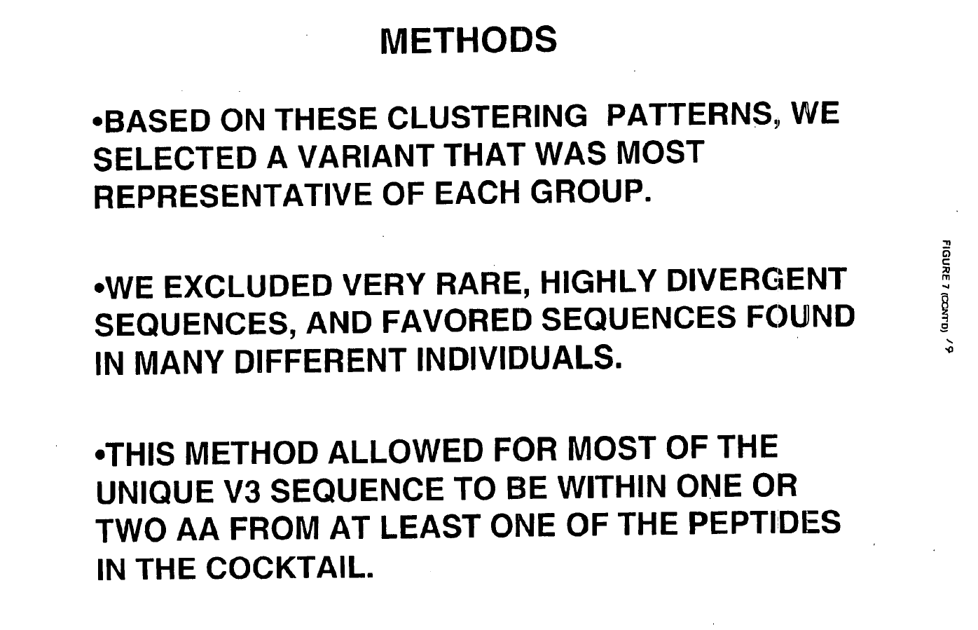

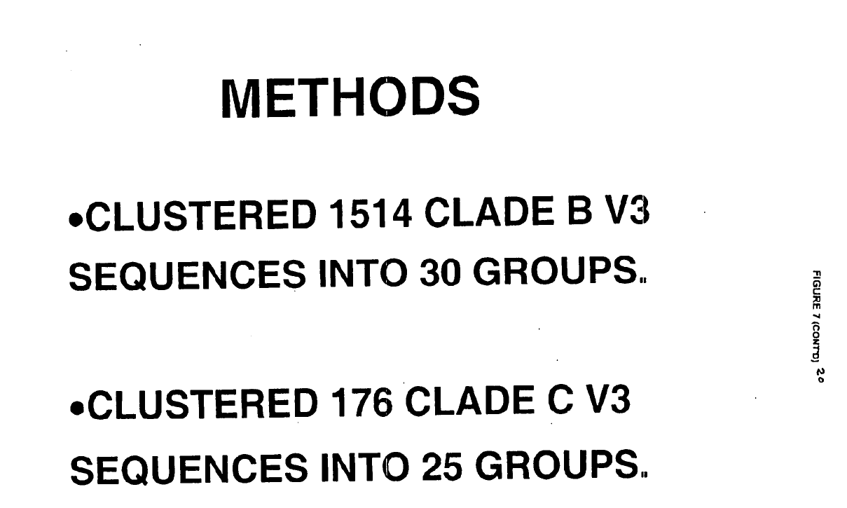

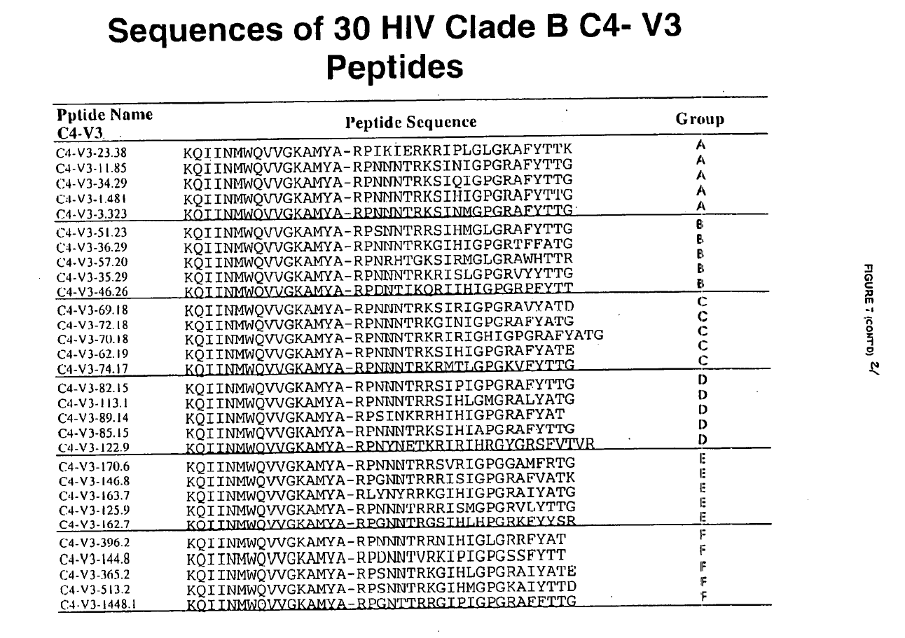

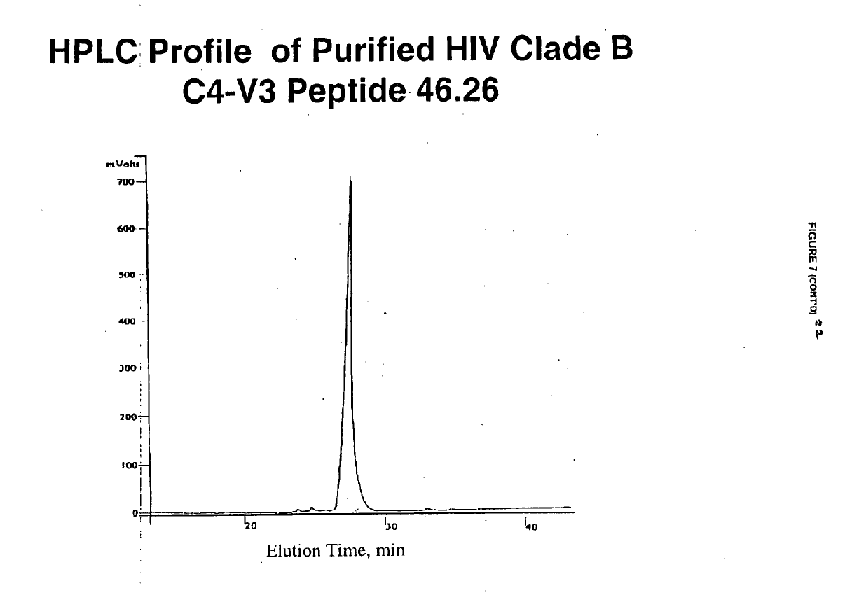

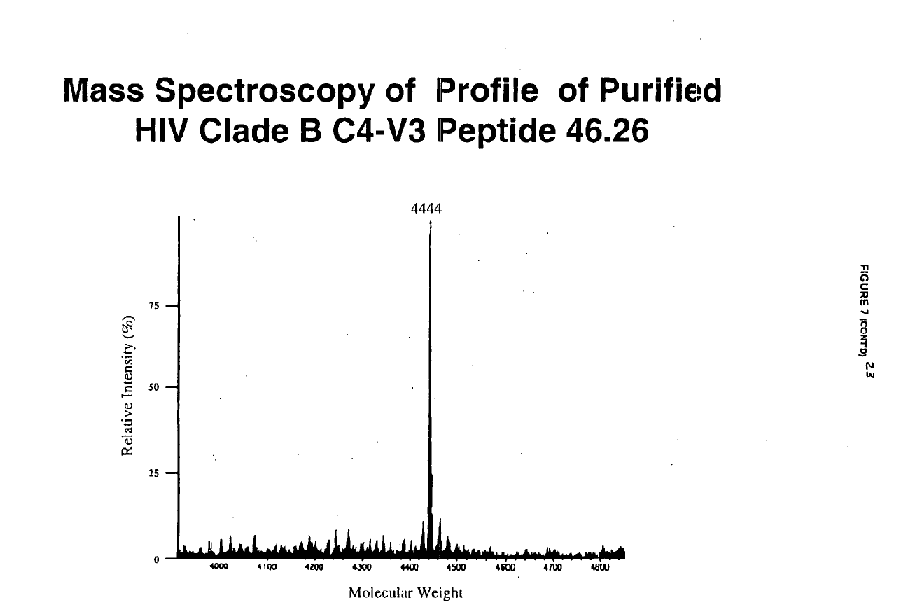

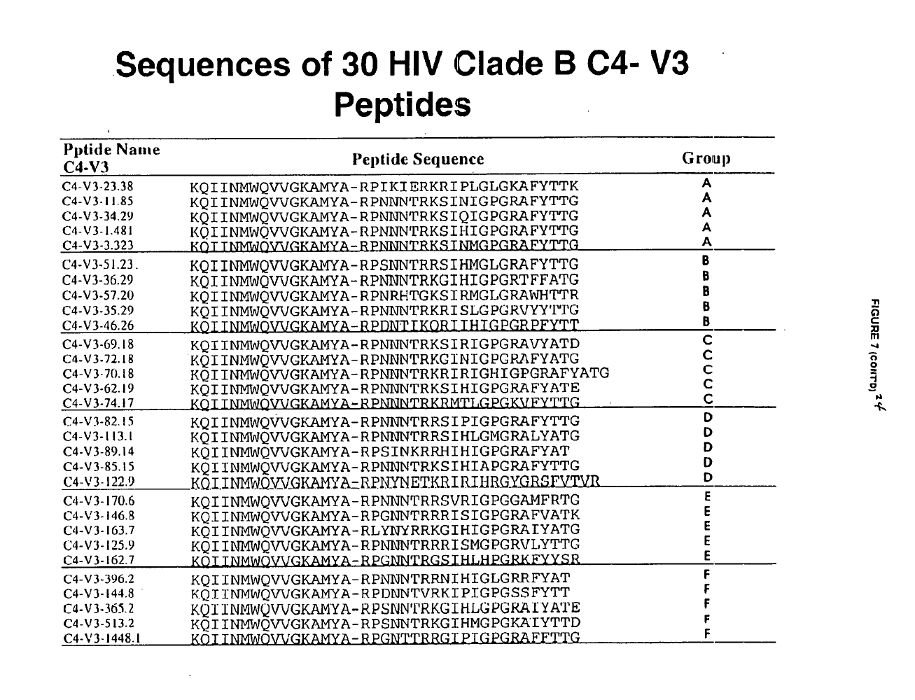

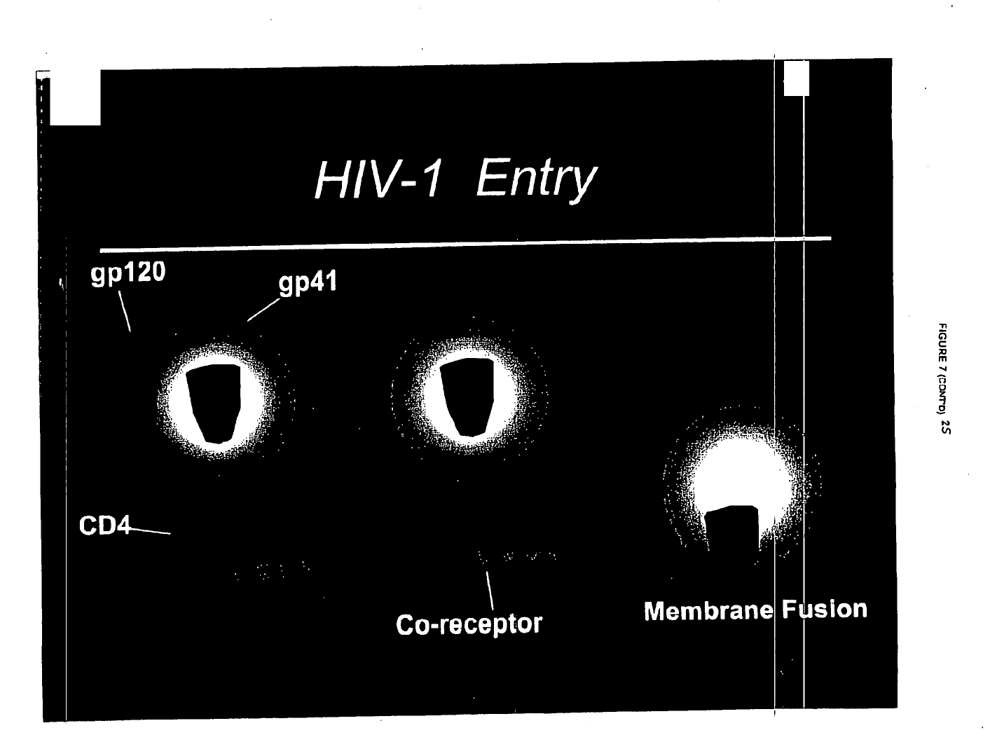

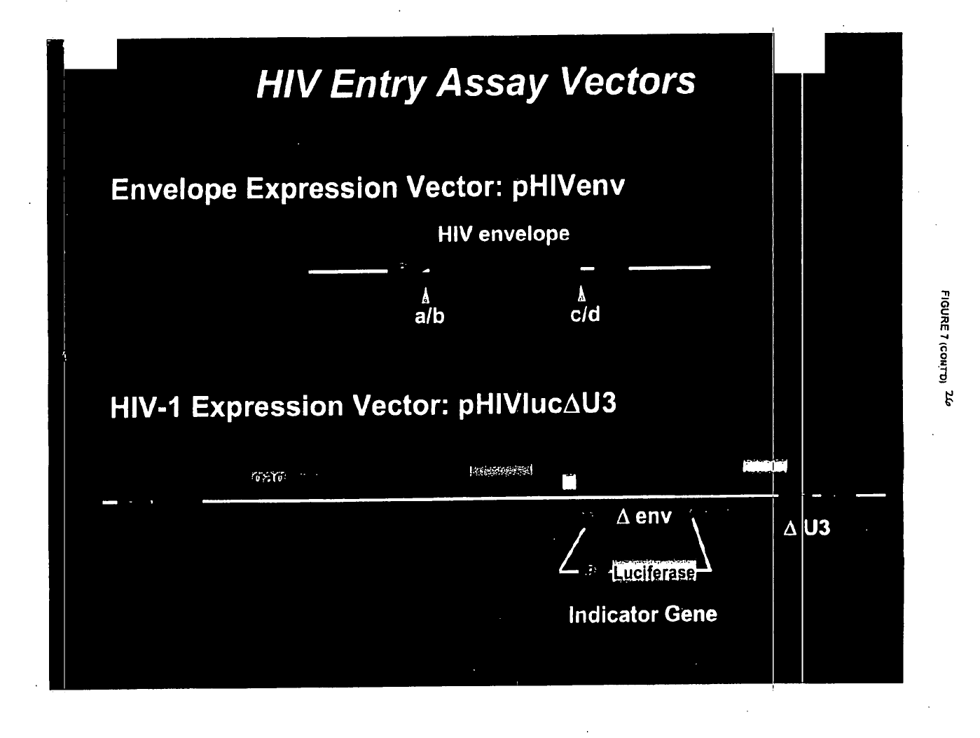

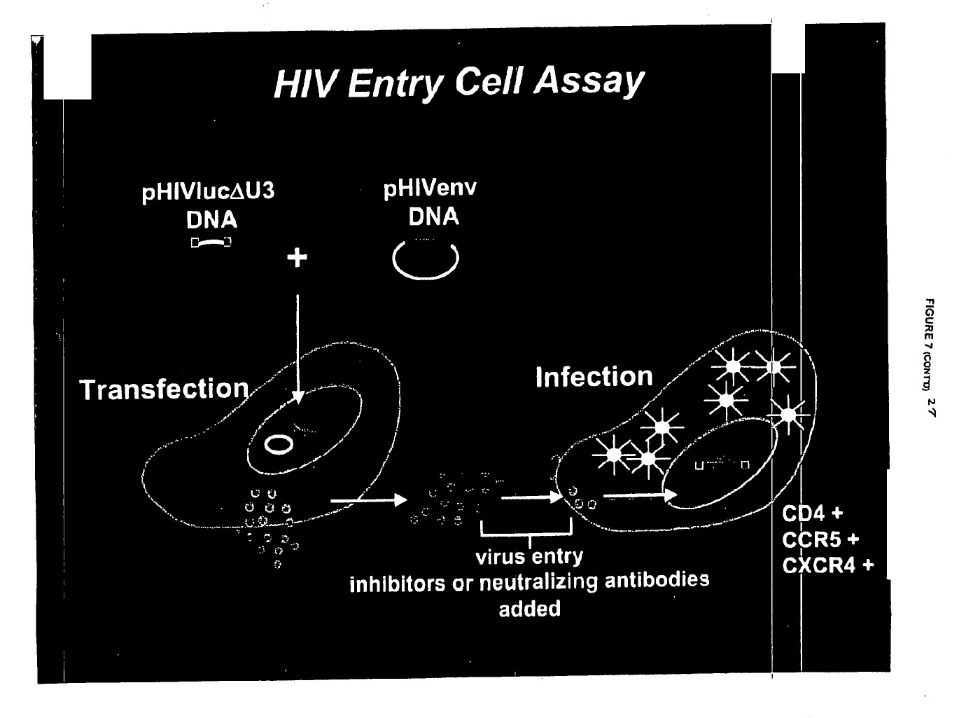

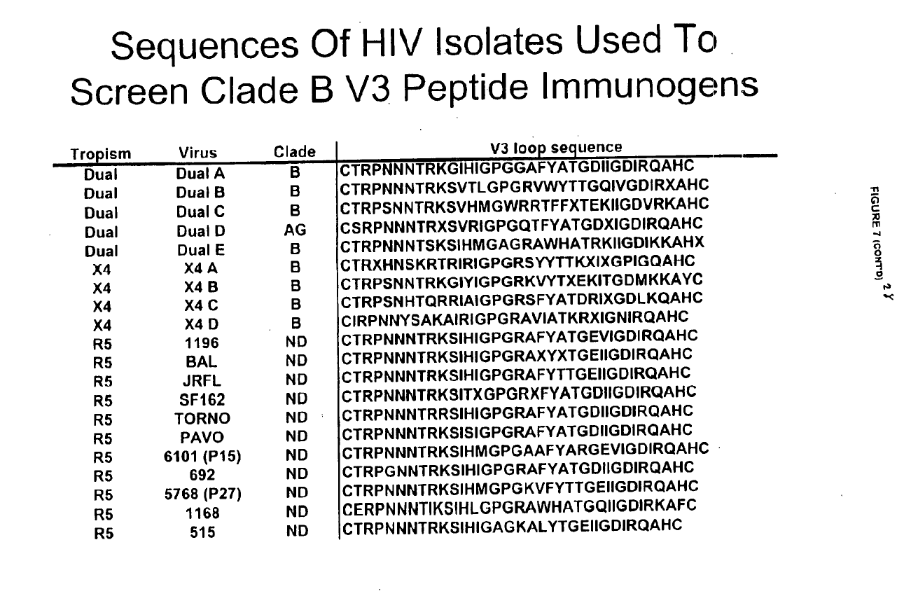

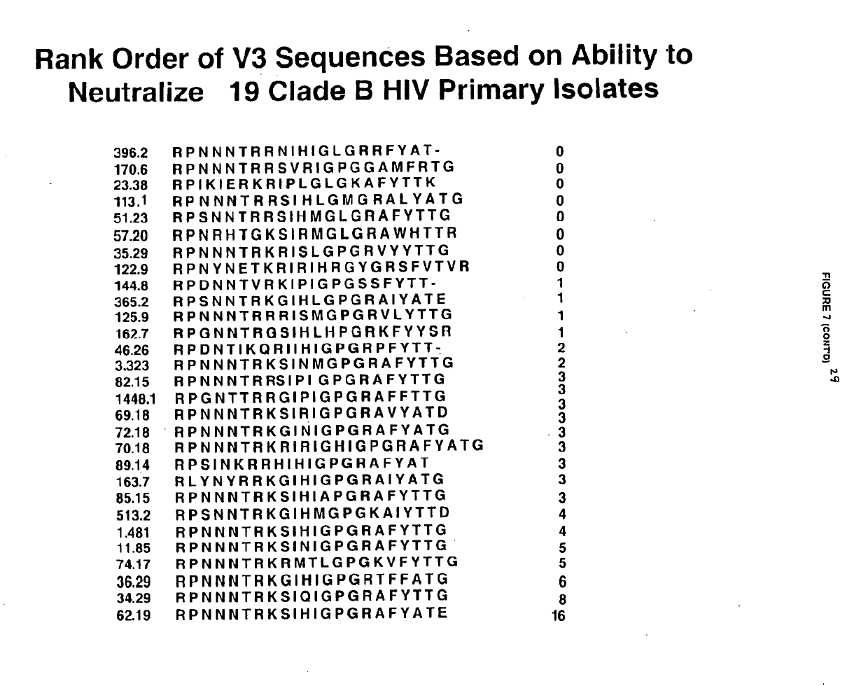

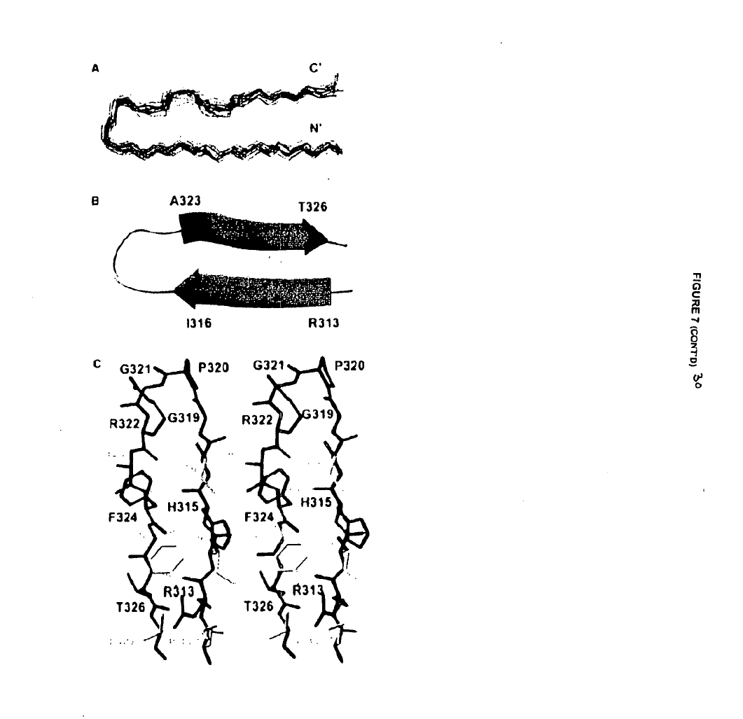

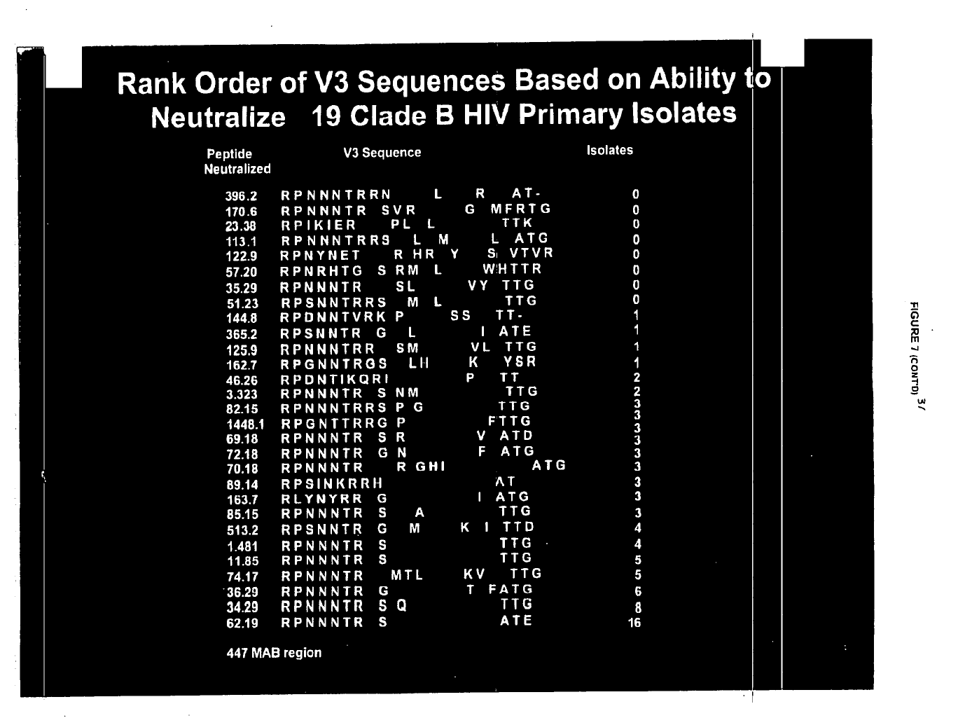

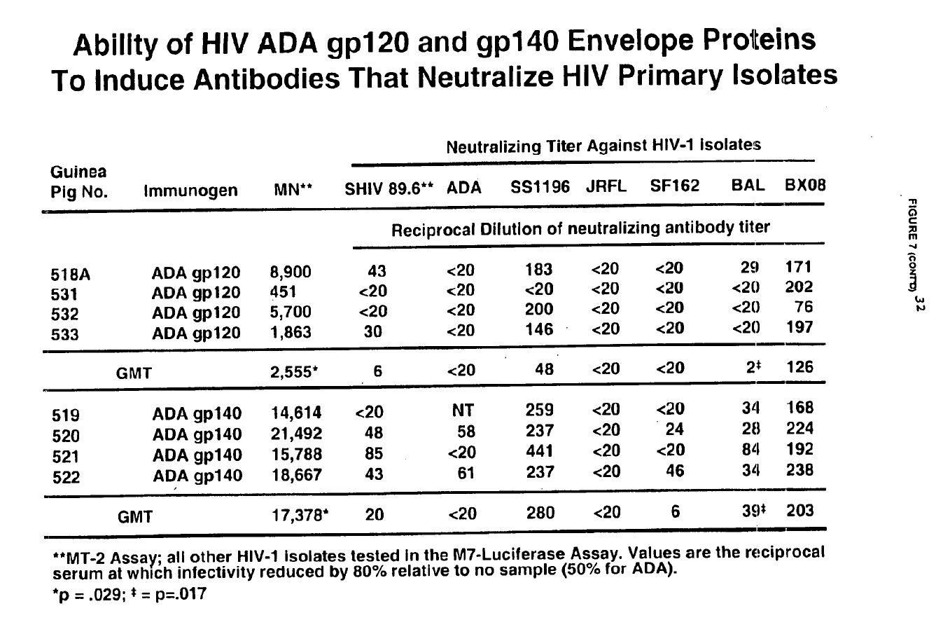

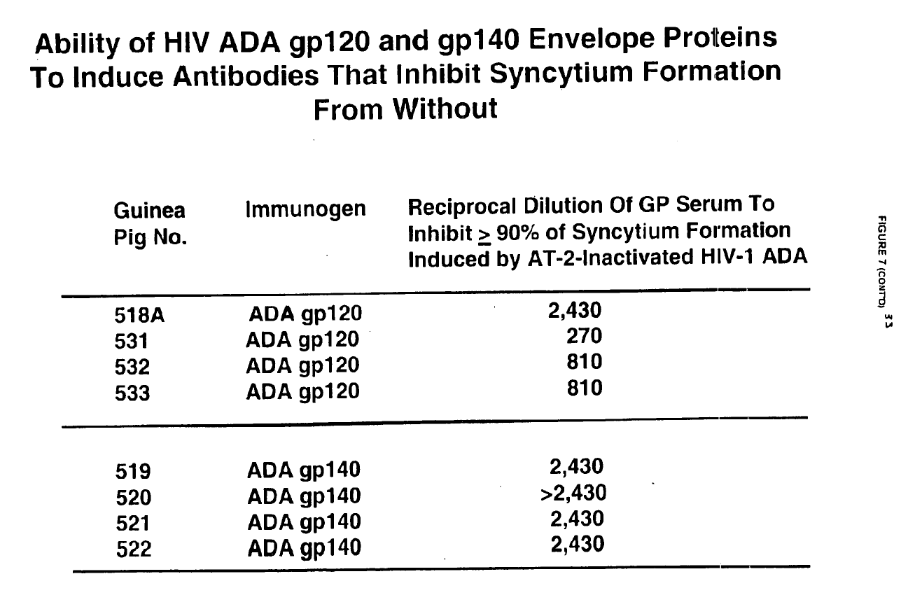

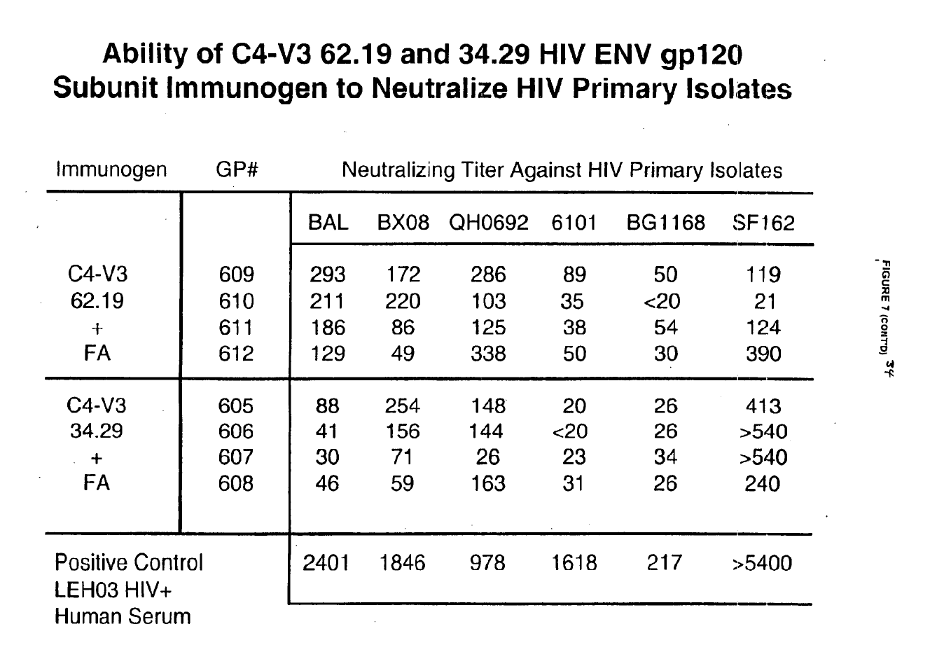

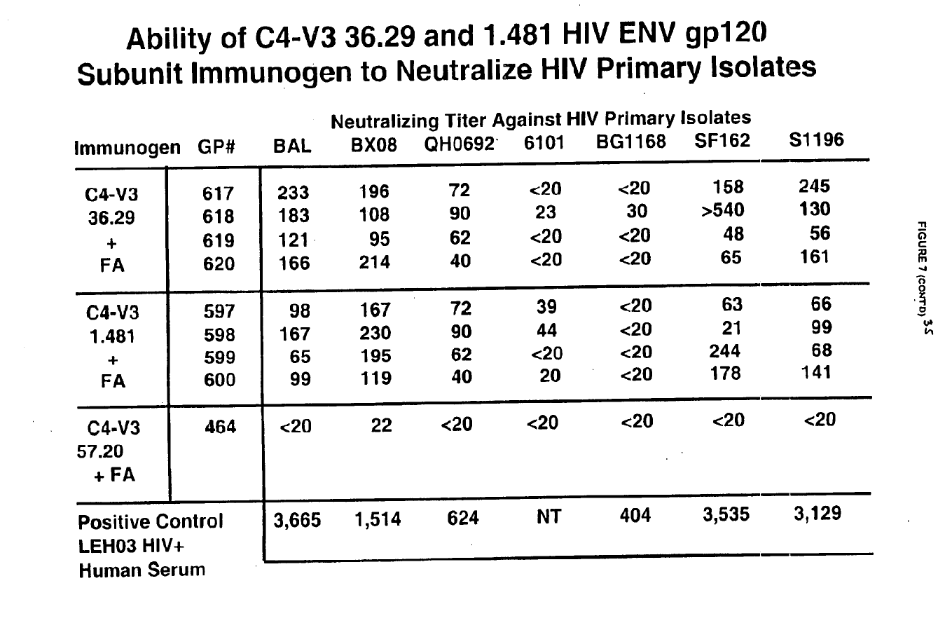

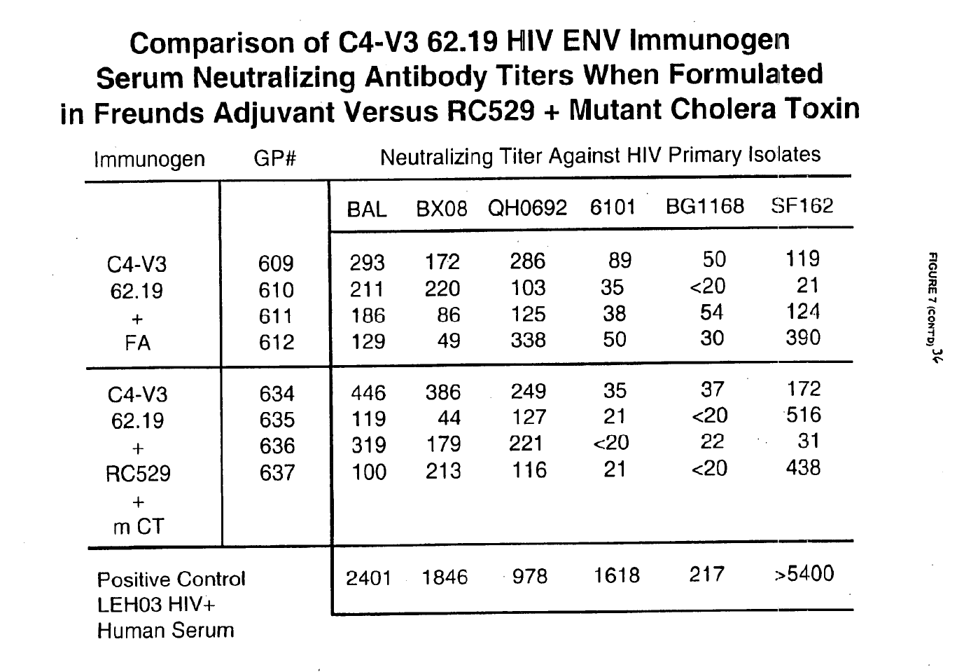

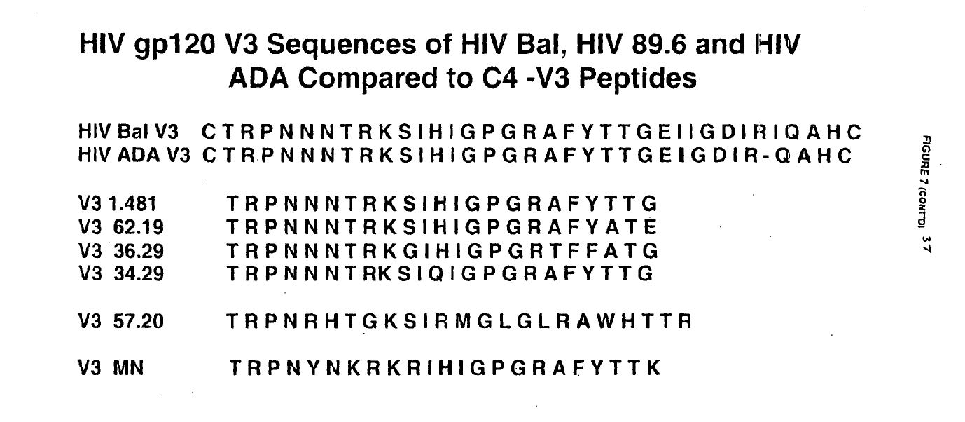

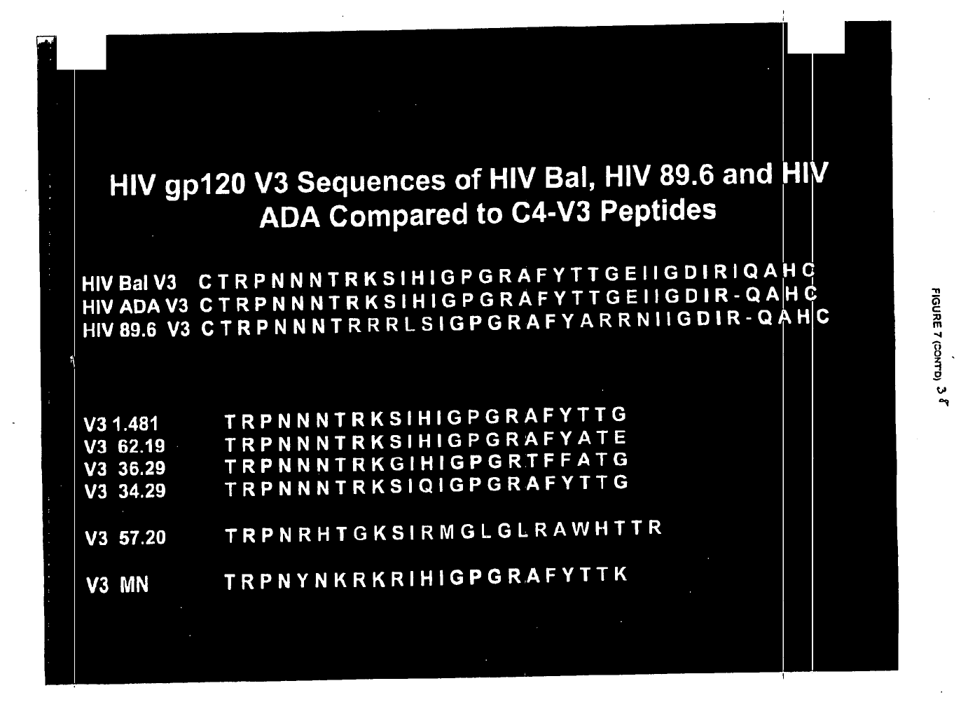

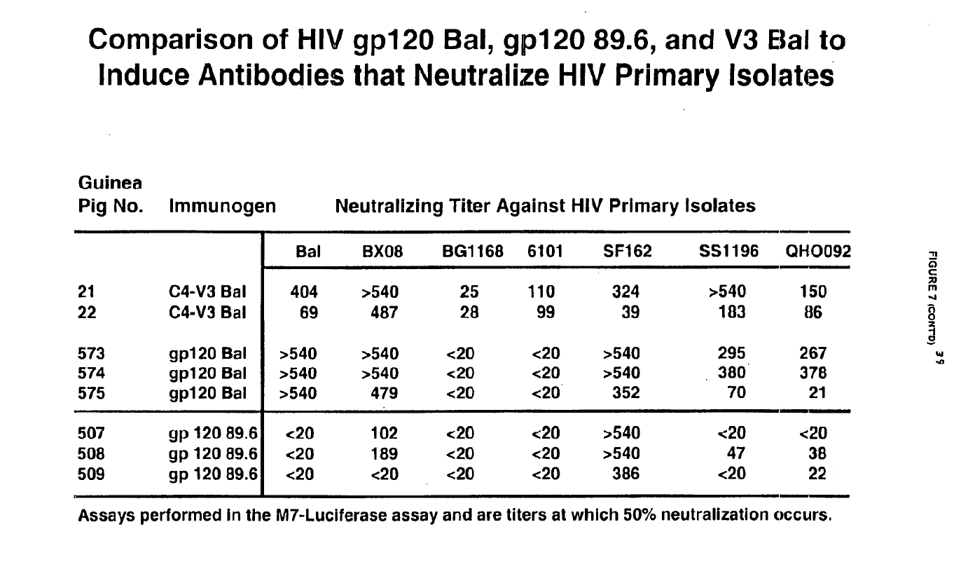

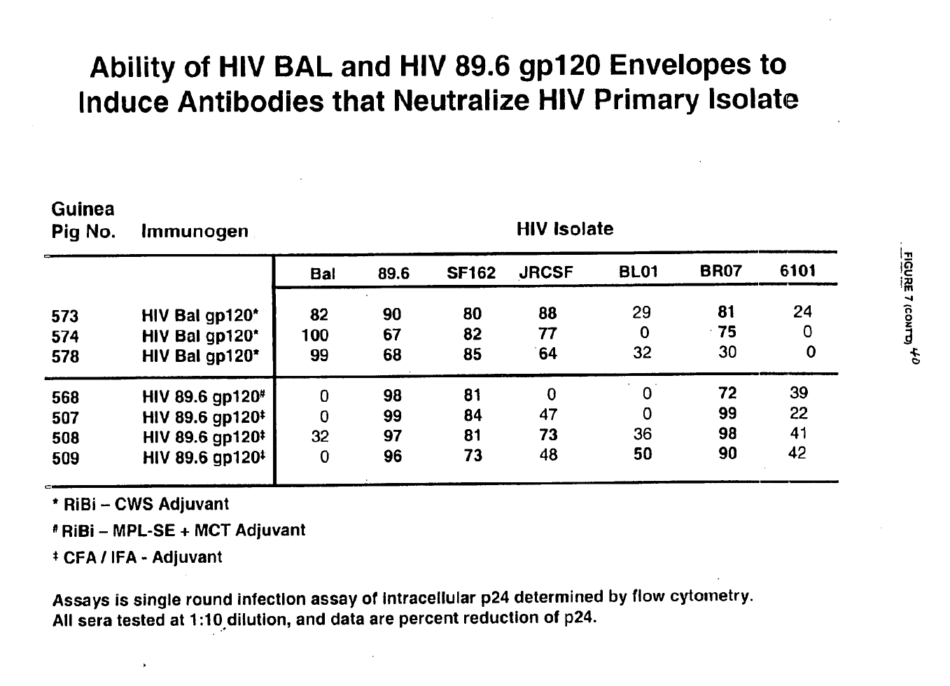

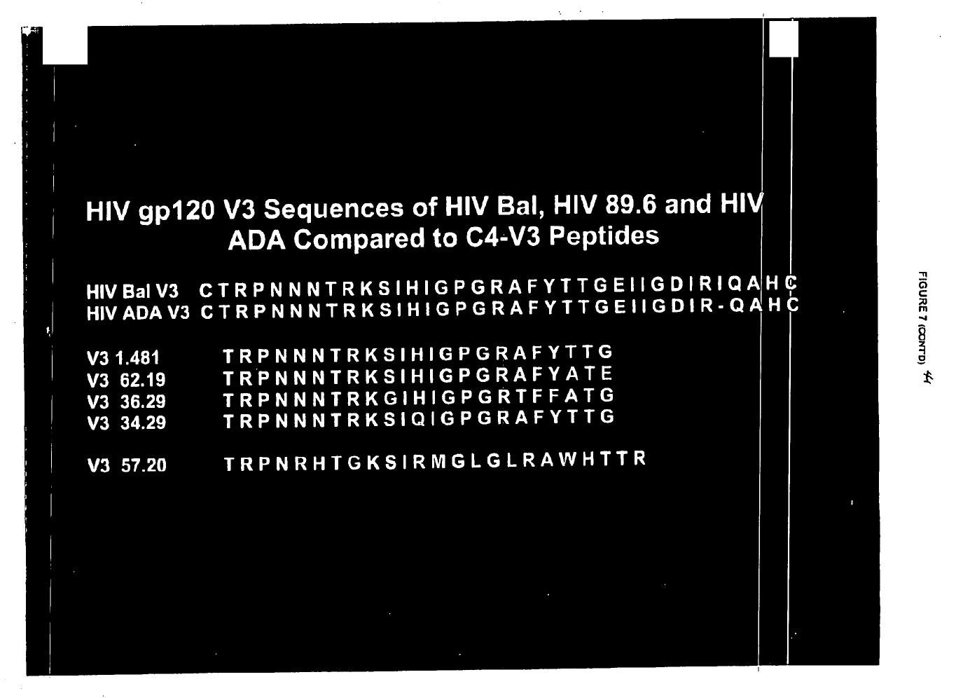

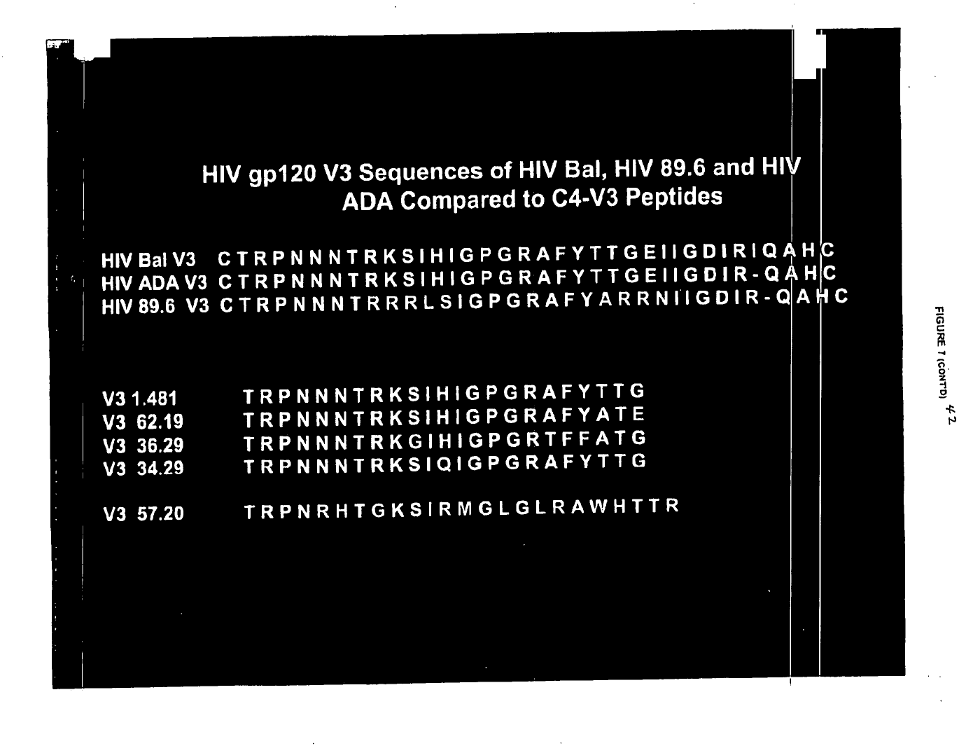

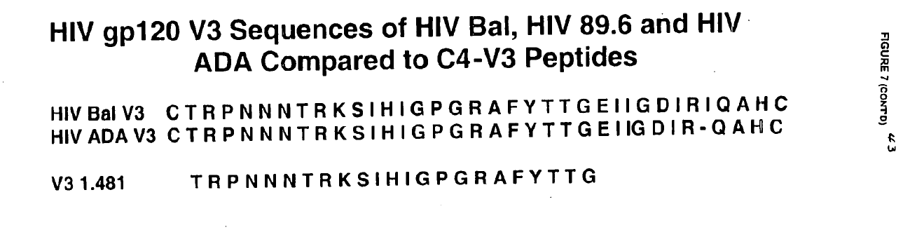

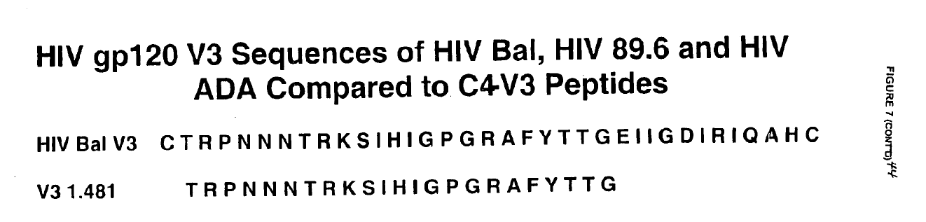

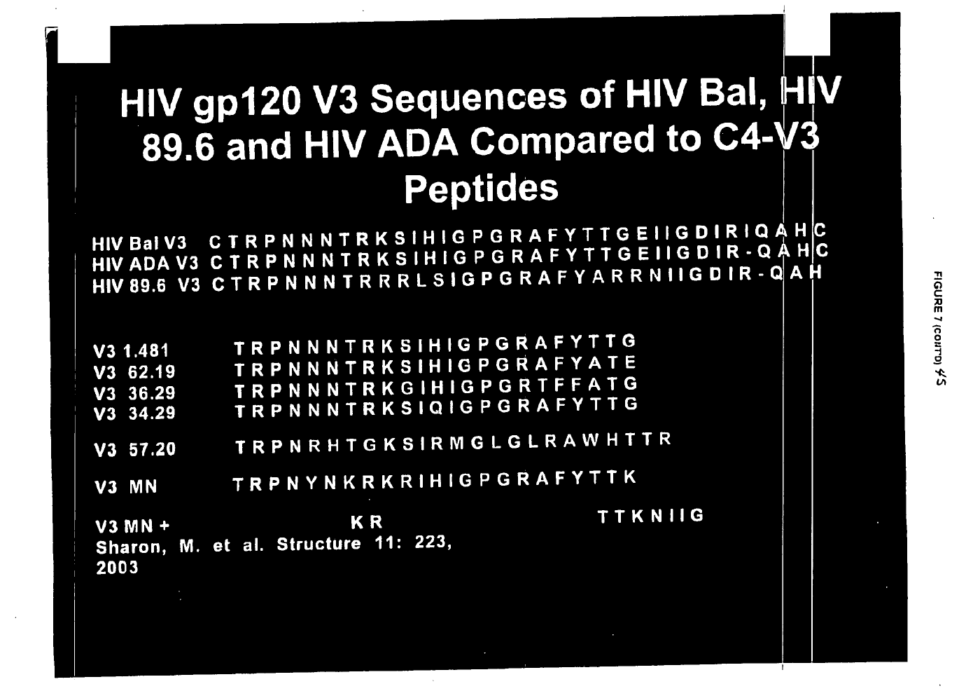

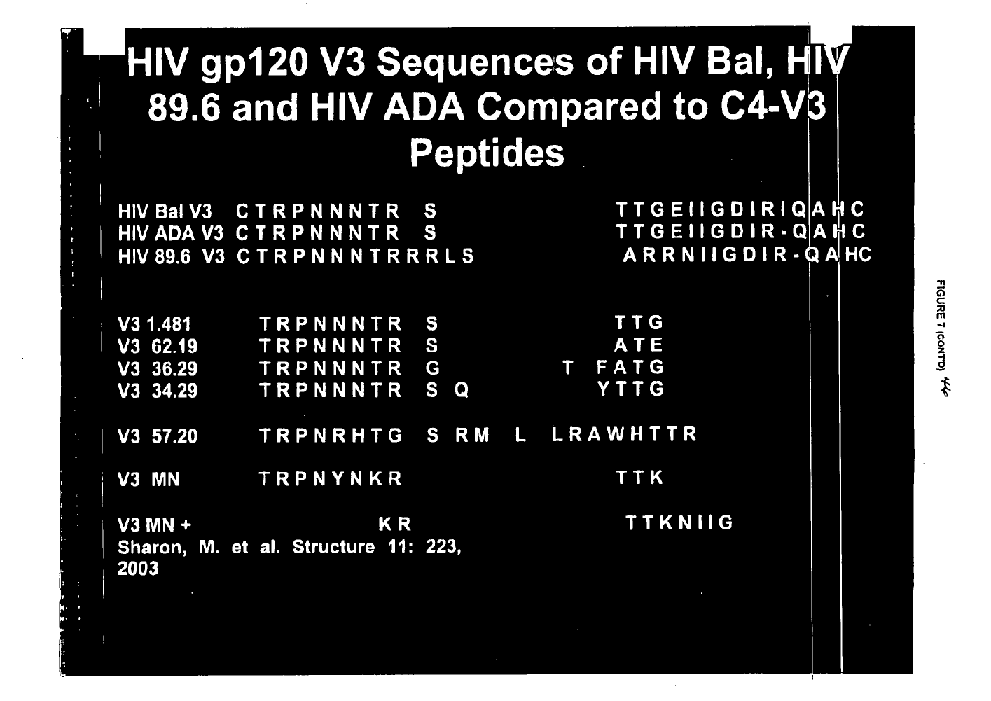

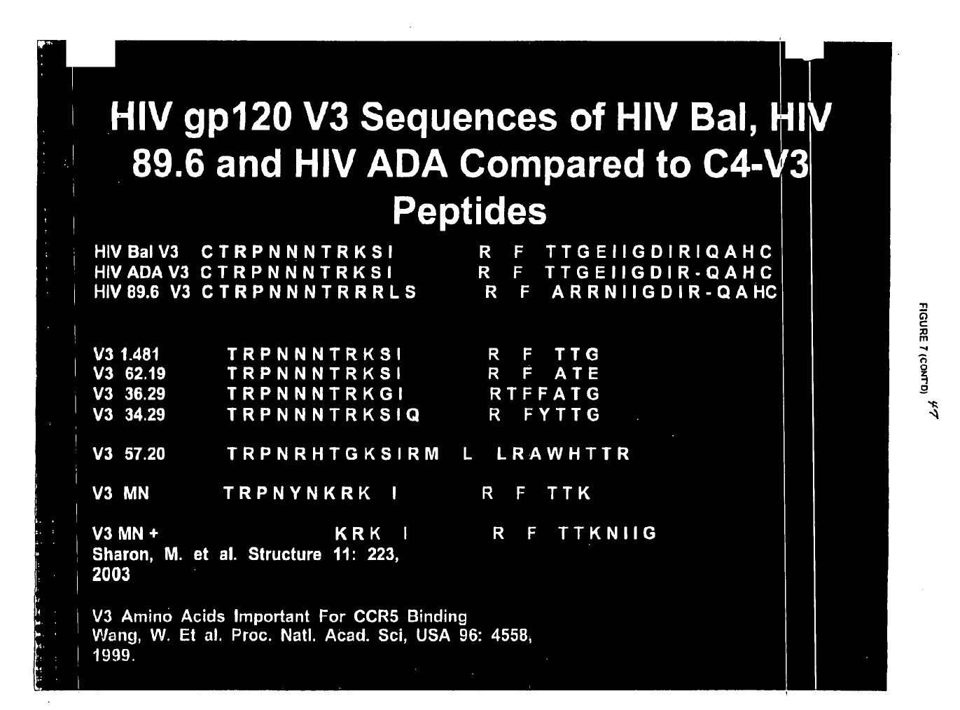

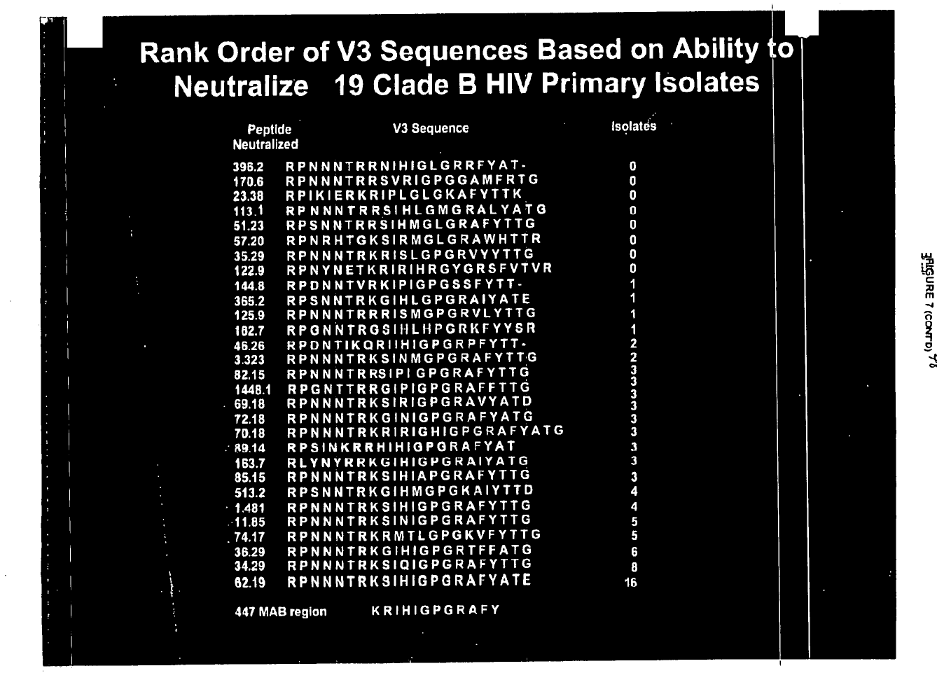

Certain aspects of the invention can be described in greater detail in non-limiting Example that follows. (See also FIG. 7.)

### EXAMPLE 1

### Experimental Details

Peptide Design, Synthesis and Purification.

Peptides were designed, as shown in Table 1. It was hypothesized that alteration of the C4 sequence to reduce its helical conformational tendency in peptides might cause enrichment of solution conformers resembling a D strand conformation. This in turn might cause C4 to be immunogenic for antibodies recognizing the native conformation of the C4 (part of the CD4 binding site) region of gp120. The present work describes tests of this hypothesis in chimeric peptide C4-V3 RF, which has a V3 segment from gp120 of HIV strain RF, and three sequence variants wherein single amino-acid replacements have been introduced at position 9 in the C4 segment, Glu (E) to Gly (G), Glu (E) to Val (V), and at position 12, Lys (K) to Glu (E) (Table 1). These replacements were made in part to disrupt possible stabilization of helical conformations due to side-chain (i, i+3) charge interaction between E9 and K12 (Scholtz et al, Biochemistry 32:9668-9676 (1993)). In addition, the substitution in C4_{E9G}-V3RF(A) was expected to disfavor helix formation by introducing greater main-chain flexibility (Chakrabartty et al, Adv. Protein Chem. 46:141-176 (1995)). Furthermore the substitution in C4_{E9G}-V3RF(A) introduced two adjacent valine residues which has been hypothesized to favor extended conformations. Thus, the parent peptide, C4-V3RF(A) (Haynes et at, AD Res. Human Retroviruses 11:211-221 (1995)) contained 16 N-terminal residues from the C4 domain of gp120_{IIIB} and 23 C-terminal residues from the V3 domain of gp120 of HIVRF.

TABLE 1
Peptides Used in This Study

| Peptide | Sequence |
|---|---|
| | C4 V3 |
| | 1 16 17 39 |

| | |
|---|---|
| C4-V3RF | KQIINMWQEVGKAMYA TRPNNNTRKSITKGPGRVIYATG |
| (A) | |
| C4_{E9G}- | KQIINMWQGVGKAMYA TRPNNNTRKSITKGPGRVIYATG |
| V3RF(A) | |
| C4_{E9V}- | KQI]NMWQVVGKAMYA TRPNNNTRKSITKGPGRVIYATG |
| V3RF(A) | |
| C4_{K12E}- | KQIINMWQEVGEAMYA TRPNNNTRKSITKGPGRVIYATG |
| V3RF(A) | |

All sequences from Los Alamos National Laboratory AIDS Sequence Database.

Peptides were synthesized by fluorenylmethoxycarbonyl chemistry on an ABI 43 1A peptide synthesizer (Applied Biosystems, Inc., Foster City, Calif.), then purified by reverse-phase high performance liquid chromatography. The purity and identity of the product were confirmed by determining molecular mass by electrospray mass spectrometry.

Immunization Methods.

Mice were immunized with 50 μg of the indicated peptide in incomplete Freund's adjuvant (1SA51, Seppic Inc., Paris France) at weeks 0, 3, and 7 and bled at weeks 2, (bleed 1 after boost 1), week 5 (bleed 2 after boost 2) and week 8 (bleed 3 after boost 3). Immune responses were seen after bleed 2 in most animals and data are reported from bleeds 2 and 3.

Guinea pigs were immunized intranasally with 200 μg of C4-V3 peptide in saline with 1 μg of cholera toxin as adjuvant as described. Guinea pigs were immunized on day 0, day 14 and day 21 and serum samples before and 1 week following each immunization obtained by cardiac puncture.

ELISA Assay.

Anti-HIV env peptide ELISA assays were performed as previously described (Haynes et al, J. Immunol. 151:1646-1653 (1993), Haynes et al, AID Res. Human Retroviruses 11:211-221 (1995)).

Splenocyte Proliferation Assay.

Mouse splenocyte proliferation assay using ³H-thymidine incorporation was performed as previously described (Haynes et al, AID Res. Human Retroviruses 11:211-221 (1995)).

Neutralizing Antibody Assays.

Assays for ability of anti-HIV antisera to neutralize HIV were performed as described (Palker et al, J. Immunol. 142:3612-3619 (1989), Haynes et al, Trans. Am. Assoc. Physician 106:31-41 (1993), Haynes et al, J. Immunol. 151:1646-1653 (1993), Haynes et al, AID Res. Human Retroviruses 11:211-221 (1995)).

NMR Spectroscopy.

Peptides were dissolved to 4 mM in a solution of 90% ¹H₂O, 10% ²H₂O, 20 mM NaCl, 5 mM KH₂PO₄, 1 mM sodium azide, 0.5 mM sodium 3-(trimethylsilyl) propionate, at a pH of 4.2. The methyl resonance of the latter component served as a chemical shift reference.

Spectra of samples prepared in this way were acquired with a Varian Unity 500 MHz spectrometer at a temperature of 278 K. The lock signal was from deuterium in the sample. The following two-dimensional spectra were obtained: (a) double-quantum-filtered correlation spectroscopy (DQF-COSY) (Piantini et al, J. Am. Chem. Soc. 104:6800-6801 (1982), Rance et al., Biochem. Biopjys. Res. Cornmun. 117:479-485 (1983)); (b) total correlation spectroscopy (TOCSY) (Bax et al, J. Magn. Reson. 65:355-360 (1985), Levitt et al, J. Magn. Reson. 47:328-330 (1982)) with a mixing time of 150 ins; and (c) nuclear Overhauser exchange spectroscopy (NOESY) (Jeener et al, J. Phys. Chem. 71:4546-4553 (1979)) with a mixing time of 300 ins. Water resonance was suppressed by selective saturation during the relaxation delay, and, for NOESY, during the mixing period. The spectral width was 6700 Hz, with the indirectly acquired dimension collected as 750 (COSY), 512 (TOCSY), or 350 (NOESY) complex increments; and the directly acquired dimension containing 1024 complex points. Data were processed with FELIX 2.3 software (Biosyrn, San Diego, Calif.). Directly acquired free-induction decays were corrected for base-line offset. Decays in both dimensions were multiplied by a sinebell-squared function (phase shifted by 75°) and zero-filled to 2048 points before Fourier-transformation.

Peptide Membrane Binding Assay.

Peptides at 10 ng/ml were incubated with 106 peripheral blood mononuclear cells for 1 hour at 4° C., washed ×3 with phosphate buffered saline PHz 7.0, contained 0.1% sodium azide, then incubated guinea pig anti-HIV 89.6 V3 antisera (xlhr) (Liao et al, J. Virol. 74:254-263 (2000)), wash as above and then incubated with FITC-conjugated goat anti-guinea pig IgG. After a final wash as alone, the cells were analyzed for the relative amount of peptide bound to either PB lymphocytes or PB monocytes as reflected in the mean fluorescent channel (MFC) of reactivity of the anti-HIV 89.5 V3 antisera.

### Results

Anti-gp120 V3 Antibody Responses Following Immunization of Mice with C4-V3RF, C4_{E9V}-V3RF(A), C4_{E9G}-V3RF(A) and C4_{K12E}-V3RF(A) Peptides.

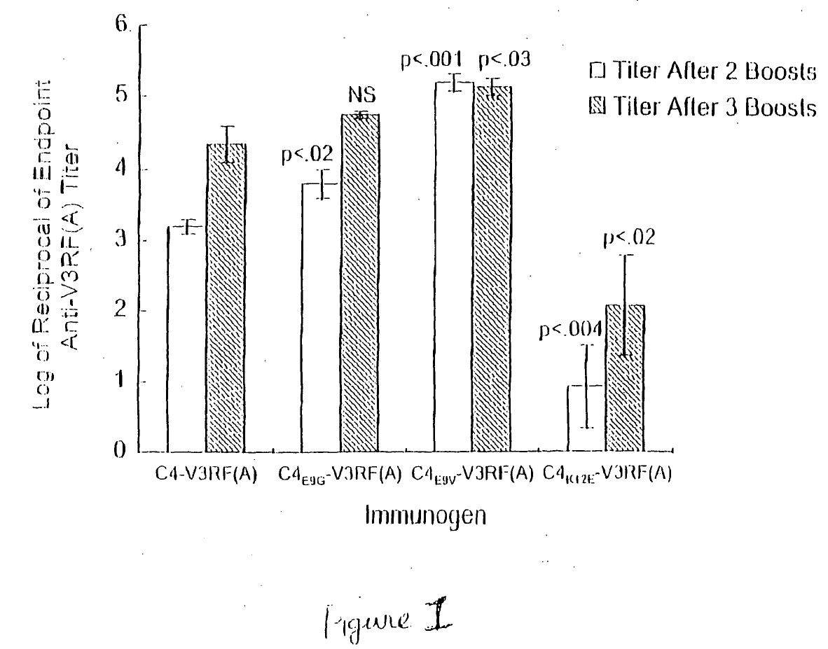

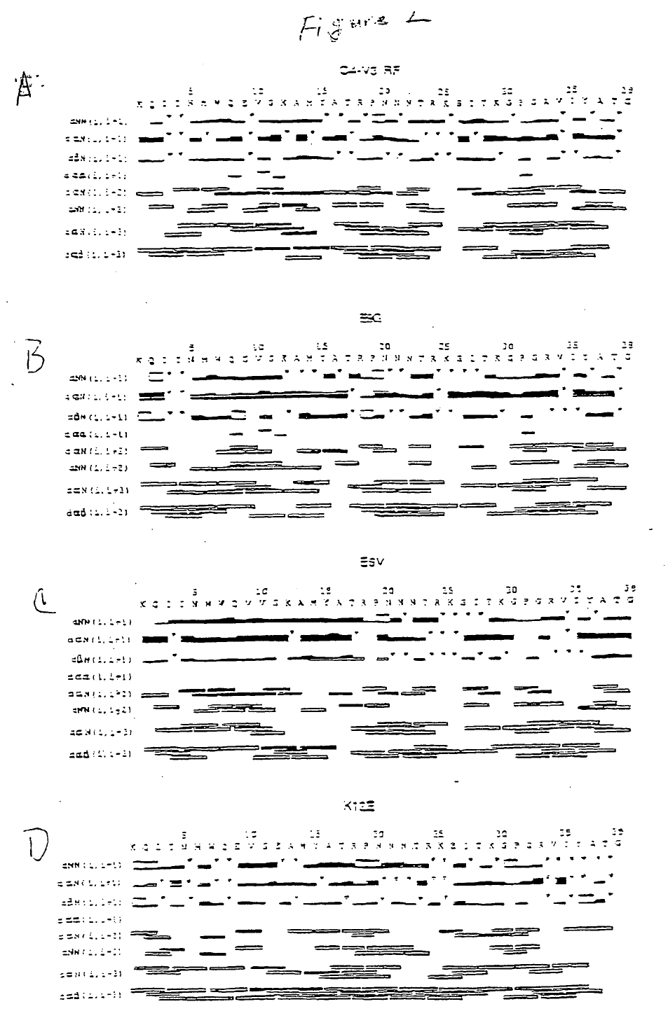

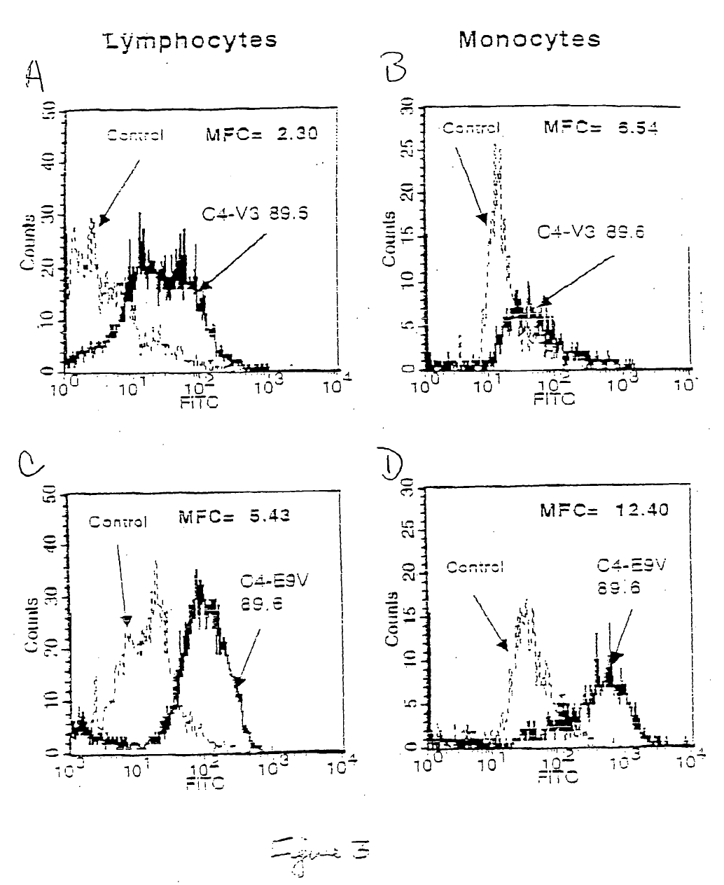

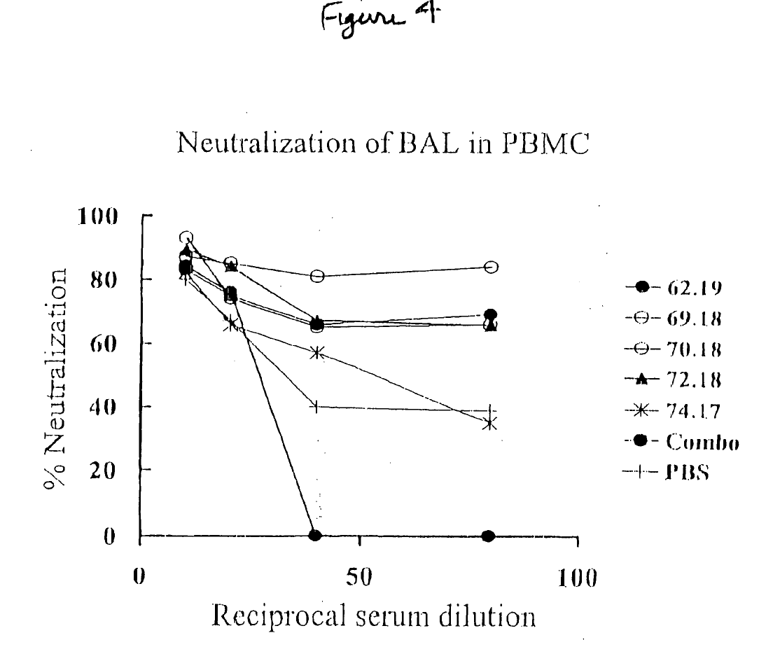

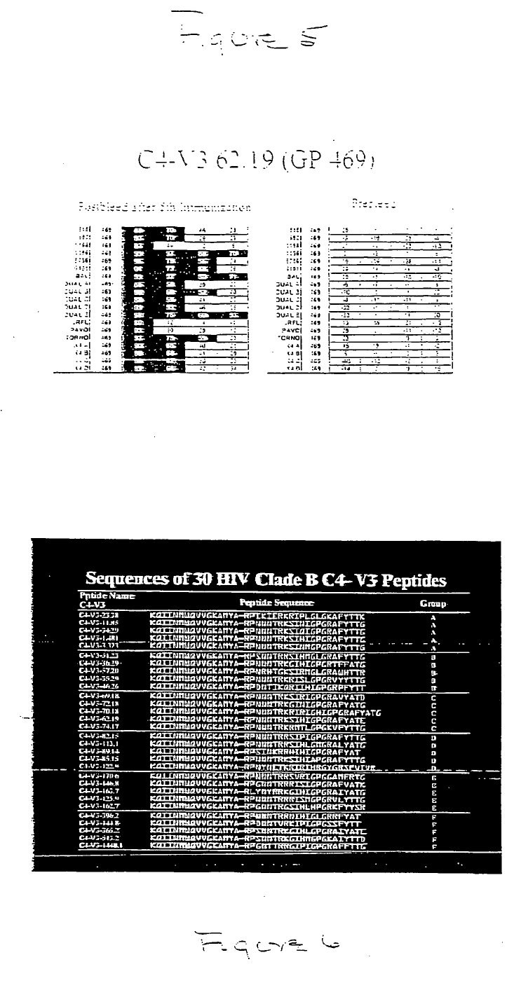

First, the ability of C4-V3HIVRF variants to modulate the immunogenicity of the peptide with regard to antibodies to the V3 portion of the C4-V3 immunogen were assayed. The results (FIG. 1, Table 2) show differences among the four peptides in their ability to induce anti-HIVRF V3 antibody responses. Sera from C4_{E9V}-V3RF(A)-immunized mice had a log higher anti-V3 antibody titer than either mice immunized with the native C4-V3RF(A) peptide or the C4E9V-V3RF(A) peptide variant. After one immunization, no anti-V3RF antibody response was seen in mice immunized with either C4-V3RF(A), C4_{E9G}-V3RF(A), or C4_{K12E}-V3RF(A) peptides. However, after only one immunization with 50 μg of the C4_{E9V}-V3 peptide, the geometric mean titer to V3RF(A) peptide was 1:5012 (n=3 mice), with titers of 1:3200, 1:3200 and 1:12,800 in each of the three mice tested, respectively. Thus, the E9V C4-V3RF(A) variant induced a higher titer and earlier anti-gp 120 V3 antibody responses than the other C4-V3RF(A) peptides tested. After 2 boosts, C4_{E9V}-V3RF(A)-immunized mice had 2 logs higher anti-V3 antibody responses than did C4-V3RF(A) immunized mice (FIG. 1, Table 2).

TABLE 2
Comparison of the Ability of C4-V3 Peptides To Induce HIV gp120 Anti-C4 and Anti-V3 Antibodies in Balb/c Mice

| | Number of | Peptide on Plate in ELISA For Anti-Peptide Antibody Assay | | | | | |
|---|---|---|---|---|---|---|---|
| | Animals | C4 | V3RF(A) | C4-V3RF(A) | C4E9G-V3RF(A) | C4E9V-V3RF(A) | C412EV3RF(A) |
| Peptide Immunogen | Geometric Mean Titer | | | | | | |
| C4-V3RF(A) | 6 | 2 | 1,584 | 2,239 | 1,195 | 1,584 | 1,412 |
| C4_{E9G}-V3RF(A) | 6 | 2 | 6,310 | 7,079 | 5,623 | 3,162 | 3,548 |
| C4_{E9V}-V3RF(A) | 5 | 14 | 151,356 | 131,825 | 87,096 | 87,096 | 114,815 |
| C4_{K12E}-V3RF(A) | 6 | 1 | 8 | 8 | 1 | 3 | 3 |

Data represent the reciprocal of endpoint dilutions at which the E/C was 3.0 in anti-peptide ELISA after two immunizations.

The C4_{K12E}-V3RF(A) peptide variant induced anti-V3 antibody responses 3 logs lower than the C4-V3RF(A) peptide after 2 immunizations (FIG. 1, Table 2). Thus, single amino-acid replacements in the C4 T helper region had extraordinary effects on immunogenicity of the HIVRF gp120 V3 domain.

Comparison of the Ability of C4-V3RF(A) Peptides to Induce Anti-HIV gp120 Peptide 3H-Thymidine Incorporation in Splenocytes from Naive and Peptide-Immunized Mice.

Next, C4-V3 peptides were tested for their ability to stimulate proliferation of splenocytes from peptide-immunized mice. Balb/c mice were sacrificed after the third peptide immunization and their splenocytes assayed for the ability to proliferate to PHA and to each peptide type (Table 3). It was found that C4-V3RF(A), C4_{E9V}-V3RF(A), and C4_{K12E}-V3RF(A) peptides all induced in vitro proliferative responses to the immunizing peptides, whereas the C4_{E9G}-V3RF(A) variant peptide did not induce proliferative responses in E9G-primed mice significantly over responses of naive mice (Table 3). Regarding the ability of the E9V peptide variant to induce earlier and greater anti-V3 antibody responses compared to the other peptides tested, the C4_{E9V}-V3RF(A) peptide-primed splenocytes for proliferation to the immunizing peptide only minimally better than did each of the other three peptides (Table 3). Thus, altered induction of T helper cell proliferative responses did not explain the differences in peptide immunogenicity.

TABLE 3
Comparison of the Ability of C4-V3 Peptides To Induce Anti-HIV gp120 Peptide ³H-Thymidine
Incorporation in Splenocytes from Naïve and Immunized Mice

| | Peptide Used As Stimulator in ³H-Thymidine Incorporation Assay | | | | | | |
|---|---|---|---|---|---|---|---|
| | N | C4 | V3RF(A) | C4-V3RF(A) | C4_{E9G}-V3RF(A) | C4_{E9V}-V3RF(A) | C4_{K12E}-V3RF(A) |
| Peptide Immunogen | Mean ± SEM CPM per 10⁶ Splenocytes in Culture | | | | | | |
| None (Naïve | 6 | 613 ± 322 | 408 ± 140 | 149 ± 84 | 114 ± 85 | 74 ± 47 | 187 ± 165 |
| Balb/c) | | | | | | | |
| C4-V3RF(A) | 6 | 2,289 ± 1,332 | 955 ± 353 | 8,390 ± 1,424 a | 8,067 ± 1,728 | 6,242 ± 1,787 | 6,198 ± 1,343 |
| C4_{E9G}-V3RF(A) | 6 | 408 ± 95 | 708 ± 325 | 2,103 ± 1,170 | 3,559 ± 2,310 b | 988 ± 340 | 1,101 ± 399 |
| C4_{E9V}-V3RF(A) | 5 | 84 ± 52 | 1,463 ± 473 | 933 ± 4,528 | 11,743 ± 3,830 | 24,824 ± 5,581 c | 10,269 ± 3,592 |
| C4_{K12E}-V3RF(A) | 6 | 3,430 ± 2,796 | 4,417 ± 2,217 | 8,670 ± 3,865 | 13,237 ± 8,563 | 7,513 ± 2,951 | 12,644 ± 4,138 d |

Data represent peak 3H-thymidine responses at 7 days.
CPM = CPM experimental − experimental − experimental control.
a p < .001 vs naïve mice; p = NS vs C4-V3RF(A) or C4K12E-V3RF(A) stimulated C4K12E-V3RF(A) immunized splenocytes.
b p = NS vs naïve mice.
c p < .001 vs naïve mice.
d p < .02 vs naïve mice.

The lower antibody titer induced by the C4_{K12E}-V3 peptide against V3RF(A) was not an artifact attributable to lack of ability of the V3 peptide not binding to the ELISA plate, as sera from C4_{E9V}-V3RF(A)-induced antisera had high reactivity to the V3RF(A) peptide on the ELISA plate. Similarly, the C4_{K12E}-V3RF(A) peptide could bind anti-V3RF antibody, as multiple antisera raised against C4-V3 peptides bound the C4_{K12E}-V3 variant (Table 2).

Antibody levels to the C4 region were also tested. The C4 region induced only a minimal antibody response compared to the V3 region, with all the C4-V3 peptides tested (Table 2).

Anti-gp 120 V3 Antibody Responses Following Immunization of Guinea Pigs.

Next, 2 guinea pigs were immunized each with 200 μg of C4-V3RF(A), C4_{E9G}-V3 RF(A), C4_{E9V}-V3 RF(A) or C4_{K12E}-V3 RF(A) peptide intranasally with 1 μg cholera toxin adjuvant in saline. Intranasal immunization of peptides with cholera toxin has been previously shown to result in CTL and titers of anti-peptide antibody similar in levels to titers induced by initial antigens administered subcutaneously or intramuscularly in oil in water adjuvants such as complete and incomplete Freund's adjuvant. In addition, it was desirable to determine the ability of C4-V3 peptides in an aqueous-solution (such as in saline for intranasal immunization) to induce anti-HIV antibody responses in order to correlate reactivity of antibodies generated against peptide in an aqueous adjuvant with peptide conformers solved in an aqueous solution. Finally, there was interest in determining if the amino acid substitutions in the C4 region conferred on the C4-V3 peptides the same pattern of immunogenicity as seen in oil in water adjuvant in mice.

It was found that after 2 immunizations the C4-V3 RF(A) peptide induced a mean anti-HIV peptide antibody titer of 3981, peptide induced titers of 1 log (GMT=31,623) higher. As in mice, substituting the Glu (E) for Lys (K) at position 12 in the C4 peptide abrogated peptide immunogenicity in guinea pigs (GMT=16) (Table 4).

TABLE 4
Titers of C4-V3 HIV Envelope Antibodies Induced by
C4-V3RF(A) Peptides in Guinea Pigs

| Immunizing Peptide | Titer Against Immunizing Peptide* |
|---|---|
| C4-V3RF(A) | 3,981 |
| C4-_{E9G}-V3RF(A) | 2,818 |
| C4-_{E9V}-V3RF(A) | 31,623 |
| C4-_{K12E}-V3RF(A) | 16 |

*Data represent the mean titers from 2 animals after 2-3 immunizations intranasally with 400 ug of the indicated peptide formulated in saline with cholera toxin as an adjuvant.

Ability of Antibodies Against C4-V3 Peptides to Induce Neutralizing Antibodies.

In order to induce high levels of neutralizing antibodies with C4-V3 peptides, usually 5 immunizations are given (Palker et al, J. Immunol. 142:3612-3619 (1989), Haynes et al, J. Immunol. 151:1646-1653 (1993), Palker et al, Proc. Natl. Acad. Sci. USA 85:1932-1936 (1988), Liao et al, J. Virol. 74:254-263 (2000)). The guinea pig sera from the experiment presented in Table 4 were tested for ability to neutralize HIVRF. It was found that one sera from the C4-V3RF(A)-immunized animals (after 3 injections) had a neutralizing antibody titer of 1:40 against HIVRF, while one animal of the C4_{E9V}-V3RF(A)-injected animals had a neutralizing titer of 1:340 after only 2 injections. Thus, antibodies induced by the C4_{E9V}-V3RF(A) peptide can bind to native gp120 and neutralize HIVRF.

Inability of the C4-E9V-RF(A) Sera to Bind to gp120 from HIV_{IIIB}.

The V3 loop sequence of HIV_{IIIB} is different from that of HIVRF, and thus HIVRF anti-V3 neutralizing antibodies do not neutralize HIV_{IIIB}. To determine if any antibodies were generated by any of the C4-V3RF(A) variant peptides, all the mouse sera in Table 2 were tested, as were the guinea pig sera in Table 4, for the ability to bind to native recombinant HIV_{IIIB} gp120 in ELISA. Since anti-HIVRF V3 antibodies do not bind to the HIV_{IIIB} V3 loop, any binding activity of these anti-C4-V3 sera would be to the C4 region of HIV_{IIIB}, which is conserved between HIV_{IIIB} and HIVRF. No binding of any mouse or guinea pig anti-C4-V3 sera to HIV_{IIIB} gp120 was seen, indicating the inability of these peptides to induce antibodies against the native gp120 C4 region.

Conformational Propensities of C4-V3 RF Sequence Variants in Aqueous Solution.

Next, the peptides were examined by NMR to determine whether conformational changes had been induced by amino-acid sequence alteration. It was hypothesized that specific amino-acid substitutions in the C4 segment would lead to a decrease in the tendency of this region to adopt transient helical conformations. To test this hypothesis, each of the four peptides, C4-V3RF and variants E9G, E9V and K12E, was subjected to ¹H NMR spectroscopy to assign resonances and to analyze nuclear Overhauser effects between hydrogen nuclei on separate residues.

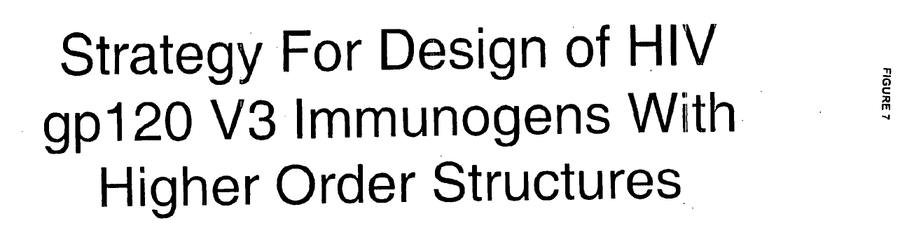

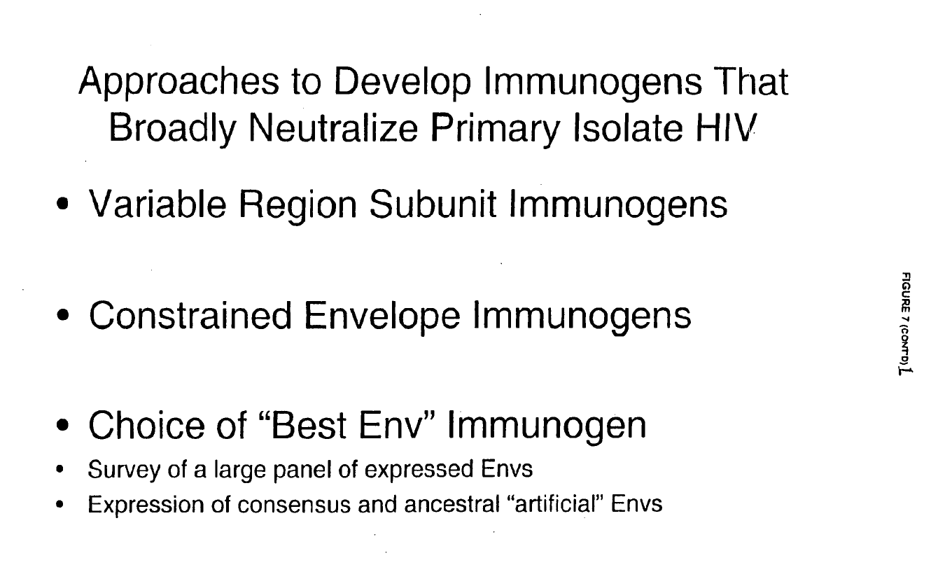

Resonance assignments for nearly all ¹H were determined from TOCSY, DQF-COSY, and NOESY spectra by standard methods (Wuthrich, NMR of Proteins and Nucleic Acids, John Wiley and Sons, New York (1986)), and are shown in FIG. 2. The value of the chemical shift for a main-chain ¹H, for example, the a carbon C^{a}H, is correlated with secondary structure in the case of proteins or well structured peptides (Wishart et al, J. Mol. Biol. 222:311-333 (1991)). Hence, strong tendencies among C4-V3RF peptides to adopt secondary structure in solution may be manifested in chemical shift values. This was examined by calculating for each peptide the difference in chemical shift between the C—H of each residue and a shift value representing the average for all secondary structures in proteins (Wishart et al, J. Mol. Biol. 222:311-333 (1991)). In no peptide were there stretches of sequence with high or low values of the chemical shift difference that would be evidence of stable secondary structure, for example helix or β strand.

NMR parameters such as chemical shift and coupling constants are often insensitive indicators of weak preferences for particular conformations since their values are the average of the entire population, thus obscuring the contribution of a slight bias for populating certain conformations. The nuclear Overhauser effect (NOE) is often more sensitive at revealing conformational propensities because it may give rise to a unique signal, although weak, on a background consisting only of random noise. Hence, NOESY spectra of C4-V3RF and its variants were characterized to identify each signal and evaluate its relative intensity. Sequential and medium range NOEs involving main-chain NH or CaH are listed in FIG. 2. These NOEs and the possible conformational propensities they represent are discussed as follows for C4_{E9G}-V3RF(A) and C4_{E9V}-V3RF(A). Variant C4_{K12E}-V3RF(A)K12E is discussed separately below because it was studied under different conditions.

In terms of overall conformation, all four peptides showed NOE patterns suggesting no tendency to adopt stable structure. For example, sequential daN(i, i+1) and dNN(i, i+1) NOEs were usually both present for each sequential pair of residues, with the former typically more intense, indicating that f and j main-chain dihedral bond angles varied and maintained on average an extended conformation (Dyson et al, Ann. Rev. Biophys. Chem. 20:519-538 (1991)). Also the absence of long range NOEs [(i, i+5) or greater] and the few and generally weak medium-range NOEs suggested no significant population of higher order structure.

However, the fact that some medium range NOEs were detected is evidence of propensity to adopt non-random conformations in certain regions (Dyson et al, Ann. Rev. Biophys. Chem. 20:519-538 (1991)). Although only one mixing time was used for NOESY spectra (300 ins), previous studies of a related C4-V3 RF peptide (de Lorimier et al, Biochemistry 33:2055-2062 (1994)) showed that medium range NOEs were still observable at shorter (75 and 150 ins) mixing times. Hence, the NOEs indicating medium range interactions are not likely due to spin-diffusion.

Within the C4 segment C4-V3RF and C4_{E9V}-V3RF(A) showed numerous medium range NOEs which are consistent with a tendency of this region to populate nascent helical conformations. The presence of contiguous or overlapping daN(i,i+2) NOEs from Trp⁷ to Tyr¹⁵ (C4-V3RF) and from Ile⁴ to Lys¹² (E9V) indicates a propensity for nascent helical turns in these regions (Dyson et al, Ann. Rev. Biophys. Chem. 20:519-538 (1991), Dyson et al, J. Mol. Biol. 201:201-217 (1988)). A dNN(i,i+2) NOE in this region in C4-V3 RF (between Lys¹² and Met¹⁴) is also consistent with main-chain f and j dihedral angles representative of helical turns (Dyson et al, Ann. Rev. Biophys. Chem. 20:519-538 (1991)). C4-V3 RF shows three consecutive daN(i,i+3) NOEs from residues Val¹⁰ to Tyr¹⁵, which is highly indicative of full helical turns. The presence of equivalent NOEs in E9V could not be ascertained due to overlap with other NOEs. However both C4-V3RF and E9V show two dab(i,i+3) NOEs, between Val¹⁰ and Ala¹³ and between Ala¹³ and Met¹⁴. This type of NOE is also highly suggestive of full helical turns in these regions of C4.

Variant C4_{E9G}-V3RF(A) on the other hand showed no evidence, in terms of medium range NOEs, for preferential population of certain conformations in C4. This absence of medium range NOEs was not due merely to ambiguities caused by signal overlap, because there were at least five positions where an NOE was unambiguously absent in C4_{E9G}-V3RF(A), but present in the parent peptide C4-V3 RF. Thus, the E to G substitution in the C4 peptide appeared to prevent helical conformer formation in the peptide.

In the V3 segment of the three peptides, C4-V3 RF, C4_{E9G}-V3RF(A) and C4_{E9V}-V3RF(A), were medium range NOEs suggesting preferred solution conformations in certain RE regions. All three peptides showed evidence of a reverse turn in the sequence Arg¹⁸-Pro¹⁹-Asn²⁰-Asn²¹, where these residues comprised positions 1 to 4, respectively, of the turn. The NOE pattern consistent with a reverse turn included a weak dNd(i,i+1) between Arg¹⁸ and Pro¹⁹, undetectable ddN(i,I+1) between Pro¹⁹ and Asn²¹, weak dad(i,i+1) between Arg¹⁸ and Pro¹⁹, strong daN(i,I+1) between Pro¹⁹ and Asn²⁰, and detectable daN(i,i+2) between Pro¹⁹ and Asn²¹ (Dyson et al, J. Mol. Biol. 201:161-200 (1988)). The detection of the weak dNd(i,i+1) NOE (Arg¹⁸ to Pro¹⁹) suggested that a Type I turn may be the preferred conformation (Dyson et al, J. Mol. Biol. 201:161-200 (1988)).

All three peptides also showed evidence of preferred conformers at the sequence Ser²⁶-Ile²⁷-Thr²⁸-Lys²⁹. There were two consecutive daN(i,i+2) NOEs, between Ser²⁶ and Thr²⁸ and between Ile²⁷ and Lys²⁹, as well as medium range NOEs not shown in FIG. 2. The latter included a dbN(i,i+2) NOE between Ser²⁶ and Thr²⁸, and a dba(i,i+2) NOE between these same residues. The conformational preferences giving rise to these NOEs did not fit a typical secondary structure, and suggested an unusual turn that placed the side-chain of Ser²⁶ in close proximity to the main-chain groups of Thr²⁸. This type of conformation has been described as a kink in the context of a helical region (Osterhout et al, Biochemistry 28:7059-7064 (1989)).

A third conformational feature in the V3 segments of C4-V3RF, C4_{E9V}-V3RF(A) and C4_{E9G}-V3RF(A)occurred in the sequence Gly³⁰-Pro³¹-Gly³²-Arg³³. In E9G the NOEs between these residues resembled the pattern described above that was consistent with a reverse turn (Dyson et al, J. Mol. Biol. 201:161-200 (1988)). This included a weak dNd(i,i+1) NOE between Gly³⁰ and Pro³¹, a weak ddN(i, i−I−i) NOE between Pro³¹ and Gly³², a weak dad(i,i+1) NOE between Gly³⁰ and Pro³¹, a strong daN(i,i+1) NOE between Pro³¹ and Gly³², and a detectable daN(i,i+2) NOE between Pro³¹ and Arg³³. In the C4-V3R peptide, the pattern of (i,i+1) NOE intensities was the same but no daN(i,i+2) NOE was detected between Pro³¹ and Arg³³. Instead a daN(i,i+2) NOE was detected between Gly³⁰ and Gly³². And in C4-E9V V3RF, both daN(i,i+2) NOEs, Gly³⁰ to Gly³² and Pro³¹ to Arg³³, were detected. These data raised the possibility that two independent turn-like conformational preferences occurred in this region of V3. The fact that a Pro³¹-Arg³³ daN(i,i+2) NOE was unambiguously absent in C4-V3RF, and that a daN(i, I+2) NOE between Gly³⁰ and Gly³² was also unambiguously absent in C4_{E9G}-V3RF(A), in spite of sequence identity in all three peptides, may be related to the weak intensity of these NOEs. Being close to the level of noise intensity, there is a possibility that one or both NOE signals on either side of the spectrum will not be detected, thus disallowing the given NOE to be scored as such.

Another region in V3 where conformational preferences could be inferred from NOEs occurs in residues Val³⁴-Ile³⁵-Tyr³⁶. In all three peptides NOEs were observed between the upfield methyl resonance (˜0.67 ppm) of Val³⁴ and the ring hydrogens, both dH and eH, of Tyr³⁶. Weaker NOEs are also seen between the downfield methyl resonance (˜0.89 ppm) of Val³⁴ and the ring hydrogens of Tyr³⁶. Further evidence of close proximity between the side-chains of Val³⁴ and Tyr³⁶ was the fact that the two methyl resonances of the former had disparate chemical shifts, compared to Val¹⁰, consistent with a ring-current shift induced by the aromatic side-chain of Tyr. One peptide, C4-V3RF(A) had another NOE in this region, daN(i,i+2) between Ile³⁵ and Ala³⁷, that was unambiguously absent in the C4_{E9G}-V3RF(A) and C4_{E9V}-V3RF(A) peptides. This observation likely represented a poorly populated conformation, perhaps related to that which gives rise to the Val³⁴-Tyr³⁶ side-chain interaction, or from an independent conformational propensity.

Substitution of Lys¹² with Glu yielded a poorly immunogenic peptide (C4_{K12E}-V3RF(A)) that, interestingly had solution properties different from the other three peptides studied. Under the conditions used for NMR studies of other C4-V3 peptides, the solution of the C4_{K12E}-V3RF(A) peptides was highly viscous, and viscosity increased with pH in the vicinity of pH 4, implicating ionization of the Glu¹² side-chain in this phenomenon. NMR spectra of K12E at 278 K in aqueous buffer showed a much lower signal-to-noise ratio than the other three peptides. Increasing the temperature to 318 K or decreasing the pH to 3.5 yielded improved but still inadequate signal. Suitably high signal for resonance assignment and NOE analysis was obtained at 318 K, pH 3.5, 20% v/v trifluoroethanol (d₃). Even under this condition the NOEs for the C4_{K12E}-V3RF(A) were less intense than for other peptides.

NOE connectivities in the C4 segment of C4_{K12E}-V3RF(A) (FIG. 2) show evidence of nascent helical turns in the region between Ile³ and Gly¹¹ as inferred from dNN(i, i+2) and daN(i,i+2) NOEs. The stretch from Val¹⁰ to Thr¹⁷ has two daN(i,i+3) and two dab(i,i+3) NOEs suggesting the presence of a significant population with full helical turns. Within the V3 segment only two medium range NOEs are observed, both daN(i,i+2). Neither corresponds to NOEs observed in the other three peptides, but both NOEs involve residues of the Ser²⁶-Ile²⁷-Thr²⁸ sequence, for which there is evidence of conformational preferences in the other three peptides. A dbN(i,i+2) NOE between Ser²⁶ and Thr²⁸, observed in C4_{E9V}-V3RF(A)) and C4_{E9G}-V3RF(A), is also observed in the K12E peptide. Also observed are NOEs between the side-chains of Val³⁴ and Tyr³⁶. Hence the conformations giving rise to these two features are at least partially preserved under the solution conditions employed for K12E. Differences in the V3 segment between K12E and all of the other three peptides include the absence of detectable daN(i, 1+2) NOE between Pro¹⁹ and Asn²¹ and between Ser²⁶ and Thr²⁸. The failure to detect these NOEs may be due to the overall weaker signals of this sample, or to depopulation of the relevant conformations by the solution conditions.

### EXAMPLE 2

The peptides in Table 7 have been studied in groups of 5 peptides as indicated in Table 9, and each group of 5 peptides has been injected into each of three guinea pigs in Freund's complete then incomplete adjuvant. After 4 immunizations, the animals were bled, and heat inactivated serum was pooled from each animal or tested separately as indicated in Table 8, for the ability to neutralize HIV. Single numbers per group indicate that the results are those of pooled sera from the group. Individual results per animal indicate that each serum was tested individually. Table 8 shows that all the sera neutralized to varying degrees the T cell line adapted HIV isolate MN and poorly neutralized the TCLA HIV isolate IIIB. Regarding the rest of the isolates in Table 8, all of which are HIV primary isolates (89.6, BAL ADA, SFI 62, 5768, QHO515, PVO, JRFL, BX08, 6101, SS1196), Group C sera from C4-V3 subtype B peptides neutralized 4/11 (36%) and Group F sera from subtype B peptides neutralized 5/11 primary isolates (45%). FIG. 4 shows that for the HIV CCR5 utilizing primary isolate, BAL, that the individual peptides in the 5-valent mixture absorbed out the neutralizing activity against HIV BAL to varying degrees, whereas the mixture of all the peptides completely absorbed out the neutralizing activity.

TABLE 8
Neutralization Of HIV-1 Isolates By Sera From
Guinca Pigs Immunized With C4-V3 Clade B
Peptides

| Animal | Immunogen | HIVMN# | HIVIIIB# | SHIV89 6# | SHIV89.6# | HIVBAI.* | ADA* |
|---|---|---|---|---|---|---|---|
| 477 | A | 2,258 | 0 | 96 | 35 | 0 | 0 |
| 478 | A | 1,157 | 0 | NA | | 0 | |
| 479 | A | 4,612 | 68 | NA | | 0 | |
| 480 | B | 1158 | 0 | NA | 27 | 0 | 0 |
| 481 | B | 1,714 | 0 | NA | | 84 | |
| 482 | B | 4,241 | 0 | 02 | | 0 | |
| 483 | C | 969 | 0 | 112 | 97 | 95 | 0 |
| 484 | C | 806 | 0 | 20 | | 84 | |
| 485 | C | 542 | 0 | 226 | | 80 | |
| 486 | D | 1,488 | 0 | NA | 98 | 0 | 0 |
| 487 | D | 2,184 | 0 | NA | | 80 | |
| 488 | D | 575 | 0 | NA | | 0 | |
| 489 | E | 3,223 | 0 | NA | 255 | 88 | 0 |
| 490 | E | NA | 0 | NA | | 0 | |
| 491 | E | 519 | 0 | NA | | 81 | |
| 492 | F | NA | 0 | NA | 0 | NA | 0 |
| 491 | F | 910 | 0 | NA | | 91 | |
| 494 | F | 1,159 | 35 | NA | | NA | |

| Animal | SF162* | S768* | QH0515* | PV0* | IRFL* | HX118* | 6101* | SS1196* |
|---|---|---|---|---|---|---|---|---|
| 477 | 90 | 0 | 0 | 0 | 0 | 0 | 0 | 85 |
| 478 | | | | | | | | |
| 479 | | | | | | | | |
| 480 | 96 | 0 | 0 | 0 | 0 | 0 | 0 | 0 |
| 481 | | | | | | | | |
| 482 | | | | | | | | |
| 483 | 99 | 0 | 0 | 0 | 0 | 86 | 0 | 0 |
| 484 | | | | | | | | |
| 485 | | | | | | | | |
| 486 | 98 | 0 | 0 | 0 | 0 | 94 | 0 | 0 |
| 487 | | | | | | | | |
| 488 | | | | | | | | |
| 489 | 92 | 0 | 0 | 0 | 0 | 0 | 0 | 0 |
| 490 | | | | | | | | |
| 491 | | | | | | | | |
| 492 | 84 | 0 | 0 | 0 | 0 | 91 | 94 | 88 |
| 491 | | | | | | | | |
| 494 | | | | | | | | |

#Assay [symbol] are [symbol] dilutions at which 50% of [symbol] -2 cells were protected [symbol] included [symbol] illing as measured by neutral [symbol] * [symbol] in pH synthesis relative to the amount of p24 synthesised in the presence of corresponding [symbol] Values [symbol] are positive
NA—Not available

[symbol] Pig Immunization Protocol Part 2
Immunization with a group of 5 peptides
Peptide Name

| | | | |
|---|---|---|---|
| C4 V3 peptide | Peptide Sequence | Code | [symbol] [symbol] |
| C4 V3 [symbol] | KQIINMWQVVGKAMYA- [symbol] | A | [symbol] |
| C4 V3 [symbol] | KQIINMWQVVGKAMYA- [symbol] | A | |
| C4 V3 [symbol] | KQIINMWQVVGKAMYA- [symbol] | A | |
| C4 V3 [symbol] | KQIINMWQVVGKAMYA- [symbol] | A | |
| C4 V3 [symbol] | KQIINMWQVVGKAMYA- [symbol] | A | |
| C4 V3 [symbol] | KQIINMWQVVGKAMYA- [symbol] | B | [symbol] |
| C4 V3 [symbol] | KQIINMWQVVGKAMYA- [symbol] | B | |
| C4 V3 [symbol] | KQIINMWQVVGKAMYA- [symbol] | B | |
| C4 V3 [symbol] | KQIINMWQVVGKAMYA- [symbol] | B | |
| C4 V3 [symbol] | KQIINMWQVVGKAMYA- [symbol] | B | |
| C4 V3 [symbol] | KQIINMWQVVGKAMYA- [symbol] | C | [symbol] |
| C4 V3 [symbol] | KQIINMWQVVGKAMYA- [symbol] | C | |
| C4 V3 [symbol] | KQIINMWQVVGKAMYA- [symbol] | C | |
| C4 V3 [symbol] | KQIINMWQVVGKAMYA- [symbol] | C | |
| C4 V3 [symbol] | KQIINMWQVVGKAMYA- [symbol] | C | |
| C4 V3 [symbol] | KQIINMWQVVGKAMYA- [symbol] | D | [symbol] |
| C4 V3 [symbol] | KQIINMWQVVGKAMYA- [symbol] | D | |
| C4 V3 [symbol] | KQIINMWQVVGKAMYA- [symbol] | D | |
| C4 V3 [symbol] | KQIINMWQVVGKAMYA- [symbol] | D | |
| C4 V3 [symbol] | KQIINMWQVVGKAMYA- [symbol] | D | |
| C4 V3 [symbol] | KQIINMWQVVGKAMYA- [symbol] | E | [symbol] |
| C4 V3 [symbol] | KQIINMWQVVGKAMYA- [symbol] | E | |
| C4 V3 [symbol] | KQIINMWQVVGKAMYA- [symbol] | E | |
| C4 V3 [symbol] | KQIINMWQVVGKAMYA- [symbol] | E | |
| C4 V3 [symbol] | KQIINMWQVVGKAMYA- [symbol] | E | |
| C4 V3 [symbol] | KQIINMWQVVGKAMYA- [symbol] | F | [symbol] |
| C4 V3 [symbol] | KQIINMWQVVGKAMYA- [symbol] | F | |
| C4 V3 [symbol] | KQIINMWQVVGKAMYA- [symbol] | F | |
| C4 V3 [symbol] | KQIINMWQVVGKAMYA- [symbol] | F | |
| C4 V3 [symbol] | KQIINMWQVVGKAMYA- [symbol] | F | |

It is important to be able to use T helper determinants with the V3 portion of the peptides shown in Table 7, both to expand the T helper activity in the immunogen, and in case any of the T helper peptides should be found to have any deleterious effects in the course of human trials. For example, it has recently been found in vitro that in culture of HIV and T cells, that the C4 portion of the C4-V3 peptide can augment HIV induced syncytium formation. However, peptides of this general design have been studied in vitro in HIV-infected humans (AIDS 12: 1291-1300, 1998) and no subjects developed a ≧10 fold change in plasma HIV RNA levels from baseline. Moreover, the primary use of these peptides is as an immunogen in HIV− subjects as a preventive vaccine, and not in doses that one would consider for therapy, which would be in milligram amounts daily. A T helper determinant from HIV gag, termed GTH1 with the sequence of Y K R W I I L G L N K I V R M Y S has been conjugated to the V3 of HIV MN and found to induce anti-HIV MN titers of 1:3200. Similarly, GTH1 conjugated to a V3 sequence of a HIV primary isolate DU179 induced antibodies that neutralized HIV MN (1:192) and neutralized the HIV primary isolate JR-FL (90% p24 reduction in PBMC cultures). Thus, the GTH1 T helper sequence can substitute for the C4 sequence in the peptides in Table 7.

Finally, a panel of monovalent serum from individual guinea pigs immunized with each of the peptides in Table 7 has been screened. Whereas most of the peptides in the list only induced neutralizing antibodies that neutralized 0 to 6 out of 19 primary isolates, 5 peptides were found that neutralized from 14 to 19 out of 19 primary isolates tested. These peptides were C4-V3 36.29, C4-V3 34.29, C4-V3 62.19, C4-V3 74.17, and C4-V3 162.7. The sequences of these peptides are all listed in Table 7.

Thus, sufficient breadth has been observed both in mixtures of C4-V3 peptides and in select individual peptides for the immunogen to be practical with regard to induction of neutralizing antibodies against HIV primary isolates. By performing the same immunization studies with the similarly designed HIV subtype (clade) C peptides in Table 6, that a similar immunogen(s) can be developed for HIV subtype C viruses.

While individual peptides can be used to achieve the breadth of neutralizing activity needed to protect against HIV primary isolates, advantageously, mixtures of multiple peptides are used, such as the combination of group C, or group F or the combination of C4-V3 36.29, C4-V3 34.29, C4-V3 62.19, C4-V3 74.17, and C4-V3 162.7 peptides described above.

### EXAMPLE 3

### HIV-1 Clade B V3-Based Polyvalent Immunogen

Anti-HIV gp120 V3 antibodies can neutralize some HIV primary isolates ((Hioe et al, Internat. Immunology 9:1281 (1997), Liao et al, J. Virol. 74:254 (2000), Karachmarov et al, AIDS Res. Human Retrovirol. 17:1737 (2001), Letvin et al, J. Virol. 75:4165 (2001)). The hypothesis for these studies was that sequence variation found among HIV primary isolates need not reflect the diversity of HIV serotypes, and antibodies can cross-react with groups of similar viruses. Data from comparison of NMR structures of several V3 loops and their immunogenicity patterns indicate that there are conserved higher order structures of the V3 that are similar in antigenicity regardless of primary amino acid heterogeneity (Vu et al, J. Virol. 73:746 (1999)).

1514 unique clade B V3 sequences in the Los Alamos National Laboratory HIV Database were analyzed by the following methods. Short antigenic domains were organized by protein similarity scores using maximum-linkage clustering (Korber et al, J. Virol. 68:6730 (1994)). This enabled visualization of clustering patterns as a dendritogram, and the splitting pattern in the dendritogram could be used to define clusters of related sequences. This method allows the use of several different amino acid scoring schemes. The amino acid substitution matrix of Henikoff and Henikoff was used which was designed to give amino acid substitutions well tolerated in conserved protein structural elements a high score, and those that were not, a low score (Henikoff and Henikoff, Protein Structure Function and Genetics 17:49 (1993)). Based on these clustering patterns, a variant was selected that was most representative of each group. Excluded were very rare, highly divergent sequences, and favored were sequences found in many different individuals. This method allowed for most of the unique V3 sequences to be within one or two amino acids from at least one of the peptides in the cocktail. Thus, 1514 clade B V3 sequences were clustered into 30 groups. The consensus peptide of each group was synthesized, purified to homogeneity by HPLC and confirmed to be correct by mass spectrometry. Each peptide was immunized into a guinea pig (GP) in-Incomplete Freunds Adjuvant (IFA), and each sera was tested after the fifth immunization by a single infection cycle neutralization assay preformed by ViroLogics, South San Francisco, Calif., or by a fusion from without HIV fusion inhibition assay using aldrithiol-2 inactivated HIV_{ADA}, HIV_{MN} and HIV_{AD8} virons (Rosio et al, J. Virol. 72:7992 (1998)).

The criteria established for acceptable neutralization of primary isolates was the ability of a serum to neutralize at least 25% of the HIV primary isolates tested. Using these criteria, 7 peptides were found that induced neutralizing antibodies against >25% of isolates tested. One of these peptides, peptide 62.19, neutralized 19/19 HIV primary isolates tested, even when the criteria were increased to greater than 80% neutralization vs. 50% neutralization (see FIG. 5 and Table 1).

When the sequences of 6 peptides that induced no (0/19) neutralization of the 19 primary HIV isolates were evaluated, it was found that they were all unusual sequences at the tip of the V3 loop, with sequences such as GLGR, GPGG, GLGK, GLGL, and GLGR present (see Table 10). Only 1 of the 19 isolates tested had one of the these V3 sequences, a GPGG sequence, that was not neutralized by the serum from the GPGG-immunized guinea pig. Therefore, one serologic defined group of Clade B HIV isolates may be defined by the primary amino acid sequences at the tip of the loop of GLGR, GPGG, GLGK, GLGL.

TABLE 10

Sequences of Peptides That Induced No
Neutralization at 50% Inhibition (All
Dilutions) Criteria

| | | |
|---|---|---|
| GP No. | Peptide No. | V3 Sequence(s) |

| | | |
|---|---|---|
| 447 | C4-V3 396.2 | RPNNNTRRNIHIGLGRRFYAT |
| 448 | C4-V3 170.6 | RPNNNTRRSVRIGPGGAMFRTG |
| 451 | C4-V3 23.38 | RPIKIERKRIPLGLGKAFYTTK |
| 458 | C4-V3 51.23 | RPSVNNTRRSIHMGLGRAFYTTG |
| 404 | C4-V3 57.20 | RPNRHTGKSIRMGLGLRAWHTTR |
| 432 | 396.2/170.6 | RRNIHIGLGRRF RRSVRIGPGGAM |

TABLE 11

Sequences of Peptides That Best Neutralized
Clade B Isolates at 50% Inhibition (All
Dilutions) Criteria

| | | |
|---|---|---|
| GP No. | Peptide No. | V3 Sequence(s) |

| | | |
|---|---|---|
| 436 | 69.18/146.8 | RKSIRIGPGRAV RRRISIGPGRAF |
| 442 | 1.481/85.15 | RKSIHIGPGRAF RKSIHIAPGRAF |
| 460 (B) | C4-V3 36.29 | RPNNNTRKGIHIGPGRTFFATG |
| 465 (A) | C4-V3 11.85 | RPNNNTRKSINIGPGRAFYTTG |
| 466 (A) | C4-V3 34.29 | RPNNNTRKSIQIGPGRAFYTTG |
| 467 (A) | C4-V3 1.481 | RPNNNTRKSIHIGPGRAFYTTG |
| 469 (C) | C4-V3 62.19 | RPNNNTRKSIHIGPGRAFYATE |
| 472 (C) | C4-V3 74.17 | RPNNNTRKRMTLGPGKVFYTTG |
| 475 (E) | C4-V3 162.7 | RPGNNTRGSIHLHPGRKFYYSR |

When the peptide sequences that induced neutralization of >25% of primary isolates were examined, it was found that the sequences were all similar and were all clustered around the Clade B V3 consensus sequence of IHIGPGRAFYTTG (see Table 11). However, not all peptides with this type of sequence induced good neutralizing antibodies-15 peptides had this type of sequence and did not induce good neutralizing antibodies. Thus, a “computer guided proteomic screen of the V3 loop” has been performed and V3 peptides have been identified that express higher order conformers that mirror the native functionally active motif of the V3 that is both available and capable of being bound by neutralizing antibodies. In particular, peptide 62.19 induced neutralizing antibodies against 19 of 19 HIV isolates. Expression of the consensus B V3 sequences in Table 11, and expression of certain of the unusual V3 sequences in Table 10, can define a “bivalent” clade B immunogen for use world wide where those sequences are present in the resident HIV quasispecies, likewise, the sequences shown in Table 12. Table 12 shows full V3 consensus sequences for the V3 loops of the indicated peptides. By placing these full length V3 loop sequences into a fill length HIV envelope gp120 or gp160/gp140 molecule, the ability of these peptides to induce neutralizing activity is transferred to the HIV envelope containing these sequences. Thus, for example, for the artificially designed consensus of consensus HIV envelope with less divergence from other HIV isolates compared to native HIV envelopes (Gaschen et al, Science 296: 2354-2360 (2002)), inclusion of one of the V3 sequences in Table 12 that has been shown to induce neutralizing activity against HIV primary isolates would augment the ability of the consensus of consensus artificial envelope to induce neutralizing antibodies. Further, expressing the V3 sequences in Table 12 would augment their immunogenicity by combining the V3 with other neutralizing sites on an immunogen (the intact envelope monomer or trimer).

Immunization with a replicating vector, expressing partial or entire (C to C) segments of these V3 loops, can be used to induce long lasting immunity to HIV.

TABLE 12
V3 Consensus Sequence

| | | |
|---|---|---|
| Name of | Total Seq | |
| peptide | in Database | Amino Acid Sequence |

| | | |
|---|---|---|
| 1.481 | 345 | SVEINCTRPNNNTRKSIHIGPGRAFYTTGEIIGDIRCAHCNISRA |
| 52.9C | 952 | SVEINCTRPNNNTRKSIHIGPGRAFYATERIIGDIRCAHCNISRT |
| 52.9Δ7 | — | SVEINCTRPNNNTRKSIHIGPGRAFYATETTRIIGDIRCAHCNISRT |
| 162.7 | 17 | SVEINCTRPGNNTRGSIHLHPGRKFYYSRGIIGDIREAHCAINIP |
| 170.5 | 7 | SVEINCTRPNNNTRRSVRIGPGGAMFRTGDIIGDIRCAHCNLSRT |
| 34.29 | 39 | SIEINCTRPNNNTRKSIQIGPGRAFYTTGEIIGDIRCAHCNLSRA |
| 74.17 | 94 | SVEINCTRPNNNTRKRMTLGPGKVFYTTGEIIGDIRKAHCNISRA |
| 396.2 | 2 | SVAINCTRRNNNTRRNIHIGLGRRFYATEIIGDTKKACCNISRA |
| 23.13 | 25 | SVEINCTRPIKIERKRIPLGLGKAFYTTKQVGDIKCAHC |
| 32.15 | 36 | PVEINCTRPNNNTRRSIHIAPGRAFYTTGQIIGDIRRAHCNISRT |
| 57.2 | 21 | TVVINCTRPNRHTGKSIRMGLGRAWHTTREIIGDIRKAYCTLNGT |
| 36.29 | 46 | SVNINCTRPNNNTRKGIHIGPGRTFFATGDIIGDIRCAHCNLSRT |
| BAL V3 | | CTRPNNNTRKSIHIGPGRAFYTTGEIIGDIRICAHC |

All documents cited herein are incorporated in their entirety by reference.

1. A method of stabilizing a hairpin loop structure of a peptide that comprises residues of the V3 domain of gp120 and that includes a B cell neutralizing antibody epitope, said method comprising
adding or substituting on the C-terminal or N-terminal end of said hairpin loop one or more positively or negatively charged amino acids so that said stabilization is effected by hydrogen bonding between oppositely charged amino acids present at the C- and N-terminal ends of said hairpin loop.

2. The method according to claim 1 wherein at least one negatively charged amino acid is added on the C-terminal end of said loop.

3. The method according to claim 2 wherein from one to three negatively charged amino acids are added on the C-terminal end of said loop.

4. A peptide comprising a sequence selected from the group consisting of:

| | |
|---|---|
| X¹-SIHIGPGRAFY-X² | (SEQ ID NO: 164) |
| X¹-SINIGPGRAFY-X² | (SEQ ID NO: 165) |
| X¹-GIHMGPGKAIY-X² | (SEQ ID NO: 166) |
| X¹-MTLGPGKVFY-X² | (SEQ ID NO: 167) |
| X¹-GIHIGPGRTFF-X² | (SEQ ID NO: 168) |
| X¹-SIQIGPGRAFY-X² | (SEQ ID NO: 169) |

wherein X¹ comprises at least two contiguous negatively or positively charged amino acids and X² comprises at least two contiguous amino acids bearing a charge opposite to that of the amino acids of X¹.

5. The peptide according to claim 4 wherein said peptide comprises an amino acid sequence set forth in Table 5.

6. A composition comprising a multiplicity of immunogenic polypeptides, each of said polypeptides comprising the peptide according to claim 4.

7. The composition according to claim 6 wherein said composition further comprises a carrier.

8. The composition according to claim 6 wherein said composition further comprises an adjuvant.

9. An isolated nucleic acid sequence encoding the peptide according to claim 4.

10. A vector comprising the nucleic acid sequence according to claim 9.

11. A composition comprising at least one nucleic acid sequence according to claim 9 and a carrier.

12. A method of inducing an immune response in a mammal comprising administering to said mammal an amount of at least one peptide according to claim 4 in an amount sufficient to effect said induction.

13. A method of inducing an immune response in a mammal comprising administering to said mammal an amount of at least one nucleic acid sequence according to claim 9 in an amount sufficient to effect said induction.
